(19) United States
(12) Patent Application Publication
(10) Pub. No.: US 2015/0140555 A1
(43) Pub. Date: May 21, 2015

(54) METHOD OF IDENTIFYING FOETAL ERYTHROBLAST

(71) Applicant: National University of Singapore, Singapore (SG)

(72) Inventors: Mahesh Choolani, Singapore (SG); Sukumar Ponnusamy, Singapore (SG); Huoming Zhang, Singapore (SG); Qingsong Lin, Singapore (SG); Aniza Puteri Mahyuddin, Singapore (SG); Priya Kadam, Singapore (SG)

(21) Appl. No.: 14/403,538

(22) PCT Filed: May 23, 2013
(86) PCT No.: PCT/SG2013/000212

§ 371 Date: Nov. 24, 2014

(30) Foreign Application Priority Data
May 25, 2012 (SG) 201203910-3

(51) Int. Cl. G01N 33/569; C07K 16/28; C07K 14/705; C12N 5/078; C12Q 1/68

(52) U.S. Cl. CPC G01N 33/56966; C12N 5/0641; C12Q 1/6883; C07K 14/705; C07K 16/28; C12Q 2600/156; G01N 2800/387; G01N 2800/385
USPC 435/6.11; 435/7.25; 435/325; 530/350; 530/389.6

(57) ABSTRACT

There is provided a method for identifying at least one foetal erythroblast the method comprising: (a) detecting the expression of at least one foetal erythroblast specific marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DK-FZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289, wherein detection of the marker indicates the presence of the foetal erythroblast.

### FIELD OF THE INVENTION

The present invention generally relates to methods for identifying and/or isolating at least one foetal primitive nucleated red blood cell. In particular, the invention relates to a method of identifying at least one foetal primitive nucleated red blood cell in a sample by detecting at least one membrane protein specific to the foetal primitive nucleated red blood cell.

### BACKGROUND TO THE INVENTION

Currently, prenatal diagnosis of chromosomal and single gene disorders rely on foetal cells obtained by invasive procedures such as amniocentesis, chorionic villous sampling (CVS) or foetal blood sampling (FBS) for cytogenetic and/or molecular analysis. These invasive tests carry a small but significant risk of foetal miscarriage. On the one hand this limits the uptake of the diagnostic test out of fear of foetal loss, and on the other hand causes the demise of an otherwise healthy foetus.

Non-invasive methods to diagnose the foetal genetic condition by enriching and analyzing foetal cells and foetal DNA that circulate in maternal blood have been studied.

Of the foetal cells that enter the first trimester maternal circulation, primitive foetal nucleated red blood cell (FPNRBC) is the preferred target cell. This is because of its short life-span and hence it is unlikely to persist from a previous pregnancy, unlike the situation with foetal lymphocyte where this phenomenon could be the basis for a misdiagnosis. First-trimester FPNRBC contain Epsilon-globin [symbol] , an ideal foetal cell identifier which is highly specific as expression declines after the first trimester.

In humans, foetal primitive nucleated red blood cells (FPNRBCs, foetal primitive erythroblasts, first trimester foetal nucleated blood cells (FNRBCs)) generated in the yolk sac mesoderm remain the predominant blood cell type in the embryonic circulation until 10 weeks post-conception. Studies on this cell type in humans have been limited owing to limited access to pure populations of these cells for laboratory investigations; only recently has it been shown that these cells may enucleate within the first trimester human placenta, suggesting that may be terminally differentiated. Primitive erythroblasts differ from foetal definitive erythroblasts not only in their anatomical site of origin, but also in the types of haemoglobins contained within them.

Adult anucleate red blood cells (AARBCs, adult red blood cells (RBCs)) are smaller, discoid, readily deformable cells that are produced in the long bone marrow. Owing to their ready availability, these cells have been extensively studied in recent years. Using mass spectrometry, AARBC membrane and cytoplasmic proteins have been characterized, and differences demonstrated between mouse and human AARBCs.

Enrichment of first trimester FPNRBC from maternal blood for non-invasive prenatal diagnosis has been a difficult task due to the lack of unique antibodies against its surface proteins. While WBCs can be separated using anti-CD45 antibody from maternal blood samples, separation of FPNRBCs from overwhelming adult RBCs has been the challenge. The success of non-invasive prenatal diagnosis using first trimester FPNRBCs from maternal blood depends on the enrichment of these rare cells (one cell amongst a million nucleated maternal cells).

The goal of isolating and analyzing foetal DNA from as little as one FPNRBC recovered from amongst a million nucleated maternal cells is possible with the use of automated micromanipulation, laser capture microscopy systems and downstream analysis of foetal cell with single cell whole genomic amplification coupled with array CGH technologies. Therefore, it is not inconceivable that very small numbers of foetal cells (˜20 cells) enriched from maternal blood from an on-going euploid pregnancy may actually be sufficient for non-invasive prenatal diagnosis.

Accordingly, there is a need in the art for a method for detecting and/or isolating FNRBCs and provide methods as potential reliable approaches for future NIPD using FNRBCs present in maternal blood.

### SUMMARY OF THE INVENTION

According to one aspect of the invention, there is provided a method for identifying and/or isolating at least one foetal erythroblast, the method comprising: detecting the expression of at least one foetal erythroblast specific marker selected from the group consisting of neutral amino acid transporter B (SLC1A5), solute carrier family 3 (activators of dibasic and neutral amino acid transport) member 2 isoform A (SLC3A2), splice isoform A of chloride channel protein 6, transferrin receptor protein 1, splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, antibacterial protein FALL-39 precursor, CAAX prenyl protease 1 homolog, splice isoform 2 of synaptophysin-like protein, vitamin K epoxide reductase complex subunit 1-like protein 1, splice isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, hypothetical protein MGC14288 (IPI Accession No. IPI00176708), protein with IPI Accession No. IPI00639803, and protein with IPI Accession No. IPI00646289; wherein detection of the marker indicates the presence of the foetal erythroblast.

According to other aspects of the invention, there is also provided a marker or identifying foetal erythroblast selected from the foetal erythroblast specific marker according to any aspect of the present invention, a method of diagnosing at least one prenatal disorder in an individual using at least one foetal erythroblast specific marker, an antibody or antigen binding fragment thereof that is capable of binding to at least one foetal erythroblast specific marker, and a kit for identifying and/or isolating foetal erythroblast in a sample.

### BRIEF DESCRIPTION OF THE FIGURES

FIG. 1 is histological images of FPNRBCs and AARBCs stained with Wright's stain showing (A) FPNRBCs (nucleated); (B) AARBCs without nuclei.

FIG. 2 is a Venn diagram of FPNRBC proteins identified in organic solvents MeOH and TFE.

FIGS. 3A-B are graphs showing the locations and function of 133 FPNRBC membrane proteins.

FIG. 4 is a Venn diagram of membrane proteins with potential surface domains in AARBCs and FPNRBCs.

FIG. 5 is images of validation of unique membrane proteins of FPNRBCs by reverse transcriptase-PCR (RT-PCR).

FIG. 6A is images of immunohistochemistry of membrane proteins on FPNRBCs and AARBCs.

FIG. 6B is box plot showing the statistical significance (*) of intensities of immunoreaction by antibodies.

FIG. 6C is a bar graph showing the statistical significance (*) of staining intensity of immunoreaction by antibodies.

FIG. 6D is images of immunohistochemistry of membrane proteins on FPNRBCs and AARBCs.

FIG. 7A is images of immunohistochemistry of membrane proteins on FPNRBCs and AARBCs in pre-sort and post-sort fraction with NAT-B marker.

FIGS. 7B and 7C are bar graphs showing the percentage FPNRBCs in NAT-B positive fraction, NAT-B negative fraction pre-sort and post-sort.

FIG. 8 is a table presenting data on proteins identified based on single peptides from TFE and MeOH extractions and ion score.

FIG. 9 is a table presenting peptide sequences for proteins identified from TFE and MeOH extractions.

### DETAILED DESCRIPTION OF THE PREFERRED EMBODIMENTS

Bibliographic references mentioned in the present specification are for convenience listed in the form of a list of references and added at the end of the examples. The whole content of such bibliographic references is herein incorporated by reference.

Reference to an element by the indefinite article “a” or “an” does not exclude the possibility that more than one of the element is present, unless the context clearly requires that there be one and only one of the elements. The indefinite article “a” or “an” as used herein thus usually means “at least one”.

The term “comprising” is herein defined as “including principally, but not necessarily solely”. Furthermore, the term “comprising” will be automatically read by the person skilled in the art as including “consisting of”. The variations of the word “comprising”, such as “comprise” and “comprises”, have correspondingly varied meanings.

The term “fragment” is herein defined as an incomplete or isolated portion of the full sequence of a protein which comprises the active/binding site(s) that confers the sequence with the characteristics and function of the protein. In particular, it may be shorter by at least one amino acid. More in particular, the fragment comprises the binding site(s) that enable the protein to bind to at least one marker of the present invention.

The term “antigen binding fragment” is herein defined as an incomplete or isolated portion of the full sequence of an antibody which comprises the active/binding site(s) that confers the sequence with the characteristics and function of the antibody. In particular, it may be shorter by at least one amino acid. More in particular, the fragment comprises the binding site(s) that enable the antibody to bind to at least one marker of the present invention.

The term “erythroblast” as used herein refers to a red blood cell having a nucleus. In particular, an erythroblast refers to a nucleated precursor cell from which a reticulocyte develops into an erythrocyte. “Erythroblast” may be used interchangeably with a “Normoblast” and refers to a nucleated red blood cell, the immediate precursor of an erythrocyte. For example, the erythroblast may be of mammalian origin. In particular, the erythroblast may be a primitive or human foetal erythroblast. The term “foetal primitive nucleated red blood cell (FPNRBC)” is herein defined as cells generated in the yolk sac mesoderm that remain as the predominant blood cell type in the embryonic circulation until 10 weeks post-conception. The term “FPNRBC” may be used interchangeably with foetal primitive erythroblasts or first trimester foetal nucleated red blood cells (FNRBCs)).

The phrase “adult anucleate red blood cells (AARBCs, adult red blood cells (RBCs))” is herein defined as cells that are relatively smaller as compared to FPNRBC, discoid, readily deformable and produced in the long bone marrow. The term AARBCs may be used interchangeably with “adult red blood cells (RBCs)”.

The term “mammalian” is herein defined as a mammalian individual, in particular, a primate for example a human being. For purposes of research, the subject may be a non-human. For example the subject may be an animal suitable for use in an animal model, e.g., a pig, horse, mouse, rat, cow, dog, cat, cattle, non-human primate (e.g. chimpanzee) and the like.

The term “sample” as used herein refers to a subset of tissues, cells or component parts (for example fluids) that may include, but are not limited to, maternal tissue, maternal blood, cord blood, amniocenteses, chorionic villus sample, foetal blood, and/or foetal tissue/fluids. In particular, foetal tissue may be trophoblast tissue, placental tissue or a combination thereof. The sample as used in the present invention may have been previously subjected to a density gradient purification including, but not limited to, Ficoll gradient and Percoll gradient.

The term “CD45 negative” as used herein refers to any cell that expresses no signal or is negative for native, recombinant or synthetic forms of the CD45 molecule/marker. The presence of CD45 expression on a cell in a sample may be determined using any immunostaining method known in the art and using any anti-CD45 reagent. Any cells positively stained with anti-CD45 reagent may be excluded as these may include CD45 positive white blood cells (WBC).

The term “nucleated” as used herein refers to a cell that has a nucleus. Nucleated cells may be distinguished from red blood cells which are not nucleated based on any nuclear staining known in the art.

The term “prenatal disorder” as used herein refers to diseases or conditions in a foetus or embryo before it is born. The prenatal disorder may be selected from the group consisting of a chromosomal disorder, a genetic disorder, or a combination thereof. In particular, the prenatal disorder may be selected from the non-limiting group consisting of Down Syndrome, Edwards Syndrome, Patau Syndrome, a neural tube defect, spina bifida, cleft palate, Tay Sachs Disease, sickle-cell anemia, thalassemia, cystic fibrosis, fragile X syndrome, spinal muscular atrophy, myotonic dystrophy, Huntington's Disease, Charcot-Marie-Tooth disease, haemophilia, Duchenne muscular dystrophy, mitochondrial disorder, Hereditary multiple exostoses, osteogenesis imperfecta disorder, a combination thereof and the like.

At present, enrichment of FPNRBCs from maternal blood has been a challenge because of their rarity in maternal circulation and the lack of surface specific antigens for immunocell sorting of these cells. CD71 and GPA are commonly used to enrich these cells from maternal blood: as such use of CD71 may result in loss as this surface antigen is expressed only on ˜68% of FPNRBCs and GPA binds to both. AARBCs and FNRBCs making analyses of enriched sample difficult because of a very high background of AARBCs.

Cell surface membrane proteins have an integral role in maintaining health: when altered structurally or functionally, they are responsible for the more commonly known diseased states such as spherocytosis and sickle cell disease, and also the less commonly recognized conditions such as elliptocytosis, familial pseudohyperkalaemia, dehydrated hereditary stomatocytosis and membrane defects in β-thalassemia. Knowledge about cell membrane proteins and their functions in health and disease could lead to understanding mechanisms of disease processes such as the invasion of the malaria parasite into human erythrocytes and the possibility of developing therapeutic interventions.

In contrast to the large amount of information already available on the AARBC membrane proteome, no information is currently available on the proteome of human foetal primitive erythroblasts. Only very limited data on their cell surface antigens such as CD71 and Glycophorin A and some information on their cytoplasmic haemoglobin are known. The knowledge on the membrane proteome of the FPNRBC may be useful in two ways: to facilitate a deeper understanding of primitive erythropoiesis in humans, and to identify specific surface antigen(s) for the enrichment of ε-globin-positive foetal primitive erythroblasts from maternal blood for non-invasive prenatal diagnosis. It has been suggested that the ε-globin-positive foetal primitive erythroblast is the ideal foetal cell type for non-invasive prenatal diagnosis and identification of unique membrane proteins on either FPNRBC or AARBC may be exploited for non-invasive prenatal diagnosis in the future. Differences between human FPNRBCs and AARBCs are disclosed herein. Accordingly, there is a need in the art to provide markers that facilitate the identification and/or isolation of FPNRBCs.

The inventors of the present application made the first attempt to explore unique membrane proteins of FPNRBCs. They identified unique surface proteins with transmembrane domains that may be useful as markers for the separation of human FPNRBCs from adult RBCs by immuno-cell sorting protocols. Antibodies against these proteins may enable the immuno-cell sorting.

According to an aspect of the present invention, there is provided a method for identifying at least one foetal erythroblast the method comprising: detecting the expression of at least one foetal erythroblast specific marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289; wherein detection of the marker indicates the presence of the foetal erythroblast. In particular, the foetal erythroblast is of mammalian origin. More in particular, the foetal erythroblast is of human origin. A brief description of individual foetal erythroblast specific markers, on their location, physiological roles (including those related to human foetal development), and diseases related to their mutations is provided below.

The identification of foetal erythroblast specific marker will facilitate the identification and isolation of FNRBCs from maternal blood, and thus provides for a reliable approach for future NIPD using FNRBCs present in maternal blood.

Brief Description of the Foetal Erythroblast Specific Markers

Amino Acid Transporters

Transporters of cells and organelles regulate the uptake and efflux of important compounds such as sugars, amino acids, nucleotides, ions and drugs. Solute carrier (SLC) series of transporters include genes encoding passive transporters, ion transporters and exchangers.

Two amino acid transporting SLC proteins (SLC1A5 and SLC3A2) were identified unique to plasma membrane of foetal primitive erythroblasts. SLC1A5, neutral amino acid transporter B⁰, is a Na⁺ dependent transporter of SLC1 family expressed in kidney and intestine. SLC1A5 amino acid transport protein identified in foetal erythroblasts belongs to ASCT2 system which can transport glutamine and asparagine with high affinity, and neutral amino acids methionine, leucine and glycine with low affinity.

SLC3A2 (CD98hc) is the heavy chain of the hetero-dimeric protein 4F2 (CD98). CD98 as a hetero-dimer, is involved in amino acid transportation where the substrate specificity varies with the nature of the light chain. Different domains of CD98hc are necessary for association with light chains. Studies on the amino acid transport in human placenta is correlated well with the expression of mRNAs of CD98hc, and a possible role for these proteins in materno-foetal transfer of amino acids and iodo-thyronines is also suggested. CD98hc is found to be co-localized with α₄β₃ integrin to promote adhesion and motility of extravillous trophoblasts suggesting the functional importance of CD98hc in human foetal development.

There is evidence for amino acid transport in matured human red blood cells too; It has been previously demonstrated that a Na+ dependent amino acid transport system, and recently, CD98hc associated with L-type amino acid transporter 1 (LAT1) or LAT2 light chain may be involved in the cellular uptake of S-nitroso-L-cysteine into human adult red blood cells. However, the absence of CD98hc in mass spectrometric studies of AARBCs may probably be due to smaller peptides generated during MS.

Anion Transporters

Chloride channel (Cc) genes (Clc1-10) are expressed in all phyla from bacteria to man. Clc mediated anion transport is considered to be the main function of most of the Clc proteins.

Three isoforms of Clc6 are known. Mutations in Clc genes have been implicated in various human diseases such as myotonia, renal salt loss, deafness, urinary protein loss, kidney stones, osteoporosis, blindness, and lysosomal storage disease. Recent studies in animal models suggest that Ccl6 may predominantly reside intracellularly in endosomes. In AARBCs, in addition to a small chloride channel other inorganic ion transporters, such as urea transporter-B (SLC14A1) and bicarbonate/chloride exchanger (SLC4A1, Band 3), are known to be functional.

Binding Proteins

Membrane receptors which can bind hormones, growth factors and metabolites are important for cellular growth and function. Transferrin receptor protein 1, Splice isoform 2 of protein GPR107 precursor, and olfactory receptor 11H4 were identified as being unique to primitive foetal erythroblasts. Transferrin receptor was initially identified on maturing erythroid cells and placenta. Iron is an essential requirement for the synthesis of haemoglobin in all stages in erythroid cells to where iron is transported by the transferrin receptor which, however, is absent in AARBCs as it is lost from reticulocytes as they become mature.

Guanine nucleotide binding protein (G protein) coupled receptors (GPCRs) have 7 transmembrane helices and are expressed on cell surface, and bind to almost all of the known neurotransmitters and hormones released synaptically or those that are secreted into the circulatory system controlling organ functions. G-proteins are predominant intracellular molecules that bind and link GPCRs to second messenger systems such as adenyl cyclase, phospholipases, and ionic conductance channels. GPCRs are targets for 40% of all approved drugs and are the main focus of intense pharmaceutical research due to their key roles in cell physiology and disease, and the presence of GPR107 in foetal erythroblasts does not exclude the possibility for potential research using this cell type for foetal therapy.

Olfactory receptor (OR) is the largest mammalian gene family that codes for odorant receptors. Identification of one of the ORs (OR family H subfamily 11) in primitive foetal erythroblasts supports the earlier reports of an OR in hematopoietic cells and tissues: low level expression of OR-mRNA in human erythroleukemia and myeloid cell lines, and in tissues containing cells of erythroid lineage, such as human bone marrow and foetal liver were reported by Feingold and his colleagues. There is evidence for the expression of OR in non-olfactory testicular tissue; in humans, expression of hOR 17-4 and its functional role in sperm chemotaxis is known. In addition, human prostate specific G-protein coupled receptor (PSGR) with properties characteristic of an olfactory receptor was also observed in olfactory zone and the medulla oblongata (human), liver (rat) and in brain and colon (mouse).

Catalytic

CAAX prenyl endopeptidase also known as FACE, farnesylated protein-converting enzyme, is important for prenylation of CAXX motif containing eukaryotic proteins for their function and membrane targeting. FACE-1 and FACE-2 are two human enzymes expressed in several tissues, for example, leukocytes, ovary, testis, kidney and placenta. Prelamin-A is the substrate for FACE-1 and mutations in prelamin A cleavage site or FACE-1 enzyme have been documented in genetic diseases such as Hutchinson-Gilford progeria and mandibuloacral dysplasia. The identification of CAAX prenyl protease 1 homologue, an integral membrane protein containing seven transmembrane domains in foetal erythroblasts, as in other human tissues, indicates a possible house-keeping role for this enzyme in the processing of prenylated proteins.

Vitamin K epoxide reductase complex subunit 1 like protein (VKORC1L1), identified in the present disclosure, is the first report in a human erythroid cell type membrane protein whose sub-cellular location is not yet defined. VKORC1 was reported to be warfarin-sensitive. Vitamin K-dependent clotting factor deficiency type 2 (VKCFD2) in humans showing warfarin resistance is the result of mutation in VKORC1. Foetal warfarin syndrome (warfarin embryopathy) due to warfarin exposure during pregnancy is well known. It has also been suggested that rare polymorphisms and interethnic differences in VKORC1 determines warfarin requirement.

Signaling Pathway

Splice isoform 1 of Protein C9ORF5 identified in primitive foetal erythroblasts is annotated to be involved in signalling pathways. A novel human transcript CG-2 (C9ORF5) was isolated from the familial dysautonomia candidate region on 9831 and its expression was seen in human adult and foetal tissues such as brain, lung, liver and kidney. C9ORF5 was also found to be upregulated in prostrate cancer where the role for this gene is unknown.

Vesicle Recycling

Synaptophysin-like protein, pantophysin, an isoform of synaptophysin identified in primitive erythroblasts was annotated to be located in plasma/vesicle membrane. It is highly conserved and considered as a novel pre-synaptic marker for neurons and neuroendocrine (NE) cells. Pantophysin is localized in cytoplasmic micro-vesicles of various secretory, shuttling, and endocytotic recycling pathways and are co-localized with synaptophysin in transfected non-neuroendocrine and neuroendocrine cells and in neuroendocrine tissues. Non-neuronal distribution of pantophysin in epithelial, muscle tissues and fibroblasts has already been documented.

Antimicrobial Proteins

Expression of BCG induced integral membrane protein BIGM 103 (BCG induced gene in monocyte, clone 103) in foetal erythroblasts is novel. This protein was first identified from cDNA library prepared from monocytes induced with BCG cell wall. BIGM103 has sequence similarity with Zip-like family of proteins and matched with hZIP2 and hZIP1 and is predicted to possess zinc transporter and metallo-protease activities. A possible role in phagocytosis-mediated elimination of microbial components in macrophages and dendritic cells has also been suggested. FALL39 identified in foetal erythroblasts is one of the antimicrobial peptides of neutrophil granules such as Azurocidin (CAP-37) and CAP-57. FALL39 was also identified from human bone marrow and testis. Contrary to the microbicidal function, a novel pro-tumorigenic role for mature FALL-39 (hCAP-18/LL-37) was also demonstrated in ovarian cancer, through activation of matrix metalloproteinases, and there is evidence for strong association between leukocyte infiltration and cancer progression.

Proteins with No Known Function but Candidates for Research Related to Foetal Development.

Cleft lip and palate transmembrane protein 1—To date, no functional role for CLPTM 1 is defined. CLPTM 1 is reported to be homologous with Cisplatin Resistance Related gene-9, and observed to be more expressed in clinical samples resistant to chemotherapy in breast cancer. Clinically, folate deficiency is known to be associated with cleft lip and/or palate and auto-antibodies against folate receptors are reported to be present in mothers of children with cleft lips. Folate is an important vitamin for several metabolic pathways including those leading to the synthesis of nucleic acids, and are considered vital during infancy and pregnancy. Functional role for CLPTM 1 in foetal erythroblast plasma membrane needs further investigation.

Hypoxia-inducible gene 1 protein, (HIG1 domain family member 1A, HIGD1A) is one of the genes expressed during hypoxia. HIGD1A gene expression was reported in human hematopoietic stem/progenitor cells and in human cervical cells cultured under hypoxic conditions. HIG1 expression in cytoplasmic vesicles and mitochondria appears to be induced by both hypoxia and tumour micro environmental stressor such as glucose deprivation. In humans, the normal foetal development depends on the availability of oxygen and nutrients to the foetus. Identification of HIGD1A protein expression in primitive foetal erythroblasts, but not in adult erythrocytes, correlates with the relatively hypoxic environment of the placenta as compared to that of adult blood circulation.

Others

Identification of ALEX3 protein variant in foetal erythroblasts is unique. The genes for ALEX1, ALEX2 and ALEX3 are localized in human X chromosome. Significantly reduced or loss of mRNA expression of ALEX1 and ALEX2 in epithelial carcinomas (human lung, prostate, colon, pancreas, and ovarian carcinomas) but not in cell lines from other types of tumours leads to a speculation that ALEX genes may play a role in suppression of tumours originating from epithelial tissue.

Reports on protein expression or functional identity of five of the identified proteins of foetal erythroblasts (with at least one transmembrane domain) are not available in any other cell/tissue; they are, Hypothetical protein DKFZp586C1924, 8 kDa protein, 25 Kda protein, Hypothetical protein MGC14288, and Splice Isoform 1 of Protein C20orf22 (ABHD12). Protein databases searches (UniProtKB/Swiss-Prot) did not reveal much information for these proteins. Recently, mRNA expression of Hypothetical protein DKFZp586C1924(TMEM 126A) in human foetal and adult tissues and immuno-localization in mouse mitochondria have been reported.

According to another aspect of the invention, there is provided a method for identifying at least one foetal erythroblast comprising detecting the expression of at least one foetal erythroblast specific marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, antibacterial protein FALL-39 precursor, CAAX prenyl protease 1 homolog, splice isoform 2 of synaptophysin-like protein, and splice isoform 1 of Protein C20orf22 (ABHD12), wherein detection of the marker indicates the presence of the foetal erythroblast. In particular, the detecting comprises detecting the expression of at least one foetal erythroblast specific marker selected from the group consisting of splice isoform 1 of Protein C20orf22 (ABHD12), Splice Isoform 3 of Protein GPR107 precursor, Olfactory receptor 11H4, and ALEX3 protein variant.

Alternatively, the foetal erythroblast specific marker may be detected by an antibody, antigen binding fragment thereof, or the like. In particular, the antibody may be polyclonal or monoclonal. A person skilled in the art would understand that any molecular or compound capable of recognizing and/or binding to the foetal erythroblast specific marker can be used to detect the foetal erythroblast specific marker.

According to another aspect of the invention, there is provided a method of isolating at least one foetal erythroblast from a sample, the method comprising: (a) contacting the sample with at least one antibody or antigen binding fragment thereof that is capable of binding to at least one marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289; and (b) isolating the foetal erythroblast that binds to the antibody or antigen binding fragment thereof from the sample.

In particular, the antibody may be a polyclonal antibody, a monoclonal antibody, a chimeric antibody, a humanized antibody or a combination thereof. More in particular, the foetal erythroblast that binds to the antibody is isolated from the sample using immunomagnetic separation, flow cytometry or a combination thereof.

The isolation of the mammalian nucleated foetal cell from the sample may be performed using, but not limited to, a micromanipulator or any system that allows individual picking of a foetal cell. In particular, the foetal cell may be a mammalian foetal erythroblast. More in particular, the foetal cell may be a primitive or human foetal erythroblast.

Density gradients and flow sorting methods may be employed to enhance enrichment and purity of foetal erythroblasts from maternal blood.

According to yet another aspect of the invention, there is provided a method of diagnosing at least one prenatal disorder in an individual, the method comprising: a. identifying at least one foetal erythroblast in a sample of the individual according to the method described above; b. isolating the foetal erythroblast; and c. determining at least one genetic marker associated with the prenatal disorder in the foetal erythroblast. In particular, the prenatal disorder may be selected from the group consisting of Down Syndrome, Edwards Syndrome, Patau Syndrome, a neural tube defect, spina bifida, cleft palate, Tay Sachs disease, sickle-cell anemia, thalassemia, cystic fibrosis, fragile X syndrome, spinal muscular atrophy, myotonic dystrophy, Huntington's disease, Charcot-Marie-Tooth disease, haemophilia, Duchenne Muscular Dystrophy, mitochondrial disorder, hereditary multiple exostoses and osteogenesis imperfecta disorder. More in particular, the sample may be selected from the group consisting of maternal tissue, maternal blood, cord blood, amniocytes, chorionic villus sample, foetal blood, and foetal tissue. In particular, the method may be carried out in vitro.

According to an aspect of the invention, there is provided a marker for identifying foetal erythroblast selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289. There is further provided an antibody or antigen binding fragment thereof that is capable of binding at least one marker according to the present invention.

Also provided is a kit for use in a method of identifying and/or isolating foetal erythroblast according to any aspects of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Material and Methods

An in-depth literature search was conducted on the presence and functional roles of unique plasma membrane proteins of FPNRBCs in various human tissues and cells, including that of foetus (trophoblasts/placenta). Short description of these proteins, on their location, physiological roles (including those related to human foetal development), and diseases related to their mutations have been provided above, together with available data on similar functions in AARBCs.

FPRNBCs can be separated from WBCs in maternal blood by negative depletion of CD45 positive cells, and if suitable surface antigen known on FPNRBCs available, these ideal cells for non-invasive prenatal diagnosis can be enriched from AARBCs. Membrane proteins of FPNRBCs were profiled by mass spectrometry, and compared this profile with that of the AARBC membrane proteome as known in the art to identify unique surface membrane proteins of FPNRBCs which are absent in AARBCs.

Membrane proteins of FPNRBCs profiled by mass spectrometry may be compared to known membrane proteome of AARBC. A shot-gun proteomics approach, two-dimensional liquid chromatography coupled with MALDI-TOF/TOF-MS (2D-LCMS/MS) was used to characterize the membrane proteome of foetal primitive erythroblasts. This is the first report on the membrane proteome of the foetal primitive erythroblasts. Details of all 273 proteins identified are provided including their annotated sub-cellular locations, molecular functions and number of transmembrane domains. 133 (48.7%) proteins were membrane proteins, of which 37 were plasma membrane proteins.

Unique, surface membrane proteins of FPRNBCs were identified by comparing the data of the present study with membrane proteins of AARBCs to identify common, and 12 plasma membrane proteins with transmembrane domains and 8 proteins with transmembrane domains but without known sub-cellular location were identified as unique-to-FPNRBCs. Except for the transferrin receptor, all other 19 unique-to-FPNRBC membrane proteins have never been described in red blood cells. Reverse-transcriptase PCR (RT-PCR) and immunocytochemistry validated the 2D-LCMS/MS data. The findings provide potential surface antigens for separation of FPNRBCs from maternal blood for non-invasive prenatal diagnosis, and help understand the biology these rare cells.

Proteomic analyses of FPNRBCs had not been attempted previously owing to the difficulty to obtain sufficient number of cells. Access to placental villi from patients undergoing termination of pregnancy enabled to pool cells for 2D-LCMS/MS analysis. In addition, the extraction of membrane proteins is yet another challenge in proteomics; recovery of more membrane proteins (48.7% of total) from a limited sample (5×10⁷ cells) than those from AARBCs using similar protocol is encouraging, which also explains the structural complexity of these nucleated cells.

Sub-cellular localization and molecular functions annotated for most of the proteins of FPNRBCs are novel for this cell type. Identified FPNRBC membrane proteins show diverse physiological functions varying from transport, catalytic, binding to structural, while about 32% were transport and/or catalytic. Among the membrane proteins, most were identified from mitochondria (48 proteins) and plasma membrane (37 proteins).

Tissues

Placental tissue collection from women undergoing elective first trimester surgical termination of pregnancy was approved by the Institutional Review Board, and all patients gave written informed consent.

Extraction of FPNRBCs from Placental Villi

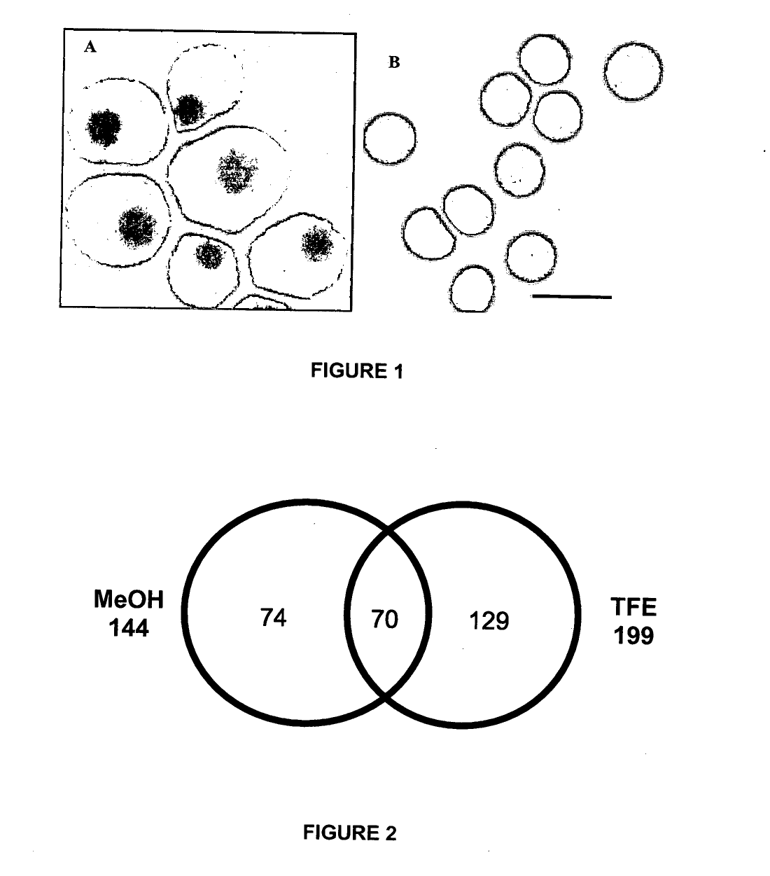

FPNRBCs were extracted from placental villi, and AARBCs were prepared from volunteer blood sample. Placental tissues were collected at the termination of pregnancy (7⁺⁰ to 9⁺³ weeks amenorrhoea). FPNRBCs were extracted from placental villi as per protocol known in the art. Placental villi were digested in trophoblast digestion buffer (146.3 ml HBSS containing 0.182 g trypsin and 3.75 ml 1M Hepes (Gibco®-Invitrogen-Life-Technologies, NY, USA) for 30 min at 37° C. in a shaking-water-bath, and digestion was stopped using foetal calf serum (Pierce, Ill., USA) (5 ml/45 ml digestion buffer). Single cell suspensions were centrifuged (3000 rpm, 20° C., 10 min). Red cell pellets containing FPNRBCs were suspended in PBS, and separated using Percoll 1083 (GE Healthcare, Uppsala, Sweden) (3000 rpm, 20° C., 20 min). FPNRBC purity was determined by basic staining of cytospun slides. Samples were stored for membrane preparation (if purity≧90% FPNRBCs) in HES buffer (20 mM HEPES, pH 7.4, 1 mM EDTA and 250 mM sucrose) with protease-inhibitor cocktail (Roche Diagnostics, Mannheim, Germany) at −80° C. Morphologies of FPNRBCs and AARBCs are shown, in FIG. 1. Bright field images were captured using 20×/0.40 PhP objective lens of CKX41 Olympus microscope. Bar represents 10 μm.

Membrane Protein Preparation and Digestion

Membranes from pooled FPNRBCs (5×10⁷ cells) were prepared as described in the art. Cells stored in HES buffer were lysed by thawing and sonication, and ultra-centrifuged at 100,000×g 4° C. (1 h) to obtain the membrane pellet which was then washed using high pH solution (0.1M Na₂CO₃, pH11), and twice with Milli-Q water. Proteins were extracted from FPNRBC membranes using methanol (MeOH)/50 mM NH₄HCO₃ (60:40, vol/vol), and protein reduction, alkylation and digestion were carried out as described by Blonder et al. Tryptic digestion was carried out using sequencing grade modified trypsin (Promega, Southampton, UK). Digested sample was centrifuged and the pellet washed in MeOH solution (60% MeOH in 50 mM NH₄HCO₃) twice. Supernatants were pooled (MeOH-derived digests), while the pellet was re-suspended in Trifluoroethanol (TFE)/50 mM NH₄HCO₃ (50:50 vol/vol) and the proteins extracted were then diluted 10 times with 50 mM NH₄HCO₃ for a second trypsin digestion to obtain supernatants (TFE-derived digests). Both digests were lyophilized and stored at −80° C.

Two-Dimensional Liquid Chromatography and Mass Spectrometry (2D-LCMS/MS)

2D-LCMS/MS was essentially the same described earlier by us (Zhang et al., 2007). Lyophilized digests were re-suspended in solvent [(98% H₂O, 2% acetonitrile (CAN) and 0.05% trifluoroacetic acid (TFA)], and after centrifugation supernatants were separated using an Ultimate-Dual-HPLC system (Dionex, Sunnyvale, Calif., USA). All samples were first separated on a strong cation exchange (SCX) column (300 μm i.d., ×15 cm, packed with 10 μm POROS 10S) and eluted fractions were captured on the PepMap trap column (300 μm i.d., ×1 mm, packed with 5 μm C18 100 Å), and eluted by gradient elution to a reversed-phase column (Monolithic Capillary Column, 200 μm i.d., ×5 cm). LC fractions were mixed with matrix-assisted laser desorption/onization (MALDI) matrix (7 mg/ml α-cyano-4-hydroxycinnamic acid and 130 μg/ml ammonium citrate in 75% CAN) at a flow rate of 5.4 μl/min through a 25 nl mixing-tee (Upchurch Scientific, Oak Harbor, Wash., USA) before being spotted onto 192-well stainless steel MALDI target plates (AB SCIEX, Foster City, Calif., USA), at a rate of one well per 5 s, using a Probot Micro Fraction collector (Dionex).

Samples on the MALDI target plates were analyzed using an ABI 4700 Proteomics Analyzer (AB SCIEX) with a MALDI source and time of flight analyzer TOF/TOF™ optics. For MS analysis, typically 1000 shots were accumulated for each sample well. Tandem-MS_(MS/MS) analyses were performed using nitrogen, at collision energy of 1 kV and a collision gas pressure of ˜3.0×10⁻⁷ Torr. 3000 to 6000 shots were combined for each spectrum depending on the quality of the data.

Database Searching

MASCOT search engine (v2.0; Matrix Science) was used to search tandem mass spectra. GPS Explorer™ software (v3.6; AB SCIEX) was used to create and search files with the MASCOT search engine for peptide and protein identifications. The International Protein Index (IPI) human protein database (v3.10) was used for the search of tryptic peptides and 57478 entries were searched. All MS/MS spectra from the LC runs were combined for the search. Cysteine carbamidomethylation, N-terminal acetylation and pyroglutamination, and methionine oxidation were selected as variable modifications. Two missed cleavages were allowed. Precursor error tolerance was set to 200 ppm and MS/MS fragment error tolerance was 0.4 Da.

Estimation of False Positive Rate

The false positive rate was calculated by comparing the search results from a randomized database versus the actual database. The minimum ion score C.I. percent such that no more than 5% false discovery rate (FDR) was achieved and was used as the cut-off threshold at the peptide level. All the proteins identified from random database search were single peptide-matched. Proteins identified by this method from IPI human database were colour coded as red, green or black: those red coloured proteins are matched to at least two peptides and hence are statistically confident (FDR is zero); proteins that are green coloured are identified by single peptide where match scores are higher than the highest score in the decoy database and essentially the FDR is zero; black coloured proteins were identified based on single peptide match fall within the set threshold of 5% FDR. Top ranked peptides with Best Ion scores≧33 and 36 for TFE and MeOH extractions, respectively, were included for analysis as peptides counted for each protein. All the MS/MS spectra were further validated manually.

Annotation

Sub cellular and functional categories of the identified proteins were obtained based on annotations of Gene Ontology using GoFig. (http://udgenome.ags.udel.edu/gofigure/index.html). Swiss-prot and TrEMBL data base were also used for functional annotation of unique proteins of FPNRBCs. The number of transmembrane domains (TMD) of the identified proteins was predicted using TMHMM Server (v2.0) (http://www.cbs.dtu.dk/services/TMHMM/).

Evaluation of the Identified Unique Proteins

a) Reverse Transcriptase PCR (RT-PCR) for mRNA Expression of Unique Proteins

RNA extraction—RNA from FPNRBCs was isolated using an RNeasy Mini Kit (Qiagen, Germany) according to manufacturer's instructions. Briefly, FPNRBCs (3×10⁶ cells) were resuspended in 350 μl lysis buffer and passed through QIAshredder spin column. The lysate was mixed with 350 μl of 70% ethanol and pipetted onto an RNeasy mini column, and centrifuged at 15000×g for 15 sec. RNA trapped in the column was washed using 350 μl buffer RW1 and incubated with 10 μl of DNase in 70 μl RDD buffer at room temperature for 15 min. RNA was then washed twice with 350 μl of buffer RW1 and once with 500 μl buffer RPE and recovered by the addition of 50 μl RNase-free water onto the column and centrifugation at 15000×g for 1 min.

RT-PCR—cDNA template was synthesised using Sensiscript RT Kit (Qiagen, Germany). Briefly, 5 μl of RNA was mixed with oligo-dT, RNase inhibitor, dNTP mix and RNase-free water (as per manufacturer's instructions) and incubated at 70° C. for 5 min and chilled on ice. RT buffer and RT enzyme were added to the mixture and incubated at 25° C. (15 min), 42° C. (60 min) and 72° C. (15 min), and cooled on ice. PCR mixture contained 5 μl cDNA, 1×PCR buffer, 1 mM dNTP, 8 mM MgCl₂, 2.5 U Taq polymerase and 0.6 μM primers. Denatured (94° C. 2 min) mixture was amplified by 45 cycles of 94° C. for 15 sec, ˜60° C. (depends on primer pairs) for 15 sec, 72° C. for 1 min. A final extension at 72° C. for 4 min was performed for each gene. RT control (no enzyme in RT step) and PCR control (Water-blanks) were also included. PCR products were separated by electrophoresis in a 2% agarose gel, stained with ethidium bromide (0.5 g/ml) and visualized under UV light. The images were captured using a digital imager (Alpha Innotech Corp., San Leandro, Calif.). Primer pairs (Sigma-Proligo) used for the amplification for individual gene are listed in Table 1.

TABLE 1
Primer pairs used in mRNA expression studies by RT-PCR

| | | Forward | |
|---|---|---|---|
| Selected Proteins | | SEQ | |
| Unique Membrane | | ID | |
| Proteins of FPNRBCs | Gene name | NO: | |
| Neutral amino acid | SLC1A5 | 1 | 5′-TGGCTGCTGGAGTACATGTG-3′ |
| transporter B | | | |
| Sollute carrier family | SLC3A2 | 3 | 5′-ATGGACCCACTACCCTTCTC-3′ |
| 3 member 2, isoform A | | | |
| Splice isoform A of | CLCN6 | 5 | 5′-GGGACCTTGTGCTGAGGGA-3′ |
| Chloride channel | | | |
| protein 6 | | | |
| Transferrin receptor | TFRC | 7 | 5′-TAGGCAGCAGCTTTTAATACAGG-3′ |
| protein 1 | | | |
| Splice isoform 3 of | GPR107 | 9 | 5′-TCAGAACATGGTTGTTCTCCC-3′ |
| protein GPR107 | | | |
| precursor | | | |
| Olfactory receptor | OR11H4 | 11 | 5′-AACAACTGAATGTCTCTTTCT-3′ |
| 11H4 | | | |
| Splice isoform of | C9orf5 | 13 | 5′-TAGCCCTGACCTTGCAGTCT-3′ |
| protein C9orf5 | | | |
| Cleft lip and palate | CLPTM1 | 15 | 5′-AGGTTCCCACAGCAGCAG-3′ |
| transmembrane protein | | | |
| 1 | | | |
| BCG induced integral | SLC39A8 | 17 | 5′-GTCTGAGATGCCTGGTATATAG-3′ |
| membrane protein | | | |
| BIGM103 | | | |
| Antibacterial protein | CAMP | 19 | 5′-GATAACAAGAGATTTGCCCTGC-3′ |
| FALL-39 precursor | | | |
| CAAX prenyl protease | ZMPSTE24 | 21 | 5′-CCTAAGGCTAAAGAGGAGCAG-3′ |
| 1 homolog | | | |
| Synaptophysin-like | SVPL1 | 23 | 5′-TGCATCATAAAGGAACCTAAGTG-3′ |
| protein | | | |
| Vitamin K exposide | VKORC1L1 | 25 | 5′-AGACACCTCAGGCAGCACTT-3′ |
| reductase complex | | | |
| subunit 1-like protein | | | |
| Other Proteins | | | |
| Vesicle associated | VAMP2 | 27 | 5′-AGTCCCTTAACCTGCCACG-3′ |
| membrane protein | | | |
| Hemoglobin epsilon | HBE1 | 29 | 5′-TTTTACTGCTGAGGAGAAGGCTGCC-3′ |
| chain | | | |
| Hemoglobin gamma-2 | HBG2 | 31 | 5′-ACGCCATGGGTCATTTCACAGA-3′ |
| chain | | | |
| Band 3 anion transport | SLC4A1 | 33 | 5′-ACACAGCTCTTCGTGGAGCA-3′ |
| protein | | | |
| Glyeraldehyde-3-phos- | GAPDH | 35 | 5′-AAGGACTCATGACCACAGTCCATG-3′ |
| phate dehydrogenase | | | |
| Vacuolar proton trans- | ATP6V0A1 | 37 | 5′-ACCTGACCCGACCTTGTG-3′ |
| locating ATPase 116kDa | | | |
| subunit a isoform 1 | | | |
| CDNA PSEC0252 fis, | SLC34A3 | 39 | 5′-ATGTCCTAGAAGGTTTTAGG-3′ |
| clone NT2RP3003258, | | | |
| highly similar to | | | |
| Likely ortholog of | | | |
| mouse embryo | | | |
| Steroid dehydrogenase | HSD17B12 | 41 | 5′-TGAAATATGCAGCAAGAAGATTGG-3′ |
| homolog | | | |
| Azurocidin precursor | AZU1 | 43 | 5′-GTGCTGGGTGCCTATGACCTGAGG-3′ |
| Solute carrier family | SLC22A11 | 45 | 5′-CTGGGTTCCAATCTCACCC-3′ |
| 22 member 11, isoform | | | |
| 2 | | | |

| | Reverse | | |
|---|---|---|---|
| | SEQ | | Amplified |
| Selected Proteins | ID | | Size |
| Unique Membrane | NO: | | (base pairs) |
| Proteins of FPNRBCs | 2 | 5′-CCCAGTGGGGGCTAGAATTC-3′ | 196 |
| Neutral amino acid | | | |
| transporter B | | | |
| Sollute carrier family | 4 | 5′-CATGCAGGGGTGACTTTTAT-3′ | 150 |
| 3 member 2, isoform A | | | |
| Splice isoform A of | 6 | 5′-AGCTGCGACTGCGGCAAT-3′ | 246 |
| Chloride channel | | | |
| protein 6 | | | |
| Transferrin receptor | 8 | 5′-AAAGTAAGCGAACCACTTACAACC-3 | 238 |
| protein 1 | | | |
| Splice isoform 3 of | 10 | 5′-GCTTGCTCTTCCTCCACATC-3′ | 164 |
| protein GPR107 | | | |
| precursor | | | |
| Olfactory receptor | 12 | 5′-GGAGTCGTTACTGAATATACC-3 | 483 |
| 11H4 | | | |
| Splice isoform of | 14 | 5′-GCATTTGGAAGTAATGCTAGCC-3 | 123 |
| protein C9orf5 | | | |
| Cleft lip and palate | 16 | 5′-CCTCTGCTGGCTTTGGAG-3′ | 155 |
| transmembrane protein | | | |
| 1 | | | |
| BCG induced integral | 18 | 5′-TCTTTGGCTCCTTAAAGACTTGG-3′ | 314 |
| membrane protein | | | |
| BIGM103 | | | |
| Antibacterial protein | 20 | 5′-GGGTAGGGCACACACTAGGA-3′ | 146 |
| FALL-39 precursor | | | |
| CAAX prenyl protease | 22 | 5′-GCGTTGGCAATGTTTAATGT-3′ | 146 |
| 1 homolog | | | |
| Synaptophysin-like | 24 | 5′-TGTAAGAATAAGAAACCTGAATCCC-3′ | 144 |
| protein | | | |
| Vitamin K exposide | 26 | 5′-TATTTCACCTTTTCTGGGCG-3 | 134 |
| reductase complex | | | |
| subunit 1-like protein | | | |
| Other Proteins | | | |
| Vesicle associated | 28 | 5′-CTGGGATAATATGGGGGGTC-3′ | 165 |
| membrane protein | | | |
| Hemoglobin epsilon | 30 | 5′-CTTGCCAAAGTGAGTAGCCAGAATAA-3′ | 355 |
| chain | | | |
| Hemoglobin gamma-2 | 32 | 5′-GAGCTCAGTGGTATCTGGAGGA-3′ | 455 |
| chain | | | |
| Band 3 anion transport | 34 | 5′-TCCGACACTCCCATCTGGTT-3′ | 727 |
| protein | | | |
| Glyeraldehyde-3-phos- | 36 | 5′-TTGATGGTACATGACAAGGTGCGG-3′ | 673 |
| phate dehydrogenase | | | |
| Vacuolar proton trans- | 38 | 5′-CTGAACTCTGCTTCAAACCCC-3′ | 96 |
| locating ATPase 116kDa | | | |
| subunit a isoform 1 | | | |
| CDNA PSEC0252 fis, | 40 | 5′-CAAAGATAGTCTGTCAGAAA-3′ | 104 |
| clone NT2RP3003258, | | | |
| highly similar to | | | |
| Likely ortholog | | | |
| of mouse embryo | | | |
| Steroid dehydrogenase | 42 | 5′-AATGATGCTGATAGCAGATGGCT-3′ | 193 |
| homolog | | | |
| Azurocidin precursor | 44 | 5′-AAGAGCGCCACTCGGGTGAAGAA-3′ | 467 |
| Solute carrier family | 46 | 5′-TTTTTCTGGCAGCTCTCTCA-3′ | 150 |
| 22 member 11, isoform | | | |
| 2 | | | |

b) Localisation of Unique Proteins on FPNRBCs by Alkaline Phosphatase Immunocytochemistry

8 commercially available antibodies against unique proteins of FPNRBCs annotated to be on plasma membrane, and also in other membranes or unique proteins with unknown sub-cellular location were used to localize their antigens in both FPNRBCs and AARBCs: Neutral amino acid transporter B (SLC1A5) (Chemicon-International, Temecula, Calif., USA), Solute carrier family 3, member 2, isoform A (SLC3A2), Olfactory receptor 11H4 (OR11H4) and Antibacterial protein FALL-39 precursor (Cathelicidin antimicrobial peptide, CAP-18) (all from Abcam, Cambridge, UK), Cleft lip and palate transmembrane protein1 (CLPTM1), Armadillo Repeat-Containing X-linked protein 3 (ARMCX3/ALEX3), and CAAX prenyl proteasel homolog (FACE1) (all from Novus-Biologicals, Littleton, Colo.), and Chloride channel protein 6 (CLCN6) (Santa-Cruz Biotechnology, Inc., CA, USA). Cells were fixed for 10 min either with 4% paraformaldehyde for SLC1A5, SLC3A2, OR11H4, CLCN6, CLPTM1, ARMCX3 or ice-cold methanol:acetone (1:1) for CAP-18 and FACE1; Following steps were common for all slides: Briefly, nonspecific binding was inhibited with diluted goat serum (Sigma-Diagnostics, MO, USA) (1:10 in PBS) for 120 min which was followed by incubation with respective primary-antibodies (1:100) for 60 min at room temperature or overnight at 4° C. Slides were then incubated with corresponding mouse or rabbit biotinylated secondary-antibody (1:100) for 60 min (Vector-Laboratories, CA, USA). This was followed by incubation with streptavidin conjugated alkaline phosphatase (Vector-Laboratories) (1:100). Immunoreaction was detected with freshly prepared Vector-Blue-substrate (Vector-Laboratories) for 10 min in dark. All incubations were performed in a humidifying chamber at room temperature and washes between incubations were in 1×PBST (5 min). Slides were rinsed in water and nuclei stained with nuclear-fast-stain (10 min), slides were rinsed in water and dehydrated with 100% ethanol (30 secs each). Air dried slides were mounted with Vectashield (Vector-Laboratories) and analysed by light microscopy. The staining intensity for each antibody tested was calculated as described by Lehr et al. Mean pixel intensities calculated from the luminosity histogram function on Adobe Photoshop CS4 software (Adobe Systems, Mountain View, Calif.) were compared for statistical significance.

Isolation of FPNRBCs in Spiked Blood Samples

Spiked model mixtures (1×105 FNRBCs in 2 ml peripheral blood) were sorted by CD45 depletion (Magnetic associated cell sorting) and NAT-B positive selection. The enriched mixture was tested for FPNRBCs recovery by haemocytometer, cytospun onto slides and identified by Wright staining.

Statistical Analysis

Mean staining intensities (Mean±SD) between FPNRBCs and AARBCs were compared using Mann-Whitney U test (GraphPad Prism software, GraphPad Prism Inc, CA). Differences were considered significant when P values were <0.05.

FPNRBC Membrane Proteins

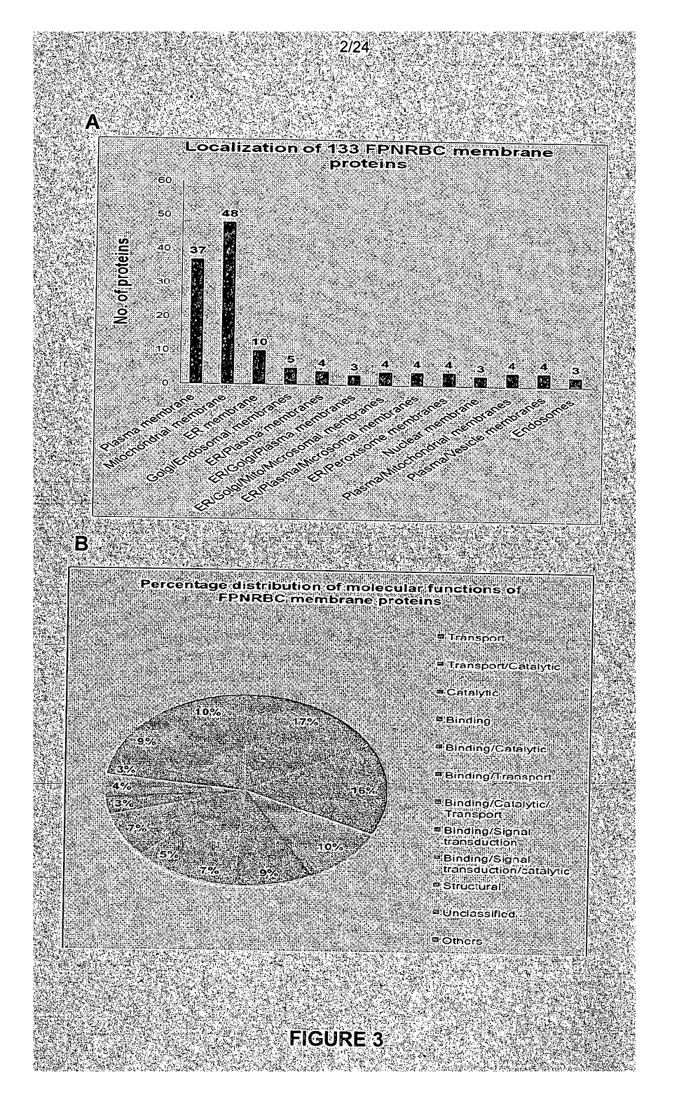

Cell membrane protein extraction is challenging because many of these proteins have hydrophobic side chains. Furthermore, the significant quantity of protein needed for detailed proteomic analysis restricts studies on limited-access cells such as the human FPNRBCs. To overcome these difficulties, cell membrane protein material harvested from several trophoblastic villi were collected and pooled, and developed a protocol for maximal cell membrane protein recovery. Two organic solvents, MeOH and TFE, were used and recovered both hydrophilic and hydrophobic proteins using pooled samples of FPNRBCS. A total of 273 proteins were identified, with 144 recovered in MeOH and 199 proteins recovered in TFE digests respectively, while 70 proteins were common to both (Table 2; FIG. 2). Only 26% of total proteins identified were recovered from both the solvents. The recovery of proteins may be enhanced by the sequential use of both solvents with limited sample (5×10⁷ cells).

As FPNRBCs are nucleated, and also contain other organelles, protein identification found not only plasma membrane proteins, but also membrane proteins from the nucleus, mitochondria, endoplasmic reticulum, Golgi, microsomes and peroxisomes.

Location Annotation of Identified Proteins

A total of 273 proteins were identified, and their locations within the cell annotated (Table 3): 133 were membrane proteins (Table 3) while 132 were non-membrane proteins including 16 that have been described as exclusively cytoplamic (Table 4). Locations of the remaining 8 are as yet unclassified (Table 5).

Sub-cellular localization and functional categories of the identified proteins were obtained based on the annotations of Gene Ontology using GoFig. (http://udgenome.ags.udel.edu/gofigure/index.html). Swiss-prot and TrEMBL data base were also used for the functional annotations of unique proteins of FPNRBCs. Sub-cellular localizations of the 133 membrane proteins were analyzed: of these proteins, 37 were noted to localize to the plasma membrane, 48 mitochondrial membranes, 10 endoplasmic reticular membranes, and the remaining 38 membrane proteins were annotated to be localized in more than one location of the cell (FIG. 3A).

Functional Annotation of Membrane Proteins

Molecular functions of the 133 membrane proteins identified are detailed in the FIG. 3B. Some proteins were noted to have more than one function. Most were transport proteins (16.54%), 15.79% were both transport and catalytic, 9.77% catalytic, 9.02% binding, 6.77% binding and catalytic, 5.26% binding and transport, 7.51% binding/catalytic/transport, 3.76% binding/signal transduction/catalytic, 3.00% each for binding/signal transduction, and structural, 9.02% unclassified and 10.53% other functions.

Proteins with Transmembrane Domains

Transmembrane domains (TMDs) of all the proteins are provided in the Table 2. The number of predicted transmembrane domains in the identified membrane proteins varied from 0 to 15: NADH dehydrogenase subunit 5 was found to possess the maximum number of TMD. Plasma membrane proteins of primitive FPNRBCs with at least one TMD (25 proteins) and the plasma membrane proteins known to be present on other membranes as well (14 proteins) are presented in Tables 6 and 7, respectively.

TABLE 2
Total proteins identified in FPNRBCs

| | | | | | | TFE | | MeOH | | | |
|---|---|---|---|---|---|---|---|---|---|---|---|
| | | | Protein | Protein | TMH [symbol] | Peptide | Best Ion | Peptide | Best Ion | | |
| | Accession # | Protein description | MW | PI | (V2) | Count | Score | Count | Score | Subcellular location | Molecular function |
| 1 | IPI00022381 | Band 3 anion transport protein | 101727.41 | 5.03 | 11 | 15 | 149 | 11 | 178 | Plasma membrane | Transporter activity |
| 2 | IPI00453473 | Histone H4 | 11229.34 | 11.36 | 0 | 12 | 122 | 11 | 116 | Nuclear | Binding |
| 3 | IPI00217471 | Hemoglobin epsilon chain | 10061.43 | 6.68 | 0 | 5 | 104 | 6 | 170 | Cytoplasmic | Transporter activity |
| 4 | IPI00152765 | Histone H2B n | 13766.52 | 10.32 | 0 | 0 | 146 | 9 | 128 | Nuclear | Binding |
| 5 | IPI00291467 | ADP/ATP translocase 3 | 32714.15 | 9.76 | 2 | 6 | 97 | 5 | 102 | Mitochondrial inner membrane | Binding: Transporter activity |
| 6 | IPI00375676 | Ferretin light chain | 28399.25 | 6 | 0 | 4 | 134 | 3 | 104 | Cytoplasmic | Binding (Iron) |
| 7 | IPI00220194 | Solute carrier family 2, facilitated glucose | 54082.52 | 8.93 | 12 | 6 | 83 | 6 | 92 | Plamsa membrane | Transporter activity |
| | | transporter member 1 | | | | | | | | | |
| 8 | IPI00022462 | Transferrin receptor protein 1 | 84547.95 | 8.18 | 1 | 5 | 96 | 2 | 55 | Plasma membrane | Receptor activity (signling), |
| | | | | | | | | | | | Catalytic acitivity |
| 9 | IPI00305383 | Uniquinol cytochrome-c reductase complex | 48412.88 | 8.74 | 0 | 4 | 129 | 2 | 127 | Mitochondrial inner membrane | Catalytic |
| | | core protein 2 mirochrondrial precursor | | | | | | | | | |
| 10 | IPI00020984 | Catherin precursor | 67525.85 | 4.47 | 1 | 5 | 103 | 4 | 89 | ER membrane | Binding |
| 11 | IPI00646289 | 25 kDa protein | 25141.15 | 8.03 | 1 | 4 | 113 | 3 | 77 | Unclassified | Unclassified |
| 12 | IPI00028014 | Splice Isoform Short of Erythrocyte membrane | 70793.61 | 8.27 | 0 | 4 | 100 | 4 | 89 | Plasma membrane | Structural molecular activity |
| | | protein band 4 2 | | | | | | | | | |
| 13 | IPI00218448 | Histine H2A [symbol] | 13413.51 | 10.58 | 0 | 4 | 87 | 4 | 83 | Nuclear | Binding |
| 14 | IPI00219038 | H3 histone Family 3B | 15318.50 | 11.27 | 0 | 5 | 85 | 5 | 147 | Nuclear | Binding |
| 15 | IPI00215777 | Splice Isoform B of Phosphate carrier protein, | 39932.64 | 9.43 | 2 | 4 | 111 | 1 | 41 | Mitochondrial inner membrane | Transporter activity |
| | | mitochondrial precursor | | | | | | | | | |
| 16 | IPI00646240 | Hypothetical protein | 7390.90 | 8.86 | 0 | 3 | 112 | 4 | 68 | Nuclear | Binding |
| 17 | IPI00470674 | NAD(P)H quinone oxidoreductase type 3, | 34073.18 | 9.41 | 1 | 4 | 98 | 3 | 106 | ER Membrane: mitochondrial | Catalytic activity: Tranporter |
| | | polypeptide A2 variant | | | | | | | | outer membrane | |
| 18 | IPI00236554 | Splice Isoform H14 of M [symbol] precursor | 73808.61 | 9.3 | 0 | 4 | 79 | 7 | 77 | Lysocome: Nuclear | Binding Catalytic Tranporter: |
| | | | | | | | | | | | Antioxidant |
| 19 | IPI00013415 | 40S ribosomal protein S7 | 22110.26 | 10.09 | 0 | 3 | 100 | 6 | 89 | Rubosomal | Structural molecular activity |
| 20 | IPI00047085 | Ribosomal protein L5 variant | 34340.69 | 9.73 | 0 | 3 | 131 | 5 | 72 | Rubosomal | Structural molecular activity |
| 21 | IPI00025038 | [symbol] | 32753.42 | 10.18 | 0 | | 94 | 6 | 72 | NuclearNuclear | Binding (RNA) |
| 22 | IPI00003968 | NADH-ubiquinone oxidoreductase 39 kDa subunit, | 42462.57 | 9.81 | 0 | 3 | 100 | 2 | 91 | Mitochondrial | Catalytic activity, Transporter |
| | | mitochondrial precursor | | | | | | | | | |
| 23 | IPI00176629 | PREDICTED similar to ribosomal protein L18a | 20753.89 | 10.73 | 0 | 4 | 71 | 4 | 49 | Ribosomal | Structural molecular activity |
| 24 | IPI00027270 | 003 ribosomal protein L26 | 17247.63 | 10.55 | 0 | 3 | 84 | 1 | 44 | Ribosomal | Structural molecular activity |
| 25 | IPI00454695 | [symbol] variant | 21458.18 | 10.71 | 0 | 3 | 84 | 4 | 83 | Nuclear | Binding |
| 26 | IPI00003057 | [symbol] , mitochondrial precursor | 50119.97 | 8.59 | 0 | 4 | 56 | 3 | 59 | Mitochondrial | Catalytic activity |
| 27 | IPI00386491 | Splice Isoform Short of [symbol] nuclear | 88890.16 | 5.6 | 0 | 3 | 86 | 6 | 123 | Nuclear | Binding |
| | | ribonucleoprotein U | | | | | | | | | |
| 28 | IPI00025086 | Cylochrome c oxidase polypeptide Va, | 16763.72 | 6.3 | 0 | 2 | 85 | 2 | 56 | Mitochondrial inner membrane | Transporter activity |
| | | milochondial precursor | | | | | | | | | |
| 29 | IPI00645733 | Lamin B receptor variant | 70051.06 | 9.41 | 8 | 3 | 85 | 3 | 67 | Nuclear inner membrane | binding |
| 30 | IPI00328416 | NADH-cylochrome b5 reductase | 34081.68 | 7.31 | 0 | 3 | 86 | 2 | 76 | ER Membrane: Mitochondrial | Catalytic activity, Transporter |
| | | | | | | | | | | outer membrane | |
| 31 | IPI00552125 | HNRPC protein | 27604.40 | 4.55 | 0 | 2 | 60 | 1 | 59 | Nuclear | Binding |
| 32 | IPI00554464 | Solute carrier family 3 (activatorS of dbaSe and | 71079.20 | 4.84 | 1 | 2 | 85 | 4 | 64 | Plasma membrane | Transporter activity |
| | | neutral amino acid tranSport) [symbol] | | | | | | | | | |
| 33 | IPI00405442 | ATP blinding cassette half-transporter | 99649.17 | 9.26 | 9 | 3 | 52 | 2 | 59 | Plasma membrane | Catalytic activity |
| 34 | IPI00550302 | Equilibrative nucleoside transporter 1 | 58824.58 | 8.49 | 11 | 2 | 79 | 1 | 52 | Plasma membrane | Transporter activity |
| 35 | IPI00219729 | Mitochondrial 2-oxoglutarate [symbol] carrier protein | 33003.81 | 9.92 | 0 | 2 | 84 | 1 | 61 | Mitochondrial membrane | Binding: Transporter activity |
| 36 | IPI00411037 | Nuclear protein Nop56 | 66194.78 | 8.21 | 0 | 2 | 86 | 3 | 61 | Nuclear | Chaperone |
| 37 | IPI00029264 | Cytochrome c1, nomo protein milochondial precursor | 35367.00 | 9.15 | 0 | 2 | 110 | 1 | 75 | Mirochondrial membrane | Transporter activity |
| 38 | IPI00219155 | 60S ribosomal protein I.27 | 15656.71 | 10.56 | 0 | 2 | 68 | 3 | 63 | Ribosomal | Structural molecular activity |
| 39 | IPI00456758 | Ribosomal protein L27o | 16468.03 | 11 | 0 | 2 | 76 | 2 | 93 | Ribosomal | Structural molecular activity |
| 40 | IPI00013847 | Ubiquinol-cylochrome-c reductase complex core | 52585.42 | 5.84 | 0 | 2 | 48 | 4 | 58 | Mitochondrial inner membrane | Catalytic activity, Transporter |
| | | protein I mitochondrial precursor | | | | | | | | | |
| 41 | IPI00550021 | 60S ribosomal protein I.3 | 45948.72 | 10.19 | 0 | 2 | 73 | 6 | 96 | Ribosomal | Structural molecular activity |
| 42 | IPI00220459 | [symbol] bloob group glycoprotein | 82770.92 | 8.09 | 1 | 1 | 53 | 2 | 93 | Plasma membrane | Catalytic activity, binding |
| 43 | IPI00217030 | 40S ribosomal protein S4. X isoform | 29448.01 | 10.16 | 0 | 2 | 73 | 2 | 50 | Ribosomal | Structural molecular activity |
| 44 | IPI00220410 | Ubiquemol-cytochrome c reductase complex | 13390.84 | 8.75 | 0 | 2 | 59 | 2 | 61 | Mitochondrial inner membrane | Catalytic activity, Transporter |
| | | 12 kDa protein | | | | | | | | | |
| 45 | IPI00020021 | DEK protein | 42647.92 | 8.69 | 0 | 1 | 49 | 3 | 53 | Nuclear | Binding: Trancription regulation |
| | | | | | | | | | | | activity |
| 46 | IPI00037070 | Splice Isoform 2 of Heat shock cognate 71 kDa protein | 53867.70 | 5.74 | 0 | 2 | 51 | 2 | 46 | Cytoplasmic nuclear | Chaperone: binding |
| 47 | IPI00002372 | ATP-binding casstte sub-family D member 3 | 75427.57 | 8.41 | | 1 | 44 | 2 | 64 | [symbol] membrane | Catalytic activity [symbol] |
| 48 | IPI00027769 | [symbol] precursor | 28499.79 | 9.71 | 1 | 1 | 123 | 1 | 101 | Plasma membrane (Estracellular) | Catalytic activity |
| 49 | IPI00026111 | Membrane protein | 21161.16 | 9.77 | 2 | 1 | 110 | 1 | 59 | ER and Golgi appartus membrane | Unclassified |
| 50 | IPI00027180 | CAAX prenyl protease 1 homolog | 54777.53 | 7.12 | 7 | 1 | 97 | 1 | 93 | ER membrane: Golgi Plasma | Catalytic activity |
| | | | | | | | | | | membrane | |
| 51 | IPI00005202 | Membrane associated progesteron receptor | 23803.73 | 4.70 | 1 | 1 | 94 | 1 | 44 | Mitochondrial membrane | Signal transducer (receptor |
| | | component 2 | | | | | | | | | activity) binding |
| 52 | IPI00219486 | Splice Isofrom 2 of 40S ribosomal protein S24 | 15059.24 | 10.69 | 0 | 1 | 81 | 1 | 79 | Ribosomal | Structural molecular activity |
| 53 | IPI00046848 | Growth-initiating protein 12 | 78334.00 | 8.54 | 0 | 1 | 81 | 1 | 84 | Plasma membrane (Extracellular) | Catalytic activity: binding |
| 54 | IPI00395887 | Thioredoxin domain containing protein 1 precursor | 31770.80 | 4.92 | 3 | 1 | 85 | 1 | 65 | ER membrane | Transporter activity |
| 55 | IPI00027448 | ATP synthase beta chain milochondial | 56524.60 | 5.26 | 0 | 1 | 82 | 1 | 49 | Milochondial outer membrane | Binding: Catalytic activity |
| 56 | IPI00024742 | Ubiquinol-cytochrome c reductase complex | 6769.08 | 10.08 | 0 | 1 | 82 | 1 | 42 | Milochondial inner membrane | Transporter: Catalytic activity |
| | | ubiquinon-binding protein QP-C | | | | | | | | | |
| 57 | IPI00182533 | 60S ribosomal protein 128 | 15606.63 | 12.02 | 0 | 1 | 81 | 1 | 74 | Ribosomal | Structural molecular activity |
| 58 | IPI00646415 | RAB14 member RAS oncogene family | 20396.31 | 5.94 | 0 | 1 | 78 | 1 | 38 | Unversal | Binding: catalytic |
| 59 | IPI00022092 | Brain Protein 44 | 11573.18 | 10.21 | 0 | 1 | 74 | 1 | 62 | Unclassifed | Transporter activity: Binding |
| 60 | IPI00028064 | [symbol] G precursor | 28819.07 | 11.19 | 01 | 1 | 73 | 1 | 45 | Plasma Membrane (associated | Catalytic activity |
| | | | | | | | | | | intermediate) | |
| 61 | IPI00100247 | Thioredoxin-like protein KIAA1162 precursor | 38927.68 | 4.31 | 0 | 1 | 70 | 1 | 90 | Plasma membrane (associated) | Transporter activity |
| 62 | IPI00010740 | Splice Isoform long of Splicing factor, proline and | 72217.75 | 0.26 | 0 | 1 | 64 | 1 | 65 | Nuclear | Binding |
| | | glutamine-rich | | | | | | | | | |
| 63 | IPI00465315 | Cytochrome c | 11510.09 | 9.59 | 0 | 1 | 62 | 1 | 38 | Milochondial inner membrane | Transporter activity |
| 64 | IPI00440703 | GSTK1 protein | 31545.51 | 8.85 | 0 | 1 | 61 | 1 | 75 | Milochondial | Catalytic: Binding |
| 65 | IPI00396485 | 50 kDa protein | 50138.50 | 7.20 | 0 | 1 | 60 | 1 | 54 | Nuclear Cytoplasmic | Binding [symbol] catalytic activity |
| 66 | IPI00021924 | Histone H1x | 22473.53 | 10.76 | 0 | 1 | 52 | 1 | 79 | Nuclear | Binding |
| 67 | IPI00552514 | Splice Isoform 1 of Vacoular [symbol] translocating | 96350.11 | 6.02 | 7 | 1 | 47 | 1 | 54 | Vesicle membrane: Plasma membrane | Catalytic activity: Transpoter |
| | | ATPase 118 kDa subunit a isoform 1 | | | | | | | | | |
| 68 | IPI00168981 | [symbol] receptor 1 [symbol] | 36861.18 | 8.05 | 7 | 1 | 47 | 1 | 39 | Plasma membrane | Receptor activity |
| 69 | IPI00001100 | CDNA PSEC0252 [symbol] clone NT2RP3003258 highly | 5494.25 | 8.71 | 11 | 1 | 47 | 1 | 54 | ER membrane protein: Plasma | Unclassified |
| | | similar to [symbol] ortholog of mouse [symbol] | | | | | | | | membrane | |
| 70 | IPI00007428 | PRA1 family protein 3 | 21500.41 | 9.77 | 3 | 1 | 43 | 1 | 49 | Plasma membrane: ER membrane | Binding |
| 71 | IPI00216697 | [symbol] 1 isoform 1 | 206128.92 | 5.85 | 0 | 9 | 104 | | | Plasma membrane | Structural molecular activity |
| 72 | IPI00639812 | Milochondial [symbol] 3 | 18484.62 | 9.99 | 3 | 4 | 117 | | | Microsome membrane: ER | Catalytic activity |
| 73 | IPI00220855 | H2A histone family member isoform 2 | 14010.83 | 10.9 | 0 | 7 | 132 | | | Nuclear | Binding |
| 74 | IPI00339774 | Histine H2A.q | 13848.80 | 10.9 | 0 | 7 | 121 | | | Nuclear | Binding |
| 75 | IPI00007188 | ADP/ATP Translocase 2 | 32743.13 | 9.76 | 2 | 7 | 131 | | | Milochondial inner membrane | Binding: Transporter activity |
| 76 | IPI00015826 | ATP binding cassette sub-family B member | 79048.95 | 9.91 | 5 | 5 | 72 | | | Milochondial inner membrane | Binding: Catalytic activity |
| | | milochondial precursor | | | | | | | | | |
| 77 | IPI00027252 | B-cell receptor-associated protein BAP37 | 33275.92 | 9.83 | 0 | 5 | 56 | | | Milochondial membrane | Signal transduce: receptor |
| | | | | | | | | | | | (binding) |
| 78 | IPI00549250 | HP1-BP74 | 61159.27 | 9.69 | 0 | 3 | 111 | | | Nuclear | Binding |
| 79 | IPI00412713 | [symbol] protein CGI-51 | 51928.80 | 6.44 | 0 | 3 | 93 | | | Milochondial Outer membrane | Tranporter: Catalytic activity: |
| | | | | | | | | | | | binding |
| 80 | IPI00216587 | 40S ribosomal protein S0 | 24009.12 | 10.02 | 0 | 4 | 88 | | | Ribosomal | Structural molecular activity |
| 81 | IPI00025874 | [symbol] protein [symbol] 67 kDa subunit precursor | 62526.81 | 5.96 | 1 | 2 | 93 | | | ER membrane | Catalytic activity: binding |
| 82 | IPI00011654 | Tabulin beta-2 chain | 48638.97 | 4.78 | 0 | 2 | 95 | | | Cytoplasmic (cytoskeleton) | Signal transducer: Structural |
| | | | | | | | | | | | molecular |
| | | | | | | | | | | | activity: binding: chaperone |
| 83 | IPI00470829 | Splice Isoform 3 of Milochondial inner membrane | 79977.51 | 6.31 | 0 | 2 | 69 | | | Milochondial inner membrane | Structural molecular activity: |
| | | protein (Proliferation inducing gene 4 [symbol] | | | | | | | | | binding: [symbol] |
| 84 | IPI00031357 | [symbol] oxidase | 50733.65 | 6.44 | 0 | 2 | 81 | | | Milochondial | Catalytic activity: Transporter |
| 85 | IPI00294778 | Splice Isoform 7 of Voltage-dependent anion- | 30770.33 | 8.85 | 0 | 2 | 100 | | | Milochondial outer membrane | Transporter activity |
| | | selective channel protein 3 | | | | | | | | | |
| 86 | IPI00472119 | PREDICTED: similar to ribosomal protein SSa | 29951.83 | 8.78 | 0 | 2 | 64 | | | Ribosomal | Structural molecular activity |
| 87 | IPI00440498 | ATP synthase alpha chain milochondial precursor | 59713.59 | 9.18 | 0 | 2 | 69 | | | Milochondial membrane | Transporter activity: binding: |
| | | | | | | | | | | | catalytic activity |
| 88 | IPI00334432 | 16 kDa protein | 15032.11 | 8.78 | 0 | 3 | 144 | | | Cytoplasmic | Transporter activity |
| 89 | IPI00456746 | ATP synthase H+ transporting milochondial F0 | 17451.13 | 9.36 | 0 | 2 | 95 | | | Milochondial | Transporter activity |
| | | complex subunit to isoform 2 | | | | | | | | | |
| 90 | IPI00383296 | Heterogeneous nulear [symbol] isoform b | 73572.38 | 8.94 | 0 | 2 | 100 | | | Nuclear | Binding |
| 91 | IPI00363240 | Milochondial substrate carrier family protein | 28874.04 | 9.66 | 0 | 2 | 59 | | | Milochondial inner membrane | Binding |
| 92 | IPI00339385 | Splice isoform 2 of Retinol dehydorgenase 11 | 33981.88 | 8.95 | 0 | 2 | 101 | | | ER membrane | Catalytic activity |
| 93 | IPI00555878 | Probable DNA [symbol] editing enzyme [symbol] | 22010.96 | 7.62 | 0 | 2 | 79 | | | Unclassified | Catalytic activity: binding |
| 94 | IPI00641334 | Similar to Cytochrome b5 outer milochondial | 14163.00 | 5.06 | 0 | 2 | 86 | | | Milochondial outer membrane | Unclassified |
| | | membrane isoform precursor | | | | | | | | | |
| 95 | IPI00096988 | Splice Isoform A of protein C20orf108 | 20411.22 | 10.45 | 3 | 2 | 89 | | | ER membrane: milochondial: | Transporter activity: Catalytic |
| | | | | | | | | | | microsome | activity |
| 96 | IPI00009346 | Transmembrane protein 14C | 1158.97 | 9.8 | 4 | 2 | 83 | | | ER membrane: Golgi membrane | unknown |
| 97 | IPI00639810 | Tricarboxytate transport protein milochondial | 35972.12 | 10.12 | 0 | 3 | 60 | | | Milochondial inner membrane | Binding: Transporter activity |
| | | precursor | | | | | | | | | |
| 98 | IPI00025796 | NADH-ubiquinone oxidoreductase 30 kDa subunit | 30222.71 | 6.99 | 0 | 2 | 74 | | | Milochondial inner membrane | Catalytic activity: transporter |
| | | milochondial precursor | | | | | | | | | activity: binding |
| 99 | IPI00398234 | 21 kDa protein | 20880.74 | 9.46 | 0 | 2 | 60 | | | Ribosomal | Structural molecular activity: |
| | | | | | | | | | | | binding |
| 100 | IPI00455155 | Rhesus blood group [symbol] antigens isoform 1 | 45421.20 | 9.4 | 12 | 2 | 61 | | | Plasma membrane | Transorter activity |
| 101 | IPI00003833 | Milochondial carrier homolog 2 | 33308.80 | 8.25 | 0 | 2 | 55 | | | Milochondial inner membrane | Binding |
| 102 | IPI00 [symbol] | 40S ribosomal protein S11 | 18418.99 | 10.31 | 0 | 2 | 60 | | | Ribosomal | Sructural molecular activity |
| 103 | IPI00 [symbol] | [symbol] precursor | 27789.27 | 8.72 | 0 | 2 | 52 | | | Plasma membrane (Exxtracellular) | Catalytic activity: binding: |
| | | | | | | | | | | cytoplasmic | signal transducer |
| 104 | IPI00 [symbol] | [symbol] protein | 15095.98 | 7.9 | 0 | 4 | 134 | | | Cytoplasmic (homoglobin complex) | Transporter activity |
| 105 | IPI00644458 | SM-11044 binding protein | 29916.72 | 5.52 | 1 | 2 | 48 | | | Endosomal membrane | Transporter |
| 106 | IPI00010746 | [symbol] synthase 1 | 55491.10 | 8.71 | 9 | 2 | 33 | | | Milochondial membrane | Catalytic activity |
| 107 | IPI00217169 | Splice Isoform XB of Plasma membrane calcium- | 133845.70 | 6.04 | 8 | 2 | 41 | | | Plasma membrane | Binding: catalytic activity |
| | | transporting ATPase 4 | | | | | | | | | transporter |
| 108 | IPI00107750 | Optic [symbol] 1 isoform 5 | 113445.91 | 7.63 | 0 | 2 | 43 | | | Milochondial | Motor: bnding: catalytic activity |
| 109 | IPI00471915 | 48 kDa protein | 47530.39 | 11.07 | 0 | 2 | 52 | | | Ribosomal | Structural molecular activity: |
| | | | | | | | | | | | binding |
| 110 | IPI00395769 | Splice Isoform Heart of ATP synthase gamma | 32860.24 | 0.31 | 0 | 2 | 55 | | | Milochondial inner membrane | Tranposter activity: Catalytic |
| | | chain milochondial precursor | | | | | | | | | activity |
| 111 | IPI00293073 | Milochondial tranmembrane GTPase FZO-2 | 86938.53 | 5.99 | 0 | 2 | 61 | | | Milochondial membrane | Binding: catalytic activity |
| 112 | IPI00456049 | ATP synthase H+ transporting milochondial F0 | 5763.17 | 6.6 | 0 | 2 | 57 | | | Milochondial | Transporter activity |
| | | complex, subunit d isoform b | | | | | | | | | |
| 113 | IPI00452747 | KIAAD102 protein | 14159.28 | 7.85 | 1 | 2 | 38 | | | ER membrane: Microsome membrane | Catalytic activity |
| 114 | IPI00639942 | Ribosomal pRotein S29 isofoRm 2 | 8081.99 | 10.08 | 0 | 1 | 39 | | | Ribosomal | Structural molecular activity |
| 115 | IPI00186338 | 10 kDa protein | 9733.91 | 5.81 | 0 | 1 | 90 | | | Nuclear | Binding (DNA) |
| 116 | IPI00642216 | 17 kDa protein | 16710.62 | 6.49 | 3 | 1 | 92 | | | Plasma membrane: ER membrane | Tranporter activity: Catalytic |
| | | | | | | | | | | | activity: signal transducer |
| | | | | | | | | | | | activity (receptor) |
| 117 | IPI00017510 | Cytochrome c oxidase subunit 2 | 25548.21 | 4.67 | 2 | 1 | 90 | | | Milochondial inner membrane | Tranposter activity: binding: |
| | | | | | | | | | | | catalytic activity |
| 118 | IPI00007676 | Steroid dehydrogenase homolog | 34328.24 | 9.34 | 3 | 1 | 87 | | | ER membrane: multipass memb | catalytic activity |
| 119 | IPI00016342 | Ras-related protein Rab-7 | 23474.84 | 8.4 | 0 | 1 | 70 | | | Endosome: Golgi membrane | Catalytic activity: Transporter: |
| | | | | | | | | | | | binding |
| 120 | IPI00216115 | Splice Isoform GN 1S of Glycogenin 1 | 28090.35 | 4.73 | 0 | 1 | 78 | | | Cytoplasmic | Catalytic activity |
| 121 | IPI00478327 | [symbol] | 22617.43 | 10.43 | 0 | 1 | 67 | | | Ribosomal | Structural molecular activity: |
| | | | | | | | | | | | banding |
| 122 | IPI00554589 | Hypothetical protein FLJ35097 | 48583.91 | 8.96 | 0 | 1 | 133 | | | Milochondial | catalytic activity: binding |
| 123 | IPI00221092 | 40S ribosomal protein S16 | 16304.00 | 10.21 | 0 | 1 | 75 | | | Ribosomal | Structural molecular activity |
| 124 | IPI00172656 | Protein expressed in T-cells and [symbol] in [symbol] dermatitis | 52590.54 | 5.48 | 0 | 1 | 73 | | | Nuclear: Cytoplasmic | Unknown |
| 125 | IPI00022246 | [symbol] precursor | 26868.65 | 9.75 | 0 | 1 | 75 | | | Plasma membrane (Extracellular): | Catalytic activity binding: signal |
| | | | | | | | | | | cytoplasmic | transducer |
| 126 | IPI00106079 | Vitamin k epoxide reduxtase omplex subunit 1-like | 19822.68 | 9.28 | 2 | 1 | 73 | | | Multipass membrane protein | Unclassified |
| | | protein 1 | | | | | | | | (potential) | |
| 127 | IPI00008167 | Soduim/potassium-transporting ATPase beta-3 chain | 31492.09 | 8.56 | 1 | 1 | 71 | | | Plasma membrane | Transporter adhesion: catalytic |
| | | | | | | | | | | | activity |
| 128 | IPI00215610 | 55 kDa erythtocyte membrane protein | 52263.65 | 6.91 | 0 | 1 | 70 | | | Plasma membrane | catalytic activity (binding) |
| 129 | IPI00644559 | 12 kDa protein | 12027.62 | 6.81 | 0 | 1 | 70 | | | Milochondial | Transporter catalytic activity |
| 130 | IPI00643648 | NADH dehydrogenase | 52526.70 | 7.21 | 0 | 1 | 89 | | | Milochondial inner membrane | |
| 131 | IPI00455976 | PREDICTED: similar to hypothetical protein | 27850.92 | 10.36 | 0 | 1 | 69 | | | Ribosomal | Structural molecular activity |
| 132 | IPI00023510 | Ras-related protein Rab-5A | 23643.82 | 6.32 | 0 | 1 | 69 | | | Endosome: Golgi membrane | Catalytic activity: Transporter: |
| | | | | | | | | | | | binding |
| 133 | IPI00019472 | Neutral amino acid transporter B | 58576.27 | 5.34 | 0 | 1 | 68 | | | Plasma membrane | Signal transducer (receptor |
| | | | | | | | | | | | activity): Transporter |
| 134 | IPI00184474 | Splice Isoform 3 of protein GPR107 precursor | 61936.41 | 6.72 | 7 | 1 | 68 | | | Plasma membrane | Signal ransducer: catalytic: |
| | | | | | | | | | | | binding |
| 135 | IPI00219037 | Hogh mobility group protein 2 | 23887.68 | 7.77 | 0 | 1 | 67 | | | Nuclear | binding: transcription: enzyme |
| | | | | | | | | | | | regulator activity |
| 136 | IPI00007067 | Golgi-associated plant pathogenesis related protein 1 | 17078.48 | 9.44 | 0 | 1 | 66 | | | Golgi membrane: extracellular | Unclassified |
| 137 | IPI00016513 | Ras-related protein Rab-10 | 22528.59 | 8.59 | 0 | 1 | 65 | | | Endosome: Golgi membrane | Catalytic activity: Transporter: |
| | | | | | | | | | | | binding |
| 138 | IPI00374181 | ATP synthase F0 subunit 6 | 24735.88 | 10.09 | 6 | 1 | 69 | | | Milochondial inner membrane | Transporter activity |
| 139 | IPI00104050 | Thyroid hormone receptor-associated protein complex | 108629.04 | 10.16 | 0 | 1 | 65 | | | Nuclear | Transcription signal transducer: |
| | | 150 kDa component | | | | | | | | | binding |
| 140 | IPI00335277 | Splice Isoform 2 of Synaptophysin-like protein | 26394.49 | 6.79 | 3 | 1 | 64 | | | Vesicle membrane: plasma membrane | Transporter activity: Binding |
| 141 | IPI00007755 | Ras-related protein Rab-21 | 24201.21 | 8.16 | 0 | 1 | 62 | | | Endosome: Golgi membrane [symbol] | Catalytic activity: Transporter: |
| | | | | | | | | | | | binding |
| 142 | IPI00376215 | Splice Isoform 2 of DNA-dependent protein kinase | 465202.00 | 6.81 | 0 | 1 | 54 | | | Nuclear | catalytic activity |
| | | catalytic subunit | | | | | | | | | |
| 143 | IPI00447606 | SLC27A2 protein | 64574.28 | 6.73 | 1 | 1 | 61 | | | ER membrane: [symbol] membrane | catalytic activity |
| 144 | IPI00414695 | PREDICTED: similar to ribosomal protein S2 | 29940.03 | 10.01 | 0 | 1 | 61 | | | Ribosomal | Structural molecular activity: |
| | | | | | | | | | | | binding |
| 145 | IPI00031064 | Hypothetical protein DKFZp586C1924 | 21413.48 | 9.36 | 2 | 1 | 61 | | | Multipass membrane protein | Unclassified |
| | | | | | | | | | | (potential) | |
| 146 | IPI00414000 | Hypothetical protein DKFZp686L18234 | 37566.79 | 9.45 | 0 | 1 | 59 | | | Multipass membrane protein | catalytic activity |
| | | | | | | | | | | (potential) | |
| 147 | IPI00032831 | [symbol] -associated protein 29 | 28952.59 | 5.56 | 0 | 1 | 59 | | | Plasma membrane: [symbol] | Transporter |
| 148 | IPI00031691 | 60S ribosomal protein L9 | 21849.80 | 9.96 | 0 | 1 | 58 | | | Ribosomal | Structural molecular activity |
| 149 | IPI00386258 | Milochondial carrier homolog 1 isoform b | 41517.29 | 9.4 | 2 | 1 | 57 | | | Milochondial membrane extracellular | binding |
| 150 | IPI00479694 | 13 kDa protein | 12579.21 | 10.85 | 0 | 1 | 87 | | | Ribosomal | Structural molecular activity |
| 151 | IPI00221298 | Splice Isoform of NADH-ubiquinone oxidoreductase | 40836.33 | 8.51 | 0 | 1 | 58 | | | Milochondial inner membrane | catalytic activity transporter |
| | | 51 kDa subunit, milochondial precursor | | | | | | | | | |
| 152 | IPI00204642 | Hypothetical protein FLJ14938 | 55638.31 | 4.78 | 0 | 1 | 56 | | | Singlepass membrane | Unclassified |
| 153 | IPI00383085 | FLJ00144 | 28437.22 | 5.75 | 0 | 1 | 50 | | | unclassified | Unclassified |
| 154 | IPI00017334 | Prohibitin | 29785.90 | 5.57 | 0 | 1 | 54 | | | Milochondial membrane | signal transducer binding |
| 155 | IPI00428490 | Aquaporin I splice varian 2 | 14913.58 | 5.48 | 1 | 1 | 53 | | | Plasma membrane | transporter activity. Structural |
| | | | | | | | | | | | molecular activity |
| 156 | IPI00034208 | BCG induced integral membrane protein BIGM103 | 49598.38 | 5.71 | 7 | 1 | 53 | | | Plasma membrane | catalytic activity transporter |
| | | | | | | | | | | | activity |
| 157 | IPI00171459 | Hypothetical protein FLJ90397 | 36994.10 | 8.88 | 0 | 1 | 52 | | | unclassified | catalytic activity |
| 158 | IPI00021439 | Actin cytoplasmic 1 | 41709.73 | 5.29 | 0 | 1 | 52 | | | Cytoplasmic | Motor: structural molecular |
| | | | | | | | | | | | activity |
| 159 | IPI00465044 | RCC2 protein | 50049.20 | 9.02 | 0 | 1 | 51 | | | Nuclear | catalytic activity binding |
| | | | | | | | | | | | enzyme regulator activity |
| 160 | IPI00020599 | [symbol] precursor | 48111.02 | 4.29 | 0 | 1 | 52 | | | Cytoplasmic | binding transcription |
| 161 | IPI00644824 | Similar in Translocon-associated protein delta | 13240.66 | 6.48 | 1 | 1 | 51 | | | ER membrane | Binding |
| | | subunit precursor | | | | | | | | | |
| 162 | IPI00299000 | Proliferation-associated 2G4, 38 kDa | 43785.20 | 6.13 | 0 | 1 | 50 | | | Nuclear | Catalytic; binding |
| 163 | IPI00419696 | ATPasg [symbol] transporting lycosomal V0 subunit a | 98015.34 | 6.18 | 0 | 1 | 49 | | | Vacuolar membrane: lyrosome | transporter activity catalytic |
| | | isoform 2 | | | | | | | | | |
| 164 | IPI00328815 | Ubiquin-specific protease 31 | 118958.15 | 5.75 | 1 | 1 | 48 | | | Nuclear membrane | catalytic activity |
| 165 | IPI00643475 | [symbol] like domain containing protein | 21238.77 | 9.69 | 4 | 1 | 48 | | | ER membrane | Unclassified |
| 166 | IPI00329745 | 130 kDa ;eucine-rich protein | 15825.66 | 5.91 | 0 | 1 | 48 | | | Nuclear: Cytoplasmic | catalytic binding |
| 167 | IPI00218848 | ATP synthase [symbol] milochondial | 7797.28 | 9.34 | 1 | 1 | 47 | | | Milochondial membrane | transporter activity |
| 168 | IPI00397441 | PREDICTED: similar to dJ75305.2 | 13749.95 | 9.36 | 0 | 1 | 47 | | | Ribosomal | Structural molecular activity: |
| | | (novel protein similar to RP517 (40s ribosomal protein | | | | | | | | | binding |
| 169 | IPI00219682 | Erythrocyte band 7 integral membrane protein | 31579.70 | 7.9 | 1 | 1 | 47 | | | Plasma membrane | binding |
| 170 | IPI00176708 | Hypothetical protein MGC 14286 | 6595.25 | 9.58 | 1 | 1 | 46 | | | unclassified | Unclassified |
| 171 | IPI00604015 | ALEX3 protein variant | 42443.90 | 8.75 | 1 | 1 | 46 | | | unclassified | Unclassified |
| 172 | IPI00619900 | Adaptor related protein complex 2 [symbol] subunit isoform b | 49357.91 | 9.57 | 0 | 1 | 46 | | | Vesicle membrane (ass Plasma | transporter |
| | | | | | | | | | | membrane): [symbol] | |
| 173 | IPI00007084 | Milochondial [symbol] glutamate carrier protein | 74256.70 | 8.79 | 0 | 1 | 45 | | | Milochondial inner membrane | transporter: binding |
| 174 | IPI00221391 | NADH dehydrogenase subunit 5 | 66968.42 | 9.14 | 15 | 1 | 45 | | | Milochondial inner membrane | transporter: catalytic activity |
| 175 | IPI00218128 | Splice Isoform Glycopharin O of Glycophorin C | 11401.61 | 4.76 | 1 | 1 | 44 | | | Plasma membrane | cell adhesion: signal transducer |
| | | | | | | | | | | | (receptor) |
| 176 | IPI00011107 | [symbol] dehydrogenase (NADP) milochondial precursor | 50870.80 | 8.88 | 0 | 1 | 44 | | | Milochondial | Catalytic activity |
| 177 | IPI00513768 | Novel protein | 52673.49 | 9.76 | 0 | 1 | 43 | | | unclassified | binding |
| 178 | IPI00376520 | 9 kDa protein | 9459.66 | 6.75 | 1 | 1 | 43 | | | Milochondial membrane | catalytic: transporter |
| 179 | IPI00005966 | 13 kDa differentiation-associated protein variant | 17073.02 | 9.63 | 0 | 1 | 44 | | | Milochondial | catalytic: transporter |
| 180 | IPI00009247 | Full-lenght cDNA clone C S0DC006YH13 of | 8809.50 | 9.94 | 1 | 1 | 41 | | | Milochondial membrane | Unclassified |
| | | Neuroblastoma of Homo sapiens | | | | | | | | | |
| 181 | IPI00028088 | Splice Isoform 1 of Heterogeneous nuclear | 38410.30 | 7.62 | 0 | 1 | 41 | | | Nuclear | binding |
| | | ribonucleoprotein [X] | | | | | | | | | |
| 182 | IPI00014168 | Protein p65 | 68575.87 | 9.87 | 0 | 1 | 41 | | | Nuclear | binding transcription signal |
| | | | | | | | | | | | transducer (receptor) |
| 183 | IPI00335130 | 46 kDa protein | 46266.42 | 7.28 | 0 | 1 | 40 | | | milochondial transcript level | Structural ,olecular activity: |
| | | | | | | | | | | | Catalytic |
| 184 | IPI00396411 | Cleft lip. and palate transmembrane protein 1 | 76048.46 | 5.66 | 5 | 1 | 40 | | | Plasma membrane | Unclassified |
| 185 | IPI00607576 | Splice Isoform 1 of Protein C9orf5 | 100881.01 | 9.03 | 14 | 1 | 40 | | | Plasma membrane | unknown |
| 186 | IPI00642244 | Novel protein | 109351.93 | 7.94 | 0 | 1 | 40 | | | ER | Unclassified |
| 187 | IPI00217653 | Milochondial ribosomal protein I 41 | 15372.98 | 9.58 | 0 | 1 | 40 | | | Ribosomal | structural activity |
| 188 | IPI00394779 | Splice Isoform 1 of Protein C20orf22 | 45068.18 | 8.87 | 1 | 1 | 40 | | | unclassified | catalytic activity |
| 189 | IPI00383231 | Kelch domain containing protein 1 | 46061.73 | 5.67 | 0 | 1 | 39 | | | Unclassified | binding: transcription |
| 190 | IPI00419579 | F450-like protein | 52399.01 | 5.84 | 0 | 1 | 39 | | | Cytoplasmic | binding catalytic |
| 191 | IPI00221002 | [symbol] membrane protein 2 | 22106.89 | 10.58 | 4 | 1 | 48 | | | [symbol] | unknown |
| 192 | IPI00216308 | Voltage-dependent anion-selective channel protein 1 | 30622.53 | 8.63 | 0 | 1 | 50 | | | Milochondial outer membrane: | transporter activity |
| | | | | | | | | | | Plasma [symbol] | |
| 193 | IPI00007928 | Pre-mRNA processing splicing factor 8 | 273426.50 | 8.95 | 0 | 1 | 40 | | | Nuclear | Binding |
| 194 | IPI00549891 | Heparan sulfate 2-O-sulfotransferase I | 41844.40 | 8.63 | 0 | 1 | 43 | | | Nuclear: golgo; integral membrane | catalytic activity |
| 195 | IPI00180121 | Splice Isoform A of Chloride channel protein 6 | 35910.30 | 8.03 | 3 | 1 | 41 | | | Plasma membrane | ion channel activity |
| 196 | IPI00022275 | Suppressor of actin 1 | 66908.01 | 6.66 | 2 | 1 | 45 | | | ER membrane: Golgi | Catalytic: Binding: |
| 197 | IPI00299928 | [symbol] nucleotide binding protein alpha 13 subunit | 44021.68 | 8.11 | 0 | 1 | 42 | | | Plasma membrane | Signal transducer (receptor) |
| | | | | | | | | | | | binding: catalytic activity |
| 198 | IPI00065287 | Hypothetical protein FLJ32930 | 85726.57 | 9.35 | 0 | 1 | 41 | | | Unclassified | Unclassified |
| 199 | IPI00100810 | HSPC051 | 14391.31 | 10.96 | 0 | 1 | 41 | | | Milochondial membrane | Catalytic |
| 200 | IPI00237806 | Splice Isoform 2 of Spectrin beta chain, erythrocyle | 242580.00 | 5.2 | 0 | | | 5 | 83 | Plasma membrane | Structural molecular activity |
| 201 | IPI00059366 | H2A histone family, member Y isoform 2 | 39159.18 | 9.89 | 0 | | | 3 | 97 | Nuclear | Binding |
| 202 | IPI00470528 | 60S ribosomal protein L15 | 24000.04 | 11.62 | 0 | | | 2 | 38 | Ribosomal | Structural molecular activity |
| 203 | IPI00554723 | 60S ribosomal protein L10 | 24429.92 | 10.11 | 0 | | | 4 | 67 | Ribosomal | Structural molecular activity: |
| | | | | | | | | | | | binding |
| 204 | IPI00216457 | Histone II2A o | 13955.85 | 10.9 | 0 | | | 7 | 125 | Nuclear | Binding |
| 205 | IPI00217405 | Histone H1.2 | 21220.71 | 10.94 | 0 | | | 5 | 111 | Nuclear | Binding |
| 206 | IPI00 | Histone H1.4 | 21720.98 | 11.63 | 0 | | | 6 | 113 | Nuclear | Binding |
| 207 | IPI00 | Histone H1.5 | 22435.43 | 10.91 | 0 | | | 4 | 112 | Nuclear | Binding |
| 208 | IPI00 | Ubiquitin and ribosomal protein S27a | 17893.44 | 9.65 | 0 | | | 4 | 159 | Ribosomal | Structural molecular activity: |
| | | | | | | | | | | | Catalytic |
| 209 | IPI00 | Histone H1.3 | 22205.29 | 110.2 | 0 | | | 5 | 103 | Nuclear | Binding |
| 210 | IPI00 | Histone H12A | 14112.93 | 11.05 | 0 | | | 7 | 121 | Nuclear | Binding |
| 211 | IPI00 | Histone H2B.1 | 13752.50 | 10.32 | 0 | | | 9 | 128 | Nuclear | Binding |
| 212 | IPI00 | Histone H1.1 | 21697.83 | 10.99 | 0 | | | 4 | 112 | Nuclear | Binding |
| 213 | IPI00 | Ribosomal protein S8 | 21866.01 | 10.37 | 0 | | | 4 | 104 | Ribosomal | Structural molecular activity |
| 214 | IPI00 | Hemoglobin gamma-2 chain | 15885.25 | 6.71 | 0 | | | 4 | 146 | Cytoplasmic | Transporter activity |
| 215 | IPI00 | Alpha 2 globin variant | 15270.94 | 8.72 | 0 | | | 4 | 105 | Cytoplasmic | Transporter activity |
| 216 | IPI00 | Hemogen | 55278.74 | 4.82 | 0 | | | 4 | 101 | Golgi membrane: Vesicle: | Structural molecular activity |
| | | | | | | | | | | Nuclear | |
| 217 | IPI00 | 60S ribosomal protein L19 | 23451.25 | 11.48 | 0 | | | 3 | 80 | Ribosomal | Structural molecular activity |
| 218 | IPI00 | HNRPR protein | 71170.40 | 8.23 | 0 | | | 4 | 73 | Nuclear | Binding (RNA) |
| 219 | IPI00 | Ribosomal protein L5 variant | 34340.00 | 9.73 | 0 | | | 4 | 67 | Ribosomal | Structural molecular activity |
| 220 | IPI00 | 60S ribosomal protein L17 | 21252.29 | 10.18 | 0 | | | 3 | 71 | Ribosomal | Structural molecular activity |
| 221 | IPI00 | OTTHUMP00000016816 | 62617.34 | 7.18 | 0 | | | 3 | 73 | Nuclear: Cytoplasmic | Binding |
| 222 | IPI00 | Splice Isoform 2 of [symbol] domain adjacent to [symbol] | 170340.30 | 8.7 | 0 | | | 3 | 55 | Nuclear | Binding Transcription activity |
| | | domain protein 1B | | | | | | | | | |
| 223 | IPI00 | ATP synthase H+ transporting milochondial F0 | 18479.50 | 5.21 | 0 | | | 2 | 57 | Milochondial membrane | Structural molecular activity: |
| | | complex subunit d isoform a | | | | | | | | | catalytic activity: transporter |
| | | | | | | | | | | | activity |
| 224 | IPI00 | Novel protein similar to histone 2. H3c | 15420.55 | 11.27 | 0 | | | 3 | 47 | Nuclear | Binding |
| 225 | IPI00 | Cytochrome c oxidase polypeptide Via liver | 10054.68 | 6.78 | 0 | | | 2 | 75 | Milochondial membrane | Transporter activity: Catalytic |
| | | milochondial presurcor | | | | | | | | | activity |
| 226 | IPI00 | ATP-dependent RNA helicase DDX18 | 75359.44 | 9.52 | 0 | | | 2 | 60 | Nuclear | Ctalytic activity: binding |
| 227 | IPI00 | 18 kDa protein | 17039.75 | 10.66 | 0 | | | 2 | 58 | Ribosomal | Structural molecular activity: |
| | | | | | | | | | | | binding |
| 228 | IPI00 | Beta-globin gene from a [symbol] patient comptate cds | 18918.59 | 6.28 | 0 | | | 2 | 50 | Cytoplasmic | Transporter activity |
| 229 | IPI00 | 60S ribosomal protein L7 | 20207.20 | 10.66 | 0 | | | 2 | 102 | Ribosomal | Structural molecular activity |
| 230 | IPI00 | Ribosomal pRotein L15: 60S Ribosomal pRotein L15 | 24071.05 | 11.62 | 0 | | | 2 | 38 | Ribosomal | Structural molecular activity |
| 231 | IPI00 | Hemoglobin delta chain | 15914.25 | 7.87 | 0 | | | 2 | 50 | Cytoplasmic | Transporter activity |
| 232 | IPI00 | Antibacterial protein Fall-39 precursor | 19578.27 | 8.48 | 0 | | | 2 | 55 | Plasma membrane (Extracellular) | Catalytic (Enzyme inhibitor |
| | | | | | | | | | | | activity) |
| 233 | IPI00 | Splice Isoform 2 of Syntaxin-7 | 27383.60 | 5.02 | 0 | | | 2 | 66 | Endosome membrane | Transporter activity |
| 234 | IPI00 | Splice Isoform Short of Prostaglandin G11 synthase | 64440.72 | 7.9 | 0 | | | 2 | 39 | Microsome Membrane: Cytoplasmic | Catalytic activity: Antioxidant |
| | | 1 percursor | | | | | | | | | |
| 235 | IPI00 | OTTHUMP00000018319 | 12179.15 | 0.3 | 0 | | | 2 | 76 | Milochondial membrane | Transporter activity: Catalytic |
| | | | | | | | | | | | activity |
| 236 | IPI00 | PREDICTED: similar to 80S ribosomal protein L21 | 10973.01 | 10.97 | 0 | | | 2 | 51 | Ribosomal | Structural molecular activity |
| 237 | IPI00 | Hypothetical protein DKFZp564K247 | 10137.19 | 9.52 | 2 | | | 1 | 100 | integral membrane protein | Unclassified |
| 238 | IPI00 | PREDICTED: similar to 60S ribosomal protein L32 | 52270.66 | 10.1 | 0 | | | 1 | 118 | Ribosomal | Structural molecular activity |
| 239 | IPI00 | 60S ribosomal protein L35 | 14411.52 | 11.04 | 0 | | | 1 | 105 | Ribosomal | Structural molecular activity |
| 240 | IPI00 | [symbol] associated actin dependent regulator of [symbol] | 121828.30 | 8.27 | 0 | | | 1 | 81 | Nuclear | Catalytic activity: binding: |
| | | subfamily [symbol] | | | | | | | | | transcription activity |
| 241 | IPI00 | Hypothetical protein FLJ32110 | 62436.95 | 9.43 | 0 | | | 1 | 73 | Nuclear: Cytoplasmic | Binding Transcription |
| 242 | IPI00 | Splice Isoform 2 of H/ACA ribosomal protein complex | 20871.40 | 10.45 | 0 | | | 1 | 71 | Nuclear | Transporter activity: Binding |
| | | subunit 1 | | | | | | | | | |
| 243 | IPI00 | 60S ribosomal protein L22 | 14646.76 | 9.22 | 0 | | | 1 | 70 | Ribosomal | Structural molecular activity |
| 244 | IPI00 | HEAT repeat containing protein | 105826.65 | 9.5 | 0 | | | 1 | 69 | Nuclear | Catalytic activity |
| 245 | IPI00 | PREDICTED: similar to 40S ribosomal protein S7 (S8) | 16401.05 | 10.05 | 0 | | | 1 | 61 | Ribosomal | Structural molecular activity |
| 246 | IPI00 | NADH-ubiquinone oxidoreductase [symbol] subunit | 8067.10 | 8.93 | 1 | | | 1 | 64 | Milochondial membrane | Catalytic activity Transporter |
| 247 | IPI00 | NADH dehydrogenase | 30141.58 | 8.26 | 0 | | | 1 | 60 | Milochondial | Catalytic: Transporter: binding |
| 248 | IPI00 | [symbol] cell-derived receptor-1 eta | 44359.50 | 8.11 | 1 | | | 1 | 59 | Plasma membrane | Receptor activity |
| 249 | IPI00 | EH domain containing protein 3 | 61857.09 | 6.06 | 0 | | | 1 | 57 | Nuclear | Binding |
| 250 | IPI00 | Protein C10orf70 | 12191.20 | 9.19 | 1 | | | 1 | 56 | Milochondial membrane | motor: Binding |
| 251 | IPI00 | Hypothetical protein FLJ31842 | 30020.92 | 9.4 | 6 | | | 1 | 57 | Plasma membrane | Unclassified |
| 252 | IPI00 | NADH-ubiquinone oxidoreductase B22 subunit | 21685.80 | 6.59 | 0 | | | 1 | 54 | Milochondial membrane | Catalytic activity: Transporter |
| 253 | IPI00 | NADH-ubiquinone oxidoreductase 23 kDa subunit | 23689.62 | 6 | 0 | | | 1 | 51 | Milochondial membrane | Catalytic activity: Transporter |
| | | milochondial precursor | | | | | | | | | |
| 254 | IPI00 | Splice Isoform A of Band 4.1-like protein 3 | 120603.14 | 5.09 | 0 | | | 1 | 49 | Plasma membrane protein: | Structural molecular activity: |
| | | | | | | | | | | cytoskeleton | binding |
| 255 | IPI00 | 65 kDa protein | 64667.14 | 9.8 | 0 | | | 1 | 47 | Nuclear | Binding (DNA) |
| 256 | IPI00 | Hypothetical protein DKFZp313B047 | 170483.09 | 7.02 | 0 | | | 1 | 45 | Cytoplasmic | Catalytic activity: binding |
| 257 | IPI00 | 8 kDa protein | 6078.35 | 9.01 | 2 | | | 1 | 45 | Unclassified | Unclassified (function unknown) |
| 258 | IPI00 | TUBA6 protein | 36624.68 | 6.2 | 0 | | | 1 | 45 | Cytoplasmic (cytoskeleton) | Structural molecular activity: |
| | | | | | | | | | | | binding |
| 259 | IPI00 | 40S ribosomal protein 519 | 15919.49 | 10.31 | 0 | | | 1 | 44 | Ribosomal | Structural molecular activity |
| 260 | IPI00479958 | Splice Isoform 2 of | 29543.15 | 8.59 | 0 | | | 1 | 38 | nucleus | Catalytic activity |
| | | N-acylneuraminate [symbol] | | | | | | | | | |
| 261 | IPI00553138 | Vesicle-associated membrane | 12509.63 | 7.82 | 1 | | | 1 | 50 | Vesicle membrane: Integral | Transporter activity |
| | | protein 2 | | | | | | | | membrane protein | |
| 262 | IPI00607534 | Splice Isoform 2 or Myo-binding | 149273.88 | 9.34 | 0 | | | 1 | 49 | Nucleus and cytoplasm | Binding: Catalytic activity |
| | | protein 1A | | | | | | | | | |
| 263 | IPI00641145 | ATPase subunit 8 | 7972.15 | 9.93 | 1 | | | 1 | 47 | Milochondiol membrane protein | Transporter activity |
| 264 | IPI00556310 | 58 kDa protein | 57968.46 | 10.11 | 0 | | | 1 | 47 | Ribosomal | Structural molecular activity |
| 265 | IPI00031804 | Splice Isoform 1 of Voltage- | 30639.28 | 8.85 | 0 | | | 1 | 46 | Milochondial outer membrane | Tranporter |
| | | dependent anion-selective | | | | | | | | | |
| | | channel protein 3 | | | | | | | | | |
| 266 | IPI00292290 | Splice Isoform long of Demalin | 45486.22 | 8.94 | 0 | | | 1 | 45 | Plasma membrane: cytoskeleton | Binding (actin) |
| 267 | IPI00549905 | ATP synthase H+ transporting | 59771.60 | 9.07 | 0 | | | 1 | 42 | Milochondial inner membrane | Transporter: Binding: |
| | | milochondial F1complex alpha | | | | | | | | | Catalytic activity |
| | | subunit isoform 6 | | | | | | | | | |
| 268 | IPI00218466 | Sec61 alpha 1 subunit | 52230.51 | 8.3 | 10 | | | 1 | 41 | ER membrane | Tranporter |
| 269 | IPI00009950 | Vesicular integral-membrane | 40203.10 | 6.46 | 1 | | | 1 | 41 | ER membrane: Golgi | Transporter activity: Binding |
| | | protein VIP36 precursor | | | | | | | | | |
| 270 | IPI00219685 | Cell death-regulatory protein | 25804.36 | 9.82 | 1 | | | 1 | 40 | Milochondial inner membrane | Catalytic: Transporter: |
| | | GRIM19 | | | | | | | | | Binding: Apoplosis |
| 271 | IPI00386255 | Pol protein | 97621.79 | 8.89 | 0 | | | 1 | 39 | Cytoplasmic | binding |
| 272 | IPI00555919 | NDUFC2 protein | 14164.40 | 9.04 | 1 | | | 1 | 36 | Milochondial inner membrane | Catalytic: Transporter: |
| 273 | IPI00021766 | Splice Isoform 1 of [symbol] | 40292.95 | 4.71 | 2 | | | 1 | 44 | Plasma membrane: ER | Binding: Signal transducer: |
| | | | | | | | | | | membrane protein | apoplosis |

Colour coding:
Red: identified by two/more peptides
Green and Black: Identified bby Single peptide
When combined: Red > Green > Black [symbol]
indicates data missing or illegible when filed

TABLE 3
Membrane proteins of FPNRBCs with known subcellular location

| | | | | | | TFE | | MeOH | | | |
|---|---|---|---|---|---|---|---|---|---|---|---|
| | | | Protein | Protein | TMH [symbol] | Peptide | Best Ion | Peptide | Best Ion | | |
| | Accession # | Protein description | MW | PI | (V2) | Count | Score | Count | Score | Subcellular location | Molecular function |
| 1 | IPI00022361 | Band 3 anion transport protein | 101727.41 | 5.03 | 11 | 15 | 149 | 13 | 178 | Plasma membrane | Transporter activity |
| 2 | IPI00216897 | Ankyrin 1 Isoform 1 | 208136.92 | 5.85 | 0 | 9 | 104 | 6 | 81 | Plasma membrane | Structural molecular activity |
| 3 | IPI00291467 | ADP/ATP tranferase 3 | 52714.15 | 9.76 | 2 | 7 | 101 | 5 | 102 | Milochondial inner membrane: | Binding: Transpoter activity |
| | | | | | | | | | | Plasma membrane | |
| 4 | IPI00220194 | Solute carrier family 2, facilitated glucose | 54082.52 | 8.93 | 12 | 6 | 83 | 8 | 92 | Plasma membrane | Transporter activity |
| | | transporter member 1 | | | | | | | | | |
| 5 | IPI00022462 | Tranferrin receptor protein 1 | 86847.98 | 6.18 | 1 | 5 | 96 | 1 | 55 | Plasma membrane | Receptor activity (signaling): |
| | | | | | | | | | | | Catalytic activity |
| 6 | IPI00305383 | Ubiquinol- cytochrome- c reduclase complex core | 48412.88 | 8.74 | 0 | 4 | 129 | 2 | 127 | Milochondial inner membrane | Catalytic |
| | | protein 2, milochondial precurser | | | | | | | | | |
| 7 | IPI00639812 | Microsomal glutathioe S-tranferase 2 | 18404.62 | 9.89 | 3 | 4 | 117 | 4 | 91 | Plasma membrane Microsome | Catalytic activity |
| | | | | | | | | | | membrane: ER | |
| 8 | IPI00020904 | Catherin precursor | 67526.85 | 4.47 | 1 | 5 | 103 | 4 | 89 | ER membrane: Plasma membrane | Binding |
| 9 | IPI00008614 | Splice Isoform Short of Erythrocyle membrane | 76783.61 | 8.27 | 0 | 4 | 100 | 4 | 89 | Plasma membrane | Structural molecular activity |
| | | protein band 4 2 | | | | | | | | | |
| 10 | IPI00215777 | Splice Isoform B of Phosphate carrie protein, | 39932.64 | 9.43 | 2 | 4 | 111 | 1 | 41 | Milochondial inner membrane | Transporter activity |
| | | milochondial precursor | | | | | | | | | |
| 11 | IPI00470674 | NAD(P)H quinone oxidoreduclase type 3, | 34073.18 | 9.41 | 1 | 4 | 98 | 3 | 108 | ER Membrane: milochondial | Catalytic activity: Transporter |
| | | polypeptide A2 variant | | | | | | | | outer membrane | |
| 12 | IPI00025086 | Cytochrome c aditase polypeptide VA. | 16763.72 | 6.3 | 0 | 2 | 85 | 2 | 56 | Milochondial inner membrane | Transporter activity: Catalytic activity |
| | | milochondial precursor | | | | | | | | | |
| 13 | IPI00645708 | Lamin B receptor variant | 70651.06 | 9.41 | 8 | 3 | 89 | 3 | 67 | Nuclear inner membrane | binding |
| 14 | IPI00328415 | HADH-cytochrome b5 reduclase | 34081.68 | 7.31 | 0 | 3 | 86 | 2 | 76 | ER Membrane: milochondial | Catalytic activity: Transporter |
| | | | | | | | | | | outer membrane | |
| 15 | IPI00654481 | Solute carrier family 3 (activator S of dibaSic and | 71076.20 | 4.84 | 1 | 2 | 85 | 4 | 64 | Plasma membrane | Transporter activity |
| | | neutral amino acid traSport), [symbol] | | | | | | | | | |
| 16 | IPI00465442 | ATP-binding cassette half-tansporter | 99648.17 | 9.26 | 9 | 4 | 52 | 2 | 59 | Plasma membrane | Catalytic activity binding |
| 17 | IPI00556382 | Equillibrative nucleoside transporter 1 | 58824.58 | 8.49 | 11 | 2 | 79 | 2 | 52 | Plasma membrane | Transporter activity |
| 18 | IPI00219728 | Milochondial 2-oxo [symbol] carrier protein | 33908.81 | 8.92 | 0 | 2 | 84 | 1 | 61 | Milochondial Membrane | Binding: Transporter activity |
| 19 | IPI00028264 | Cytochrome c1 [symbol] protein, milochondial precursor | 35367.00 | 9.15 | 0 | 2 | 110 | 1 | 75 | Milochondial Membrane | Transporter activity |
| 20 | IPI00237806 | Splice Isoform 3 of Spectrin beta chain erythrocyle | 242580.00 | 5.2 | 0 | | | 5 | 83 | Plasma membrane | Structural nolecular activity |
| 21 | IPI00027769 | [symbol] precursor | 28499.79 | 9.71 | 1 | 1 | 123 | 1 | 101 | Plasma membrane (Extracellular) | Catalytic activity |
| 22 | IPI00021766 | Splice Isoform 1 of Relicolon 4 | 40292.96 | 4.71 | 2 | 1 | 117 | 1 | 52 | ER membrane protein | Binding: Signal transducer apoptosis |
| 23 | IPI00220416 | Ubiquinol-cytochrome c reduclase complex 12 kDa | 13390.94 | 8.75 | 0 | 2 | 59 | 3 | 81 | Milochondial inner membrane | Catalytic activity: Transporter |
| | | protein | | | | | | | | | |
| 24 | IPI00027180 | CaaX Prenyl protease 1 homolog | 54777.53 | 7.12 | 8 | 1 | 97 | 1 | 93 | ER membrane: Golgo Plasma | Catalytic activity |
| | | | | | | | | | | membrane | |
| 25 | IPI00005202 | Membrane associated progesterone receptor | 23803.73 | 4.76 | 1 | 1 | 94 | 1 | 44 | Microsome membrane: plasmam | Signal transducer (receptor activity): |
| | | component 2 | | | | | | | | membrane | binding |
| 26 | IPI00646848 | Growth- [symbol] protein 12 | 78334.00 | 8.54 | 0 | 1 | 91 | 1 | 64 | Plasma membrane (Extacellular) | Catalytic activity: binding |
| 27 | IPI00396887 | [symbol] domain containing protein 1 precursor | 31770.80 | 4.92 | 3 | 1 | 85 | 1 | 69 | Plasma membrane: ER membrane | Tranporter activity |
| 28 | IPI00027448 | ATP synthase beta chain milochondial | 58524.60 | 5.28 | 0 | 1 | 82 | 1 | 49 | Milochondial inner membrane | Binding: Catalytic activity |
| 29 | IPI00024742 | Ubiquinol-cytochrome c reductase complex | 9769.08 | 10.08 | 0 | 1 | 82 | 1 | 42 | Milochondial inner membrane | Transporter: Catalytic activity |
| | | ubiquinone-binding protein OP-C | | | | | | | | | |
| 30 | IPI00013847 | Ubiquinol-cytochrome c reductase complex core | 52585.42 | 5.94 | 0 | 2 | 48 | 4 | 58 | Milochondial inner membrane | Catalytic activity: Transporter |
| | | protein 1 milochondial precursor | | | | | | | | | |
| 31 | IPI00028064 | Cathepsin G precursor | 28819.07 | 11.19 | 0 | 1 | 79 | 1 | 45 | Plasma membrane (associated): | Catalytic activity |
| | | | | | | | | | | intermediate | |
| 32 | IPI00100747 | Ubiquinol-cytochrome c reductase complex -like | 38927.58 | 4.31 | 1 | 1 | 70 | 1 | 90 | Plasma membrane (associated) | Transporter activity |
| | | protein KIAA1162 precursor | | | | | | | | | |
| 33 | IPI00465318 | Cytochrome c | 11610.09 | 9.53 | 0 | 1 | 62 | 1 | 38 | Milochondial inner membrane | Transporter activity |
| 34 | IPI00220459 | V blood group glycoprotein | 82770.92 | 8.08 | 1 | 1 | 53 | 2 | 93 | Plasma membrane | Catalytic activity: binding |
| 35 | IPI00552514 | Splice Isoform 1 at Vacoular protein translocating | 86350.11 | 6.02 | 7 | 1 | 47 | 1 | 54 | Vesicle membrane: Plasma | Catalytic activity: Transporter |
| | | ATPase 116 kDa subunit a isoform 1 | | | | | | | | membrane | |
| 36 | IPI00168091 | Olfactory receptor I IH4 | 36861.19 | 9.05 | 7 | 1 | 47 | 1 | 39 | Plasma membrane | Receptor activity |
| 37 | IPI00301100 | CDNA PSEC0252 fis, clone NT2RP3003250 highly | 54494.25 | 8.71 | 11 | 1 | 47 | 1 | 54 | ER membrane protein: Plasma | Unclassified |
| | | similar to [symbol] ortholog of mouse [symbol] | | | | | | | | membrane | |
| 38 | IPI00002372 | ATP-binding casstter sub-family D member 3 | 75427.57 | 8.41 | 3 | 1 | 44 | 2 | 64 | Perbisome membrane | Catalytic activity: binding |
| 39 | IPI00007426 | PRA1 family protein 3 | 21500.41 | 9.77 | 3 | 1 | 43 | 1 | 49 | ER membrane | Binding |
| 40 | IPI00026111 | Membrane protein | 21161.16 | 9.77 | 2 | 1 | 110 | 1 | 59 | ER and Golgi appartus membrane | Unclassified |
| 41 | IPI00022275 | Suppressarul [symbol] 1 | 66908.01 | 6.88 | 2 | 1 | 45 | | | ER membrane: Golgi Plasma | Catalytic: Binding |
| | | | | | | | | | | membrane | |
| 42 | IPI00100810 | NSPC051 | 14391.31 | 10.96 | 0 | 1 | 41 | | | Milochondial Membrane | Catalytic |
| 43 | IPI00007168 | ASP/ATP translocase 2 | 32743.13 | 9.76 | 2 | 7 | 101 | | | Milochondial inner membrane | Binding: Transporter activity |
| 44 | IPI00015826 | ATP-binding cassette sub-family B member 10, [symbol] | 79048.95 | 9.91 | 5 | 5 | 72 | | | Milochondial inner membrane | Binding: Catalytic activity |
| | | precursor | | | | | | | | | |
| 45 | IPI00027252 | B-cell receptor-associated protein 6AP37 | 33276.92 | 9.63 | 0 | 5 | 50 | | | Milochondial Membrane | Signal transducer: receptor (binding) |
| 46 | IPI00412713 | SAM50-like protein CGI-51 | 51829.30 | 6.44 | 0 | 3 | 93 | | | Milochondial inner membrane | Transporter: Catalytic activity: binding |
| 47 | IPI00025874 | [symbol] -protein [symbol] 67 kDa subunit precursor | 68526.81 | 5.98 | 1 | 3 | 93 | | | ER membrane | Catalytic activity: binding |
| 48 | IPI00470829 | Splice Isoform 3 of Milochondial inner membrane | 79977.51 | 6.31 | 0 | 3 | 59 | | | Milochondial inner membrane | Structural molecular activity: binding: |
| | | protein (Prolification-inducing gene 4 [symbol] | | | | | | | | | motor: apoptosis |
| 49 | IPI00294779 | Splice Isoform 2 of Voltage-cependent | 30770.33 | 8.85 | 0 | 2 | 100 | | | Milochondial outer membrane | Transporter activity |
| | | anon-selective channel protein 3 | | | | | | | | | |
| 50 | IPI00440493 | ATP synthase alpha chain milochondial precursor | 59712.99 | 9.15 | 0 | 3 | 69 | | | Milochondial Membrane | Transporter activity: binding: catalytic |
| | | | | | | | | | | | activity |
| 51 | IPI00383240 | Mitochondial substrate carrier [symbol] protein | 29874.04 | 9.68 | 0 | 3 | 59 | | | Milochondial inner membrane | Binding |
| 52 | IPI00339385 | Splice Isoform 2 of Retinol dehydrogenase 11 | 33381.68 | 8.95 | 0 | 2 | 101 | | | ER membrane | Catalytic activity |
| 53 | IPI00641334 | Similar to Cylochrome b5 outer milochondial | 14163.00 | 5.06 | 0 | 2 | 86 | | | Milochondial outer membrane | Unclassified |
| | | membrane isoform precursor | | | | | | | | | |
| 54 | IPI00096986 | Splice Isoform A of protein C25orf108 | 20411.22 | 10.45 | 3 | 2 | 89 | | | ER membrane: milochondial: | Transporter activity: Catalytic activity |
| | | | | | | | | | | microsome | |
| 55 | IPI00009345 | Transmembrane protein 14C | 11568.97 | 6.57 | 4 | 2 | 53 | | | ER membrane: Golgi membrane | unknown |
| 56 | IPI00039810 | Tricarboxytate transport protein milochondial | 35972.12 | 10.12 | 0 | 3 | 60 | | | Milochondial inner membrane | Binding: Transporter activity |
| | | precursor | | | | | | | | | |
| 57 | IPI00025796 | NADH ubiquinone oxidoreductase 30 kDa subunit | 30222.71 | 6.99 | 0 | 2 | 74 | | | Milochondial inner membrane | Catalytic activity: transporter activity: |
| | | milochondial precursor | | | | | | | | | binding |
| 58 | IPI00485155 | Rhesus blood group. CcEe antigens isoform 1 | 45421.20 | 9.4 | 12 | 2 | 61 | | | Plasma membrane | Transporter activity |
| 59 | IPI00003833 | Milochondial carrier homolog 2 | 33308.86 | 6.25 | 0 | 2 | 55 | | | Milochondial inner membrane | Binding |
| 60 | IPI00395769 | Splice Isoform Heart of ATP synthase gamma chain | 32860.24 | 9.31 | 0 | 2 | 55 | | | Milochondial inner membrane | Transporter activity: Catalytic activity |
| | | milochondial precursor | | | | | | | | | |
| 61 | IPI00542218 | 17 kDa protein | 16710.62 | 6.49 | 3 | 1 | 92 | | | ER membrane | Transporter activity: Catalytic activity: |
| | | | | | | | | | | | signal transducer activity (receptor) |
| 62 | IPI00293073 | Milochondial transmembrane GTPase FZO-2 | 66938.53 | 5.99 | 0 | 2 | 61 | | | Milochondial Membrane | Binding: catalytic activity |
| 63 | IPI00017510 | Cytochrome c oxidase subunit 2 | 25548.21 | 4.67 | 2 | 1 | 80 | | | Milochondial inner membrane | Transporter activity: binding: catalytic |
| | | | | | | | | | | | activity |
| 64 | IPI00027409 | [symbol] precursor | 27786.27 | 6.72 | 0 | 2 | 52 | | | PLasma membrane (Extracellular) | Ctalytic activity: binding: signal |
| | | | | | | | | | | cytoplasmic | transduce |
| 65 | IPI00644458 | SM-11044 binding protein | 29918.72 | 5.52 | 1 | 2 | 48 | | | Endosomal membrane | Transporter |
| 66 | IPI00216208 | Voltage-dependent anion-selective channel | 30622.53 | 6.63 | 0 | 2 | 50 | | | Milochondial outer membrane: | transporter activity |
| | | protein 1 | | | | | | | | Plasma [symbol] | |
| 67 | IPI00016342 | Ras-related protein Rab-7 | 23474.84 | 6.4 | 0 | 1 | 79 | | | [symbol] : Golgi membrane | Catalytic activity: Transporter: binding |
| 68 | IPI00452747 | KIAA0102 protein | 14159.28 | 7.65 | 1 | 2 | 38 | | | ER membrane: Microsome membrane | Catalytic activity |
| 69 | IPI00022246 | [symbol] precursor | 26668.65 | 9.75 | 0 | 1 | 75 | | | Plasma membrane (Extracellular) | Catalytic activity: binding: signal |
| | | | | | | | | | | cytoplasmic | transducer |
| 70 | IPI00009167 | Sodium/potassium-transporting ATPase beta-3 | 31492.09 | 8.58 | 1 | 1 | 71 | | | Plasma membrane | Transporter adhesion: catalytic activity |
| | | chain | | | | | | | | | |
| 71 | IPI00215610 | 55 kDa erythrocyte membrane protein | 62263.65 | 6.91 | 0 | 1 | 70 | | | Plasma membrane | catalytic activity (binding) |
| 72 | IPI00217169 | Splice Isoform XB of Plasma membrane calcium- | 133845.70 | 6.04 | 8 | 2 | 41 | | | Plasma membrane | Binding: catalytic activity transporter |
| | | transporting ATPase 4 | | | | | | | | | |
| 73 | IPI00023610 | Ras-related protein Rab-5A | 23543.82 | 6.32 | 0 | 1 | 69 | | | Endosome: Goldi membrane | Catalytic activity: Transporter: binding |
| 74 | IPI00019472 | Neutral amino acid transporter B | 58578.27 | 5.34 | 9 | 1 | 68 | | | Plasma membrane | Signal transducer (receptor [symbol] ): |
| | | | | | | | | | | | transporter |
| 75 | IPI00382815 | Splice Isoform 2 of Protein GPR107 precursor | 61936.41 | 6.72 | 7 | 1 | 68 | | | Plasma membrane | Signal transducer: catalytic: binding |
| 76 | IPI00007067 | Goldi-associated [symbol] -related protein 1 | 17076.48 | 9.44 | 0 | 1 | 66 | | | Golgi membrane: Extracellular | Unclassified |
| 77 | IPI00016513 | Ras-related protein Rab-10 | 22528.59 | 8.59 | 0 | 1 | 65 | | | Endosome: Golgi membrane | Catalytic activity: Transporter: binding |
| 78 | IPI00549893 | ATP synthase F0 subunit 6 | 24735.00 | 10.09 | 6 | 1 | 65 | | | Milochondial inner membrane | Transporter activity |
| 79 | IPI00010746 | [symbol] synthase 1 | 56491.10 | 8.71 | 9 | 2 | 33 | | | Milochondial Membrane | Catalytic activity |
| 80 | IPI00335277 | Splice Isoform 2 of [symbol] -like profellin | 26394.49 | 6.78 | 3 | 1 | 64 | | | Vesicle membrane: Plasma membrane | Transporter activity: Binding |
| 81 | IPI00007755 | Ras-related protein Rab-21 | 24201.21 | 6.18 | 0 | 1 | 62 | | | Endosome: Golgi membrane | Catalytic activity: Transporter: binding |
| 82 | IPI00447608 | S4 C27A2 protein | 64574.28 | 8.73 | 1 | 1 | 61 | | | ER membrane: [symbol] membrane | catalytic activity |
| 83 | IPI00032831 | Synoplosomal-associated protein 29 | 28852.59 | 5.56 | 0 | 1 | 58 | | | Plasma membrane: [symbol] | Transporter |
| 84 | IPI00385258 | Milochondial carrier homolog 1 soform b | 41517.29 | 9.4 | 2 | 1 | 57 | | | Milochondial membrane: intracellular | binding |
| 85 | IPI00221298 | Splice Isoform 2 of NADH ubiquinon oxido- | 59836.33 | 8.51 | 0 | 1 | 56 | | | Milochondial inner membrane | catalytic activity: transporter |
| | | reductase 51 kDa subunit milochondial precursor | | | | | | | | | |
| 86 | IPI00017334 | Prohibilin | 29785.90 | 5.57 | 0 | 1 | 54 | | | Milochondial Membrane | signal transducer: binding |
| 87 | IPI00429490 | Aquaporin 1 [symbol] 2 | 1491358 | 5.48 | 1 | 1 | 53 | | | Plasma membrane | transporter activity: Structural |
| | | | | | | | | | | | molecular activity |
| 88 | IPI00034208 | BCG induced integral membrane protein [symbol] 103 | 49508.38 | 5.71 | 7 | 1 | 53 | | | Plasma membrane | catalytic activity: transporter activity |
| 89 | IPI00644824 | Similar to [symbol] -associated protein delta subunit | 13210.66 | 6.49 | 1 | 1 | 51 | | | ER membrane | Binding |
| | | precursor | | | | | | | | | |
| 90 | IPI00419898 | ATPase H+ transporting lycosomal V0 subunit | 98018.34 | 6.18 | 6 | 1 | 49 | | | Vacuolar membrane: lysosome | transporter activity: catalytic |
| | | a isoform 2 | | | | | | | | | |
| 91 | IPI00320815 | Ubiquin-specific protease 31 | 118956.15 | 5.75 | 1 | 1 | 48 | | | Nuclear membrane | catalytic activity |
| 92 | IPI00640745 | Der1-like domain containing protein | 21238.77 | 8.09 | 4 | 1 | 48 | | | ER membrane | Unclassified |
| 93 | IPI00215848 | ATP Synthase e chain nilochondial | 7797.28 | 8.34 | 1 | 1 | 47 | | | Milochondial membrane | transporter activity |
| 94 | IPI00219802 | Erythrocyte band 7integral membrane protein | 31579.70 | 7.8 | 1 | 1 | 47 | | | Plasma membrane | binding |
| 95 | IPI00221002 | Peroxisomal membrane protein 2 | 22108.98 | 10.68 | 4 | 1 | 46 | | | [symbol] membrane | unknown |
| 96 | IPI00619900 | AdAptor-relAted protein complex 2, mu 1 subunit | 19357.91 | 9.57 | 0 | 1 | 46 | | | | |
| | | isoform b | | | | | | | | (ass Plasma membrane): [symbol] | |
| 97 | IPI00007084 | Milochondial aspartate-glutamate carrier protein | 74256.70 | 8.79 | 0 | 1 | 45 | | | Milochondial inner membrane: | transporter: binding |
| | | | | | | | | | | Plasma [symbol] | |
| 98 | IPI00221391 | NADH dehydrogenase subunit 5 | 66988.42 | 9.14 | 15 | 1 | 45 | | | Milochondial inner membrane | Transporter: catalytic activity |
| 99 | IPI00218128 | Splice Isoform Glycophorin D of Glycophorin C | 11491.61 | 4.76 | 1 | 1 | 44 | | | Plasma membrane | cell adhesion: signal transducer |
| | | | | | | | | | | | (receptor) |
| 100 | IPI00549891 | Heparan sulfate 2-Osulfotransfase 1 | 41844.40 | 8.83 | 0 | 1 | 43 | | | Nucleear membrane | catalytic activity |
| 101 | IPI00376529 | 8 kDa protein | 9459.86 | 6.75 | 1 | 1 | 43 | | | Milochondial membrane | catalytic: transporter |
| 102 | IPI00290928 | Gianine nucleotide-binding protein alpha-13 | 44021.66 | 8.11 | 0 | 1 | 42 | | | Plasma membrane | Signal transducer (receptor): binding: |
| | | subunit | | | | | | | | | catalytic activity |
| 103 | IPI00009247 | Full lenght cDNA clone CS0DC006YH13 of | 8609.50 | 9.94 | 1 | 1 | 41 | | | Milochondial membrane | Unclassified |
| | | Neuroblastoma of Homo sapiens | | | | | | | | | |
| 104 | IPI00396411 | [symbol] and palate transmembrane protein 1 | 76048.46 | 5.88 | 5 | 1 | 40 | | | Plasma membrane | Unclassified |
| 105 | IPI00607576 | Splice isoform I of protein C9orf5 | 100881.01 | 9.03 | 14 | 1 | 40 | | | Plasma membrane | unknown |
| 106 | IPI00007676 | Steroid dehydrogenase homolog | 34328.24 | 9.34 | 3 | 1 | 87 | | | ER membrane multipass memb | catalytic activity |
| 107 | IPI00642244 | Novel protein | 109351.93 | 7.94 | 0 | 1 | 40 | | | ER membrane | Unclassified |
| 108 | IPI00180121 | Splice Isoform A of Chloride channel protein 6 | 35910.30 | 8.03 | 3 | 1 | 41 | | | Plasma membrane | ion channel activity |
| 109 | IPI00184474 | Splice Isoform 3 of Protein GPR107 precursor | 61936.41 | 6.72 | 7 | 1 | 68 | | | Plasma membrane | Signal tranducer catalytic: binding |
| 110 | IPI00643646 | NADH dehydrogenase | 52526.70 | 7.21 | 0 | 1 | 69 | | | Milochondial inner membrane | Tranporter: catalytic |
| 111 | IPI00464963 | Hemogen | 55278.74 | 4.82 | 0 | | | 4 | 101 | Golgi membrane: Visicle Nuclear | Structural molecular activity |
| 112 | IPI00220468 | ATP synthase H+ transporting milochindial F0 | 18479.50 | 5.21 | 0 | | | 3 | 90 | Michondial inner membrane | Structural molecular activity: |
| | | complex subunit d isoform a | | | | | | | | | catalytic activity transporter activity |
| 113 | IPI00552913 | Splice Isoform 2 of Syntaxin-7 | 27383.66 | 5.02 | 0 | | | 2 | 66 | Endosome membrane | Transporter activity |
| 114 | IPI00176681 | OTTHUMO00000016310 | 12179.15 | 9.3 | 0 | | | 2 | 76 | Milochondial membrane | Transporter activity: Catalytic acivity |
| 115 | IPI00216085 | Cytochrome c oxidase polypepide Vib | 10054.68 | 6.78 | 0 | | | 2 | 75 | Milochondial membrane | Transporter activity: Catalytic acivity |
| 116 | IPI00298268 | Splice Isoform Short of Prostagladin G/H | 64440.72 | 7.9 | 0 | | | 2 | 39 | Microsome Membrane: Cytoplasmic | Catalytic activity: Antioxidant |
| | | synthase 1 precursor | | | | | | | | | |
| 117 | IPI00005695 | NADH-ubiquinone oxidoreductase MWFE subunit | 8067.13 | 8.93 | 1 | | | 1 | 64 | Milochondial Membrane | Catalytic activity: Transporter |
| 118 | IPI00018311 | Stromal cell-derived receptor-1 beta | 44359.50 | 8.11 | 1 | | | 1 | 59 | Plasma membrane | Receptor activity |
| 119 | IPI00020510 | Protein C10orf70 | 12191.20 | 9.19 | 1 | | | 1 | 56 | Michondial membrane | motor: Binding |
| 120 | IPI00292532 | Antibacterial protein FALL-39 precursor | 19578.27 | 9.48 | 1 | | | 1 | 55 | Plasma membrane | Catalytic (Enzyme inhibitor activity) |
| 121 | IPI00043429 | Hypothetical Protein FLJ31842 | 30020.92 | 9.4 | 6 | | | 1 | 54 | Plsama membrane | Unclassified |
| 122 | IPI00255052 | NADH-ubiquinone oxidireductase B22 subunit | 21665.80 | 8.59 | 0 | | | 1 | 54 | Milochondial Membrane | Ctalytic activity, Transporter |
| 123 | IPI00010845 | NADH-ubiquinone oxidireductase 23 kDa subunit, | 23689.62 | 6 | 0 | | | 1 | 51 | Milochondial Membrane | Catalytic activity: Tranporter |
| | | milochondial precursor | | | | | | | | | |
| 124 | IPI00553138 | Vesicle-associated membrane protein 2 | 12509.63 | 7.82 | 1 | | | 1 | 50 | Vesicle membrane: plasma | Transporter activity |
| | | | | | | | | | | membrane protein | |
| 125 | IPI00032230 | Splice Isoform A of Band 4.1-like protein 3 | 120603.14 | 5.09 | 0 | | | 1 | 48 | Plasma membrane protein | Structural molecular activity: binding |
| | | | | | | | | | | cytoskeleton | |
| 126 | IPI00641145 | ATPase subunit 8 | 7972.15 | 9.93 | 1 | | | 1 | 47 | Milochondial membrane protein | Tranporter activity |
| 127 | IPI0031804 | Splice Isoform 1 of Voltage-dependent | 30639.28 | 8.85 | 0 | | | 1 | 46 | Milochondial puter membrane | Transporter |
| | | anion-selective channel protein 3 | | | | | | | | | |
| 128 | IPI00292290 | Splice Isoform Long of Demalin | 45486.22 | 8.94 | 0 | | | 1 | 45 | Plasma membrane: cytoskeleton | Binding (actin) |
| 129 | IPI00549805 | ATP synthase H+ transporting milochondial | 59771.60 | 9.07 | 0 | | | 1 | 42 | Milochondial inner membrane | Transporter: Binding Catalytic |
| | | F1 complex alpha subunit isoform a | | | | | | | | | activity |
| 130 | IPI00218466 | Sec861 alpha 1 subunit | 52230.51 | 8.3 | 10 | | | 1 | 41 | ER membrane | Transporter |
| 131 | IPI00003950 | Vesicle integral-membrane protein ViP35 precursor | 40203.10 | 6.46 | 1 | | | 1 | 41 | ER membrane: Golgi: Plasma | Tranporter activity: Binding |
| | | | | | | | | | | membrane | |
| 132 | IPI00219685 | Cell death-regulatory protein GRIM19 | 25804.38 | 9.82 | 1 | | | 1 | 40 | Milochondial inner membrane | Catalytic: Tranporter: Binding: |
| | | | | | | | | | | | Apoplosis |
| 133 | IPI00555919 | NDUFC2 protein | 14164.40 | 9.04 | 1 | | | 1 | 35 | Milochondial inner membrane | Catalytic: Transporter |

[symbol]

indicates data missing or illegible when filed

TABLE 4
Non-membrane proteins of FPNRBCs

| | | | | | | TFE | | MeOH | | | |
|---|---|---|---|---|---|---|---|---|---|---|---|
| | | | Protein | Protein | TMHMM | Peptide | Best Ion | Peptide | Best Ion | | |
| | Accessions | Protein description | MW | PI | (V2) | Count | Score | Count | Score | Location | Molecular function |
| 1 | IPI00453473 | Histone H4 | 11229.34 | 11.3 | 0 | 12 | 122 | 11 | 116 | Nuclear | Binding |
| 2 | IPI00217471 | Hemoglobin epsilon chain | 16061.43 | 8.86 | 0 | 5 | 164 | 6 | 170 | Cytoplasmic | Transporter activity |
| 3 | IPI00152785 | Histone H2B n | 13786.52 | 10.32 | 0 | 6 | 148 | 9 | 128 | Nuclear | Binding |
| 4 | IPI00375676 | Femilin light chain | 28399.25 | 6 | 0 | 4 | 134 | 3 | 104 | Cytoplasmic (Femilin complex) | Binding (Iron) |
| 5 | IPI00218448 | Histone H2A z | 13413.51 | 10.58 | 0 | 4 | 87 | 4 | 83 | Nuclear | Binding |
| 6 | IPI00219038 | H3 histone, family 3B | 15316.50 | 11.27 | 0 | 5 | 85 | 6 | 147 | Nuclear | Binding |
| 7 | IPI00646240 | Hypothetical protein | 7390.90 | 9.66 | 0 | 3 | 112 | 4 | 88 | Nuclear | Binding |
| 8 | IPI00236554 | Splice Isoform H14 of Myeloperoxidase | 73806.61 | 9.3 | 0 | 4 | 79 | 8 | 77 | Lysosome: Nuclear | Binding: Catalytic: Transporter: |
| | | precursor | | | | | | | | | Antioxidant |
| 9 | IPI00013415 | 40S ribosomal protein 57 | 22113.26 | 10.09 | 0 | 3 | 100 | 6 | 89 | Ribosomal | Structural molecular activity |
| 10 | IPI00647085 | Ribosomal protein L5 variant | 34340.69 | 9.73 | 0 | 3 | 131 | 5 | 72 | Ribosomal | Structural molecular activity |
| 11 | IPI00025039 | Fibrillarin | 33763.42 | 10.18 | 0 | 3 | 94 | 6 | 72 | Nuclear | Binding (RNA) |
| 12 | IPI00003968 | NADH-ubiquinon oxidoreductase 39 kDa | 42482.57 | 9.81 | 0 | 3 | 100 | 2 | 91 | Milochondial | Catalytic activity: Transporter |
| | | subunit milochondial precursor | | | | | | | | | |
| 13 | IPI00176628 | PREDICTED: similar to ribosomal protein L18a | 20753.89 | 10.73 | 0 | 4 | 71 | 4 | 49 | Ribosomal | Structural molecular activity |
| 14 | IPI00027270 | 60S ribosomal protein L26 | 17247.53 | 10.55 | 0 | 3 | 84 | 1 | 44 | Ribosomal | Structural molecular activity |
| 15 | IPI00454095 | H2B/t variant | 21458.16 | 10.71 | 0 | 3 | 84 | 4 | 83 | Nuclear | Binding |
| 16 | IPI00093057 | Coproporphyrinogen III oxidase milochondial | 50119.97 | 8.59 | 0 | 4 | 56 | 4 | 59 | Milochondial | Catalytic activity |
| | | precursor | | | | | | | | | |
| 17 | IPI00386491 | Splice Isoform Short of Heterogenous nuclear | 88890.18 | 5.8 | 0 | 3 | 88 | 6 | 123 | Nuclear | Binding |
| | | ribonucleoprotein U | | | | | | | | | |
| 18 | IPI00552125 | HNRPC protein | 27804.46 | 4.55 | 0 | 2 | 80 | 1 | 59 | Nuclear | Binding |
| 19 | IPI00411937 | Nuclear protein Ncp56 | 66194.76 | 9.21 | 0 | 2 | 66 | 3 | 61 | Nuclear | Chaperone: binding |
| 20 | IPI00219155 | 60S ribosomal protein L27 | 15656.71 | 10.56 | 0 | 2 | 68 | 3 | 63 | Ribosomal | Structural molecular activity |
| 21 | IPI00455758 | Ribosomal protein L27a | 16468.03 | 11 | 0 | 2 | 76 | 2 | 93 | Ribosomal | Structural molecular activity |
| 22 | IPI00550021 | 60S ribosomal protein L3 | 45948.72 | 10.19 | 0 | 2 | 73 | 6 | 96 | Ribosomal | Structural molecular activity |
| 23 | IPI00217030 | 40S ribosomal protein S4, X isoform | 29448.01 | 10.16 | 0 | 2 | 73 | 2 | 56 | Ribosomal | Structural molecular activity |
| 24 | IPI00148006 | H2A histane family member Y isoform 1 | 39159.16 | 9.53 | 0 | 1 | 103 | 3 | 97 | Nuclear | Binding |
| 25 | IPI00037070 | Splice Isoform 2 of Heat shock cognate 71 | 53887.70 | 5.74 | 0 | 2 | 61 | 2 | 46 | Cytoplasmic: nuclear | Chaperone: binding |
| | | kDa protein | | | | | | | | | |
| 26 | IPI00514123 | 18 kDa protein | 17639.75 | 10.86 | 0 | 2 | 61 | 3 | 58 | Ribosomal | Structural activity: binding |
| 27 | IPI00219486 | Splice Isoform 2 of 40S ribosomal protein S24 | 15059.24 | 10.89 | 0 | 1 | 91 | 1 | 79 | Ribosomal | Structural molecular activity |
| 28 | IPI00182533 | 60S ribosomal protein L28 | 15606.63 | 12.02 | 0 | 1 | 91 | 1 | 74 | Ribosomal | Structural molecular activity |
| 29 | IPI00646415 | RAB14 member RAS oncogene family | 20396.31 | 5.94 | 0 | 1 | 78 | 1 | 38 | Universal | Binding: Catalytic |
| 30 | IPI00006000 | [symbol] peroxicase precursor | 80989.13 | 10.31 | 0 | 2 | 38 | 2 | 41 | Lysosome: Nuclear | Binding: Catalytic: Transporter: |
| | | | | | | | | | | | Antioxidant |
| 31 | IPI00216613 | Splice Isoform Short of Splicing factor, | 72217.75 | 9.26 | 0 | 1 | 68 | 1 | 72 | Nuclear | Binding |
| | | proline-and glutamine-rich | | | | | | | | | |
| 32 | IPI00440703 | GSTK1 protein | 31545.51 | 8.85 | 0 | 1 | 61 | 1 | 75 | Milochondial | Catalytic: Binding |
| 33 | IPI00396485 | 50 kDa protein | 50136.50 | 7.25 | 0 | 1 | 69 | 1 | 54 | Nuclear: Cytoplasmic | Binding translation: catalytic |
| | | | | | | | | | | | activity |
| 34 | IPI00021924 | Histone H1x | 22473.53 | 10.76 | 0 | 1 | 52 | 1 | 79 | Nuclear | Binding |
| 35 | IPI00020021 | DEK protein | 42647.92 | 8.69 | 0 | 1 | 49 | 3 | 53 | Nuclear | Binding transcription regulation |
| | | | | | | | | | | | activity |
| 36 | IPI00639942 | Ribosomal pRotein S29 isofoRm 2 | 8081.99 | 10.08 | 0 | 1 | 39 | 1 | 39 | Ribosomal | Structural molecular activity |
| 37 | IPI00022832 | Brain Protein 44 | 11673.16 | 10.21 | 0 | 1 | 74 | 1 | 62 | Unclassified | Transporter activity: Binding |
| 38 | IPI00220855 | H2A histone family, member J. isoform 2 | 14010.93 | 10.9 | 0 | 7 | 132 | | | Nuclear | Binding |
| 39 | IPI00339274 | Histone H2A q | 13848.60 | 10.9 | 0 | 7 | 121 | | | Nuclear | Binding |
| 40 | IPI00549250 | HP1 BP74 | 61169.27 | 9.69 | 0 | 3 | 111 | | | Nuclear | Binding |
| 41 | IPI00216587 | 40S ribosomal protein 58 | 24059.12 | 10.32 | 0 | 4 | 88 | | | Ribosomal | Structural molecular activity |
| 42 | IPI00011654 | Tubulin beta-2 chain | 49638.97 | 4.78 | 0 | 3 | 95 | | | Cytoplasmic (cytoskeleton) | Signal transducer: Structural |
| | | | | | | | | | | | molecular activity: binding |
| | | | | | | | | | | | chaperone |
| 43 | IPI00031357 | Protoporphyrinogen oxidase | 50733.65 | 8.44 | 0 | 3 | 81 | | | Milochondial | Catalytic activity: Transporter |
| 44 | IPI00472119 | PREDICTED: similar to ribosomal protein S3a | 29951.83 | 9.78 | 0 | 3 | 64 | | | Ribosomal | Structural molecular activity |
| 45 | IPI00334432 | 16 kDa protein | 15532.11 | 8.78 | 0 | 3 | 144 | | | Cytoplasmic | Transporter activity |
| 46 | IPI00450746 | ATP synthase H+ transporting milochondial F0 | 17451.13 | 9.38 | 0 | 2 | 95 | | | Milochondial | Transporter activity |
| | | complex subunit b isoform 2 | | | | | | | | | |
| 47 | IPI00383296 | Heterogeneous nuclear ribonucleoprotein Misoform b | 73572.36 | 8.94 | 0 | 2 | 100 | | | Nuclear | Binding |
| 48 | IPI00555878 | Probable DNA dC>dU editing enzyme APOBEC 3C | 22810.96 | 7.52 | 0 | 2 | 79 | | | Unclassified | Catalytic activity: binding |
| 49 | IPI00478327 | OTTHUMP00000028841 | 22617.43 | 10.43 | 0 | 2 | 96 | | | Ribosomal | Structural molecular activity: |
| | | | | | | | | | | | binding |
| 50 | IPI00554589 | 60S ribosomal protein L10 | 48583.91 | 8.95 | 0 | 1 | 133 | | | Milochondial | catalytic activity: binding |
| 51 | IPI00554723 | Hypothetical protein FLJ35097 | 24429.82 | 10.11 | 0 | 2 | 65 | | | Ribosomal | Structural molecular activity: |
| | | | | | | | | | | | binding |
| 52 | IPI00398234 | 21 kDa protein | 20886.74 | 9.45 | 0 | 2 | 60 | | | Ribosomal | Structural molecular activity: |
| | | | | | | | | | | | binding |
| 53 | IPI00025091 | 40S ribosomal protein S11 | 18418.99 | 10.31 | 0 | 2 | 60 | | | Ribosomal | Structural molecular activity |
| 54 | IPI00166335 | 10 kDa protein | 9733.91 | 5.81 | 0 | 1 | 90 | | | Nuclear | Binding (DNA) |
| 55 | IPI00450049 | ATP synthase, H+ transporting milochondial F0 complex, | 15763.17 | 6.6 | 0 | 2 | 57 | | | Milochondial | Transporter activity |
| | | subunit d isoform b | | | | | | | | | |
| 56 | IPI00477513 | 15 kDa protein | 15095.99 | 7.9 | 0 | 4 | 134 | | | Cytoplasmic | Transporter activity |
| | | | | | | | | | | (hemoglobin complex) | |
| 57 | IPI00216115 | Splice Isoform GN-1S of Glycogenin-1 | 29070.36 | 4.73 | 0 | 1 | 73 | | | Cytoplasmic | Catalytic activity |
| 56 | IPI00107750 | Optic atrophy 1 isoform 5 | 113445.91 | 7.63 | 0 | 2 | 43 | | | Milochondial | Motor; binding; catalytic activity |
| 59 | IPI00221092 | 40A ribosomal protein S16 | 16304.00 | 10.21 | 0 | 1 | 75 | | | Ribosomal | Structural molecular activity |
| 60 | IPI00172656 | Protein expressed in T-cells and eosinophils in alopic | 52590.54 | 6.45 | 0 | 1 | 73 | | | Nuclear: Cytoplasmic | unknown |
| | | dermatits | | | | | | | | | |
| 61 | IPI00007928 | Pre-mRNA Processing splicing factor 8 | 273426.56 | 8.95 | 0 | 2 | 40 | | | Nuclear | Binding |
| 62 | IPI00411968 | Protein | 17775.75 | 9.13 | 0 | 1 | 64 | | | Nuclear | Chaperone: Binding |
| 63 | IPI00470526 | 60S ribosomal protein L15 | 24000.04 | 11.62 | 0 | 2 | 43 | | | Ribosomal | Structural molecular activity |
| 64 | IPI00644559 | 12 kDa protein | 12027.62 | 6.81 | 0 | 1 | 70 | | | Milochondial | Transporter: catalytic activity |
| 65 | IPI00455976 | PREDICTED: similar to hypothetical protein | 27850.92 | 10.36 | 0 | 1 | 69 | | | Ribosomal | Structural molecular activity |
| 66 | IPI00219097 | High mobility group protein 2 | 23887.68 | 7.77 | 0 | 1 | 67 | | | Nuclear | binding: transriptions enzyme |
| | | | | | | | | | | | regulatory activity |
| 67 | IPI00104080 | Thyroid hormone receptor-associated protein comlex | 106829.04 | 10.16 | 0 | 1 | 65 | | | Nuclear | Transcription: signal transducer |
| | | 150 kDa component | | | | | | | | | binding |
| 68 | IPI00376215 | Splice Isoform 2 of DNA-dependent protein kinase | 465202.00 | 6.81 | 0 | 1 | 64 | | | Nuclear | catalytic activity |
| | | catalytic subunit | | | | | | | | | |
| 69 | IPI00031691 | 60S ribosomal protein L9 | 21849.80 | 9.98 | 0 | 1 | 58 | | | Ribosomal | Structural molecular activity |
| 70 | IPI00374249 | 13 kDa protein | 12579.21 | 10.65 | 0 | 1 | 57 | | | Ribosomal | Structural molecular activity |
| 71 | IPI00383085 | FLJ00144 protein | 26437.22 | 5.75 | 0 | 1 | 54 | | | Unclassified | Unclassified |
| 72 | IPI00471915 | 46 kDa protein | 47538.39 | 11.07 | 0 | 2 | 52 | | | Ribosomal | Structural molecular activity; |
| | | | | | | | | | | | binding |
| 73 | IPI00171459 | Hypothetical protein FLJ90397 | 36944.10 | 6.66 | 0 | 1 | 52 | | | Unclassified | catalytic activity |
| 74 | IPI00021439 | Actin cytoplasmic 1 | 41709.73 | 5.29 | 0 | 1 | 52 | | | cytoplasmic | Motor: structural molecular activity |
| 75 | IPI00469044 | RDC2 protein | 56049.20 | 9.02 | 0 | 1 | 51 | | | Nuclear | catalytic activity: binding; enzyme |
| | | | | | | | | | | | regulatory activity |
| 76 | IPI00020599 | [symbol] precursor | 48111.62 | 4.29 | 0 | 1 | 52 | | | cytoplasmic | binding transcription |
| 77 | IPI00299000 | Proliferation-associated 2G4 38 kDa | 43785.20 | 6.13 | 0 | 1 | 50 | | | Nuclear | Catalytic: binding |
| 78 | IPI00329745 | 130 kDa leucine-rich protein | 158625.65 | 5.91 | 0 | 1 | 48 | | | Nuclear: Cytolasmic | catalytic: binding |
| 79 | IPI00397441 | PREDICTED: similar to dJ75306 2 (novel protein | 13749.95 | 9.36 | 0 | 1 | 47 | | | Ribosomal | Structural molecular activity: |
| | | similar to RPS17 (40S ribosomal protein | | | | | | | | | binding |
| 80 | IPI00011107 | Isocitrate dehydrogenase \|NADP\| milochondial precursor | 50878.65 | 6.68 | 0 | 1 | 44 | | | Milochondial | Catalytic activity |
| 81 | IPI00513768 | Novel protein | 52673.49 | 9.76 | 0 | 1 | 43 | | | Unclassified | binding |
| 82 | IPI00604532 | 13 kDa differentiation-associated protein | 17073.62 | 9.63 | 0 | 1 | 42 | | | Milochondial | catalytic transporter |
| 83 | IPI00028888 | Splice Isoform 1 of Heteroganeous nuclear | 38410.33 | 7.62 | 0 | 1 | 41 | | | Nuclear | binding |
| | | ribinucleoprotein O0 | | | | | | | | | |
| 84 | IPI00055287 | Hypothetical protein FLJ32630 | 65726.57 | 8.35 | 0 | 1 | 41 | | | Unclassified | Unclassification |
| 85 | IPI00014106 | Protein p65 | 68575.97 | 9.57 | 0 | 1 | 41 | | | Nuclear | binding: transcription signal |
| | | | | | | | | | | | transducer (receptor) |
| 86 | IPI00335130 | 46 kDa protein | 46265.42 | 7.26 | 0 | 1 | 41 | | | Unclassified | Structural molecular activity: |
| | | | | | | | | | | | Catalytic |
| 87 | IPI00217553 | Milochondial ribosomal protein L41 | 15372.99 | 9.58 | 0 | 1 | 40 | | | Ribosomal | structural activity |
| 88 | IPI00383231 | Kelch domain containing protein 1 | 45661.73 | 5.67 | 0 | 1 | 39 | | | Unclassified | binding: transcription |
| 89 | IPI00419579 | P450-like protein | 52399.01 | 5.84 | 0 | 1 | 39 | | | Cytoplasmic | binding: catalytic |
| 90 | IPI00216457 | Histone H2A o | 13965.65 | 10.9 | 0 | | | 7 | 125 | Nuclear | Binding |
| 91 | IPI00217465 | Histone H1.2 | 21220.71 | 10.94 | 0 | | | 6 | 11 | Nuclear | Binding |
| 92 | IPI00217467 | Histone H1.4 | 21720.93 | 11.03 | 0 | | | 5 | 113 | Nuclear | Binding |
| 93 | IPI00217468 | Histone H1.5 | 22435.43 | 10.91 | 0 | | | 5 | 112 | Nuclear | Binding |
| 94 | IPI00179330 | Ubiquitin and ribosomal protein S27a | 17893.44 | 9.65 | 0 | | | 5 | 159 | Ribosomal | Structural moleculara activity: |
| | | | | | | | | | | | Catalytic |
| 95 | IPI00217466 | Histone H1.3 | 22205.28 | 11.02 | 0 | | | 5 | 103 | Nuclear | Binding |
| 96 | IPI00031562 | Histone H2A | 14112.93 | 11.05 | 0 | | | 7 | 121 | Nuclear | Binding |
| 97 | IPI00303133 | Histone H2bi | 13752.50 | 10.32 | 0 | | | 9 | 128 | Nuclear | Binding |
| 98 | IPI00217469 | Histone H1.1 | 21697.83 | 10.99 | 0 | | | 4 | 112 | Nuclear | Binding |
| 99 | IPI00645201 | Ribosomal protein S8 | 21866.01 | 1037 | 0 | | | 4 | 104 | Ribosomal | Structural molecular activity |
| 100 | IPI00554676 | Hemoglobin gamma-2 chain | 15965.25 | 6.71 | 0 | | | 4 | 146 | Cytoplasmic | Transporter activity |
| 101 | IPI00410714 | Alpha 2 globin variant | 15270.94 | 6.72 | 0 | | | 5 | 106 | Cytoplasmic | Transporter activity |
| 102 | IPI00025329 | HNRPR protein | 23451.25 | 11.48 | 0 | | | 4 | 89 | Ribosomal | Structural molecular activity |
| 103 | IPI00644055 | Ribosomal protein L10 variant | 71170.40 | 8.23 | 0 | | | 4 | 73 | Nuclear | Binding (RNA) |
| 104 | IPI00641164 | Ribosomal protein L17 | 24526.87 | 10.11 | 0 | | | 5 | 67 | Ribosomal | Structural molecular activity |
| 105 | IPI00413324 | 60S ribosomal protein L17 | 21252.29 | 10.18 | 0 | | | 3 | 71 | Ribosomal | Structural molecular activity |
| 106 | IPI00402185 | OTTHUMO00000016816 | 62617.34 | 7.18 | 0 | | | 3 | 73 | Nuclear: Cytoplasmic | Binding |
| 107 | IPI00216695 | Splice Isoform 2 Bromodomain adjacent to zinc | 170340.36 | 8.7 | 0 | | | 4 | 55 | Nuclear | Binding: Transcription activity |
| | | linger domain protein 1B | | | | | | | | | |
| 108 | IPI00455457 | Novel protein similar to histone 2.H3c | 15420.55 | 11.27 | 0 | | | 3 | 47 | Nuclear | Binding |
| 109 | IPI00455900 | PREDICTED: similar to 60S ribosomal protein L32 | 52270.86 | 10.1 | 0 | | | 1 | 118 | Ribosomal | Structural molecular activity |
| 110 | IPI00412607 | 60S ribosomal protein L35 | 14411.52 | 11.04 | 0 | | | 1 | 108 | Ribosomal | Structural molecular activity |
| 111 | IPI00301323 | ATP-dependent RNA helicase DDX18 | 75359.44 | 9.52 | 0 | | | 2 | 80 | Nuclear | |
| 112 | IPI00374234 | PREDICTED: similar to 60S ribosomal protein L21 | 10973.01 | 10.97 | 0 | | | 2 | 51 | Ribosomal | Structural molecular activity |
| 113 | IPI00297211 | SWI/SNF related matrix associated actin dependent | 121828.36 | 6.27 | 0 | | | 1 | 81 | Nuclear | Catalytic activity: binding: |
| | | regulator of chomatin subfamily A me | | | | | | | | | transcription activity |
| 114 | IPI00382950 | Beta-globin gene from a thalassomia patient complete cds | 18918.59 | 6.28 | 0 | | | 2 | 50 | Cytoplasmic | Transporter activity |
| 115 | IPI00473011 | Hemoglobin delta chain | 15914.25 | 7.97 | 0 | | | 2 | 50 | Cytoplasmic | Transporter activity |
| 116 | IPI00065554 | Hypothetical protein FLJ32119 | 62438.95 | 9.43 | 0 | | | 1 | 73 | Nuclear: Cytoplasmic | Binding: Transcription |
| 117 | IPI00607820 | Splice Isoform 2 of H/ACA ribonucleoprotein complex | 20821.40 | 10.45 | 0 | | | 1 | 71 | Nuclear | Transporter activity: Binding |
| | | subunit 1 | | | | | | | | | |
| 118 | IPI00030179 | 60S ribosomal L7 | 29207.20 | 10.66 | 0 | | | 2 | 102 | Ribosomal | Structural molecular activity |
| 119 | IPI00219153 | 60S ribosomal protein L22 | 14646.76 | 9.22 | 0 | | | 1 | 70 | Ribosomal | Structural molecular activity |
| 120 | IPI00549664 | HEAT repeat containing protein | 105626.65 | 9.45 | 0 | | | 1 | 69 | Nuclear | Catalytic activity |
| 121 | IPI00375511 | Ribosomal pRotein L15: 60S Ribosomal pRotein L15 | 24071.05 | 11.62 | 0 | | | 2 | 36 | Ribosomal | Structural molecular activity |
| 122 | IPI00397701 | PREDICTED: similar to 40S ribosomal protein S16 | 1640185 | 10.05 | 0 | | | 1 | 64 | Ribosomal | Structural molecular activity |
| 123 | IPI00604684 | NADH dehydrogenase | 30141.58 | 6.25 | 0 | | | 1 | 60 | Milochondial | Catalytic: Transporter: binding |
| 124 | IPI00021458 | EH-domain containing protein 3 | 61857.09 | 6.06 | 0 | | | 1 | 57 | Nuclear | Binding |
| 125 | IPI00479958 | Splice Isoform 2 of N-acylneuraminate cytidylyltransferase | 29543.15 | 8.59 | 0 | | | 1 | 52 | Unclassified | Catalytic activity |
| 126 | IPI00607584 | Splice Isoform 2 myb-binding protein 1A | 149273.88 | 9.34 | 0 | | | 1 | 49 | Nuclear: Cytoplasmic | Binding: Catalytic activity |
| 127 | IPI00478631 | 65 kDa protein | 64667.14 | 9.8 | 0 | | | 1 | 47 | Nuclear | Binding (DNA) |
| 128 | IPI00556310 | 58 kDa protein | 57968.46 | 10.11 | 0 | | | 1 | 47 | Ribosomal | Structural molecular activity |
| 129 | IPI0013452 | Hypothetical protein DKFZp313B047 | 170483.09 | 7.02 | 0 | | | 1 | 45 | Cytoplasmic | Catalytic activity: binding |
| 130 | IPI00166768 | TUBA6 protein | 36624.66 | 8.2 | 0 | | | 1 | 45 | Cytoplasmic (cytoskeleton) | Structural molecular: binding |
| 131 | IPI00215780 | 40S risobomal protein S19 | 15919.49 | 10.31 | 0 | | | 1 | 44 | Ribosomal | Structural molecular activity |
| 132 | IPI00386255 | Pol rotein | 97621.79 | 8.89 | 0 | | | 1 | 39 | Cytoplasmic | binding |

[symbol]

indicates data missing or illegible when filed

TABLE 5
Proteins of FPNRBCs with transmembare domain but location unknown

| | | | | | | TFE | | MeOH | | | |
|---|---|---|---|---|---|---|---|---|---|---|---|
| | | | | | | | Best | | Best | | |
| | | | Protein | Protein | TMHMM | Peptide | Ion | Peptide | Ion | | Molecular |
| | Accession # | Protein description | MW | PI | (V2) | Count | Score | Count | Score | Location | function |
| 1 | IPI00166079 | Vitamin K epoxide reductase | 19822.68 | 9.28 | 2 | 1 | 73 | | | unclassified | catalytic |
| | | complex subunit 1-like protein 1 | | | | | | | | | activity |
| 2 | IPI00604615 | ALEX3 protein variant | 42443.90 | 8.75 | 1 | 1 | 46 | | | unclassified | Unclassified |
| 3 | IPI00646289 | 25 kDa protein | 25141.15 | 8.93 | 1 | 4 | 113 | 3 | 77 | Unclassified | Unclassified |
| 4 | IPI00394779 | Splice Isoform 1 of Protein | 45068.18 | 8.87 | 1 | 1 | 40 | | | unclassified | catalytic |
| | | C20orf22 | | | | | | | | | activity |
| 5 | IPI00639803 | 8 kDa protein | 8078.35 | 9.01 | 2 | | | 1 | 45 | Unclassified | Unclassified |
| 6 | IPI00031064 | Hypothetical protein | 21513.48 | 9.36 | 2 | 1 | 61 | | | unclassified | Unclassified |
| | | DKFZp586C1924 | | | | | | | | | |
| 7 | IPI00176708 | Hypothetical protein | 6595.25 | 9.58 | 1 | 1 | 46 | | | unclassified | Unclassified |
| | | MGC14288 | | | | | | | | | |
| 8 | IPI00295621 | Hypothetical protein | 10137.19 | 9.52 | 2 | | | 1 | 100 | Integral | Unclassified |
| | | DKFZp564K247 | | | | | | | | membrane | |
| | | | | | | | | | | protein | |

Single Peptide Based Identification of Proteins

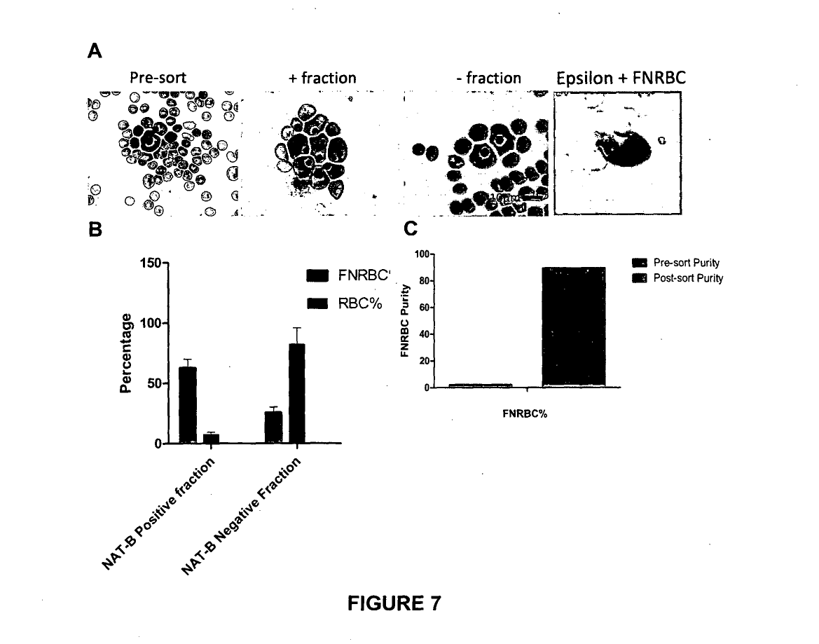

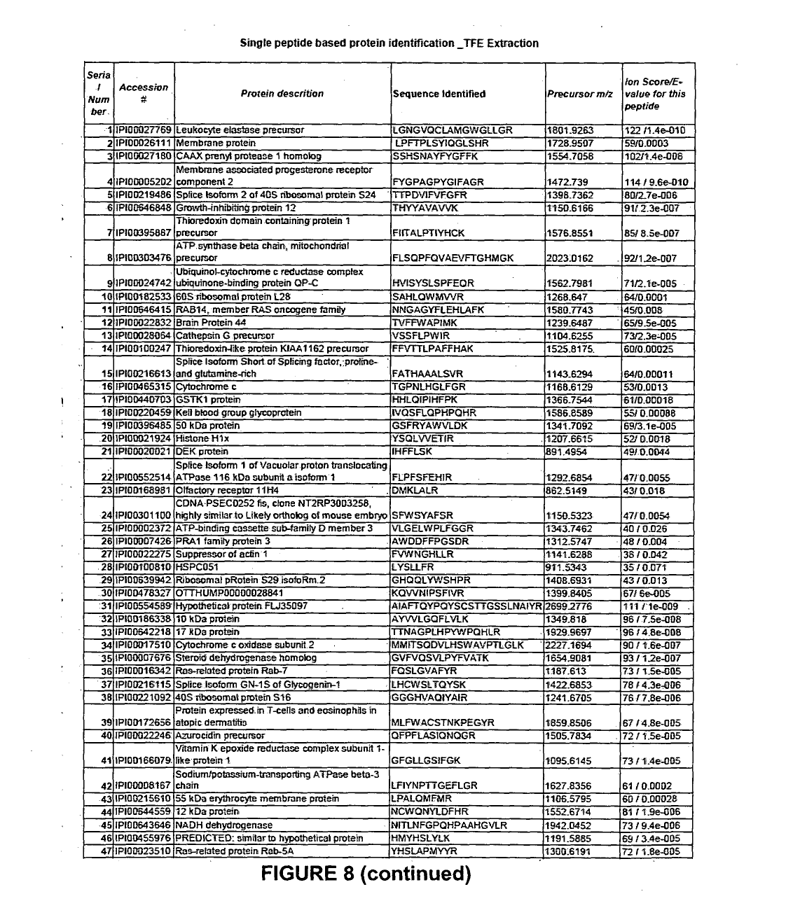

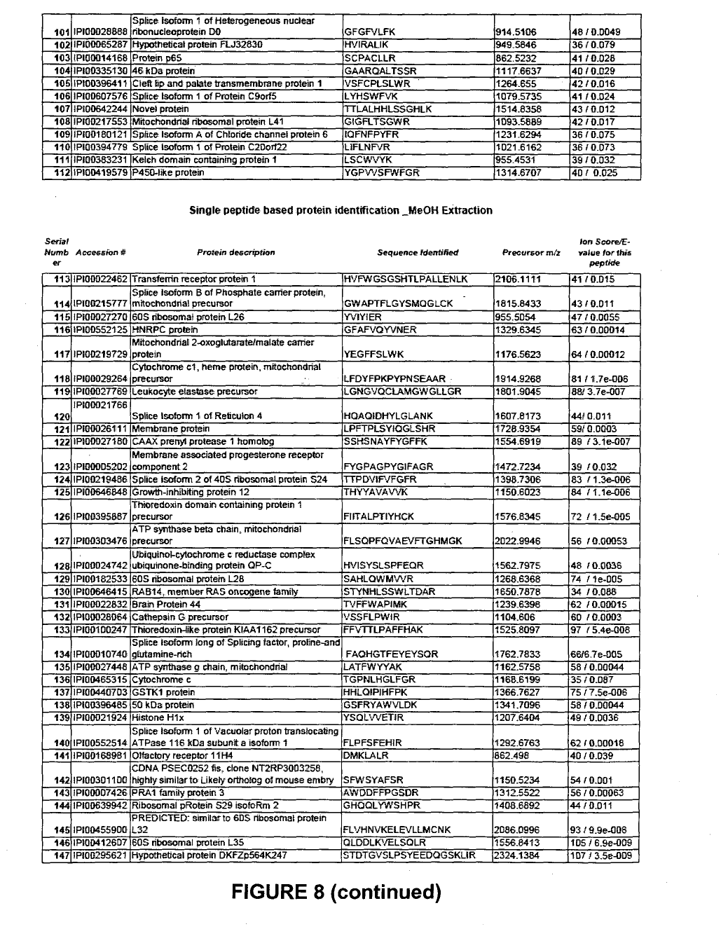

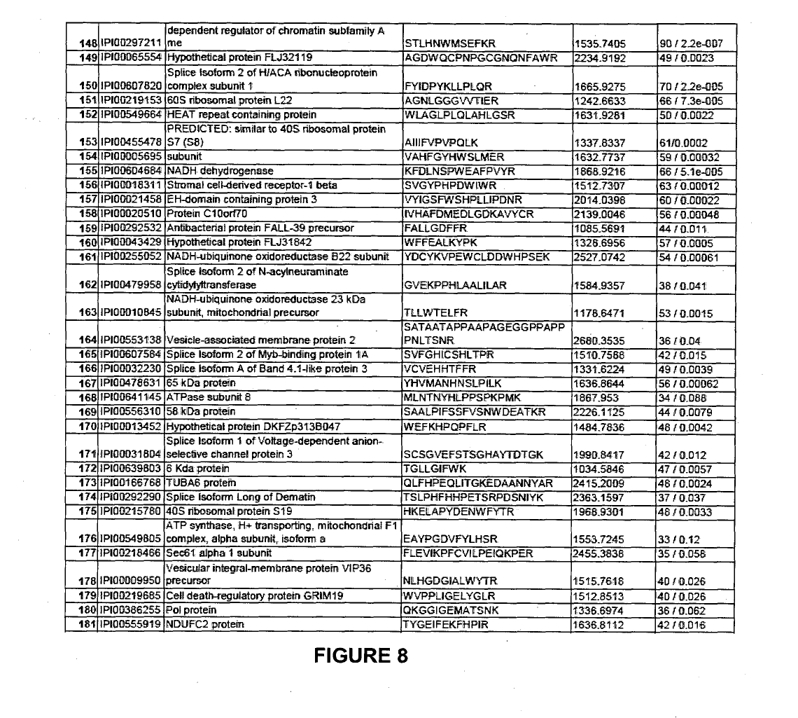

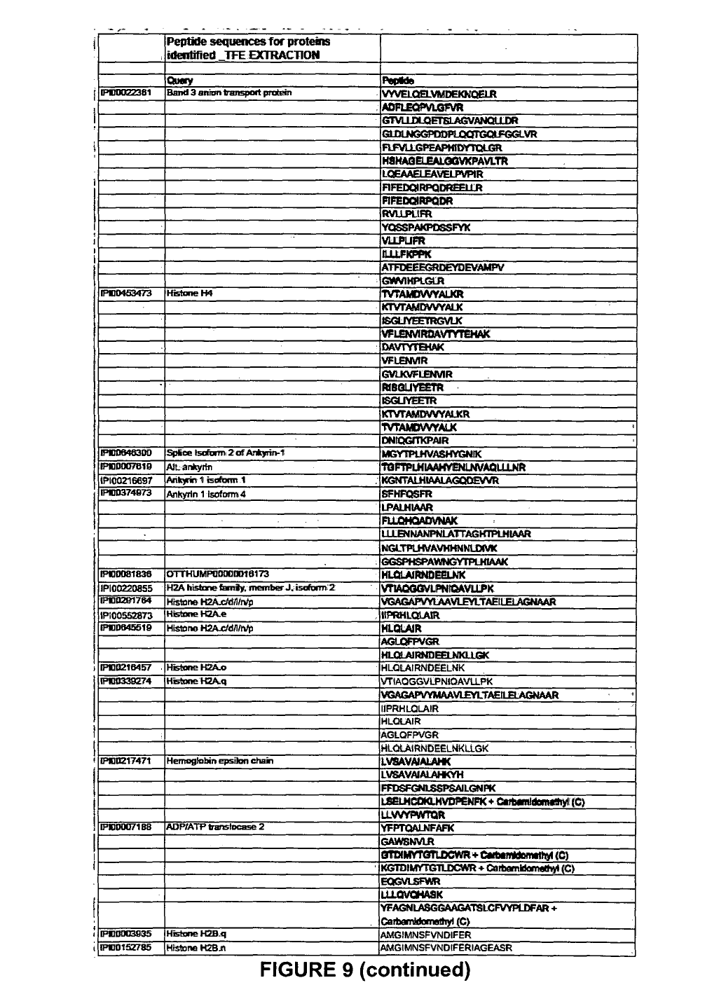

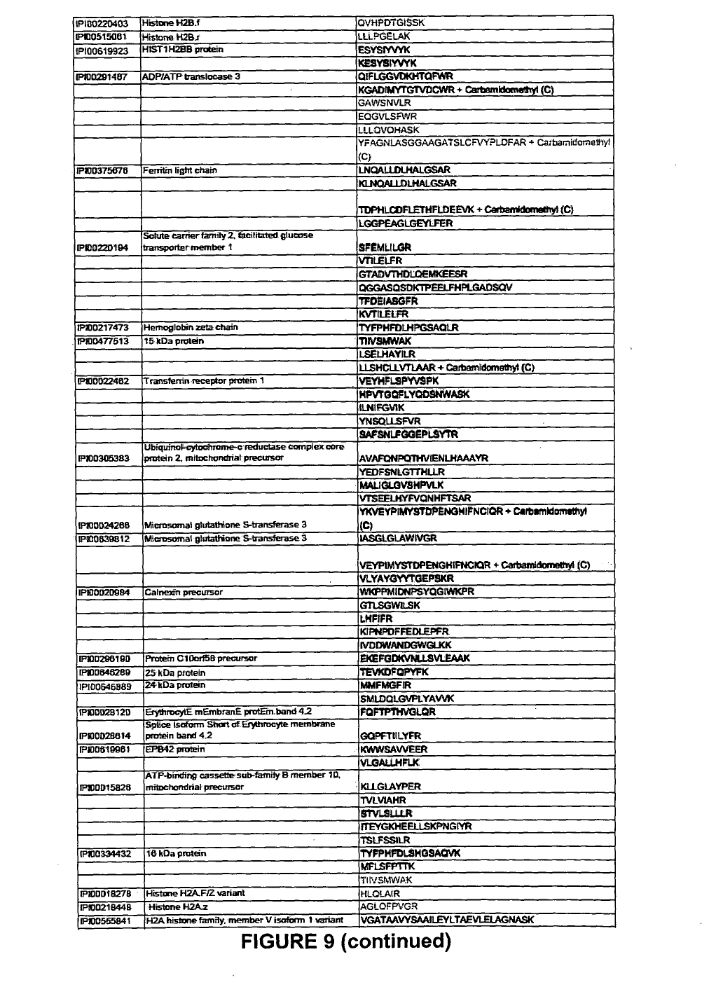

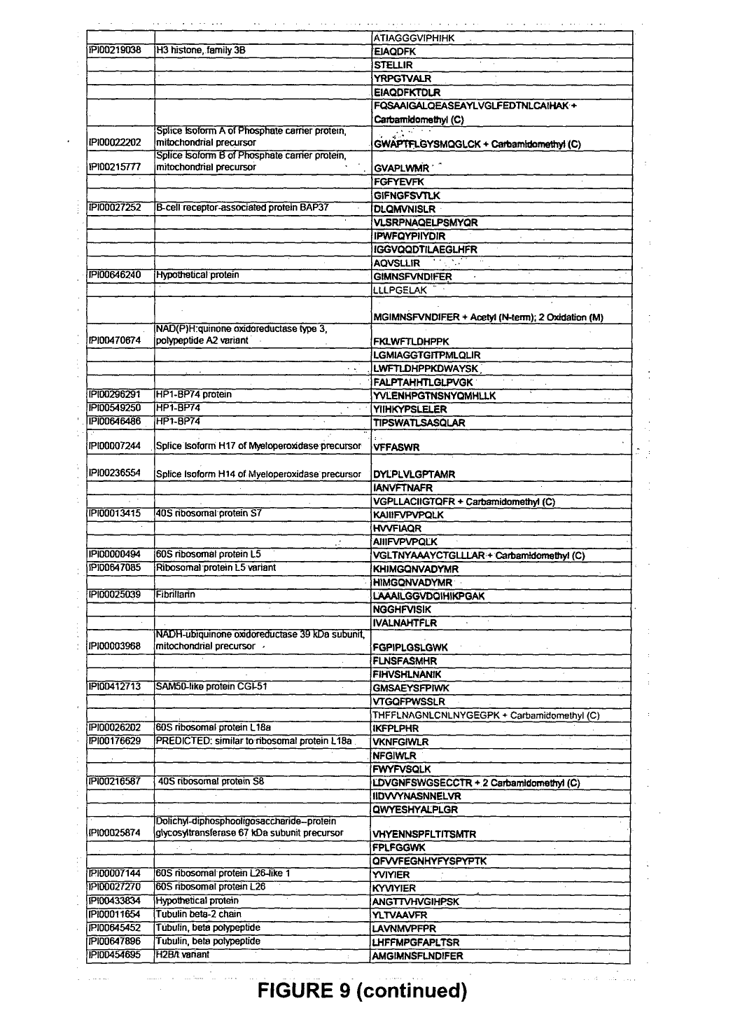

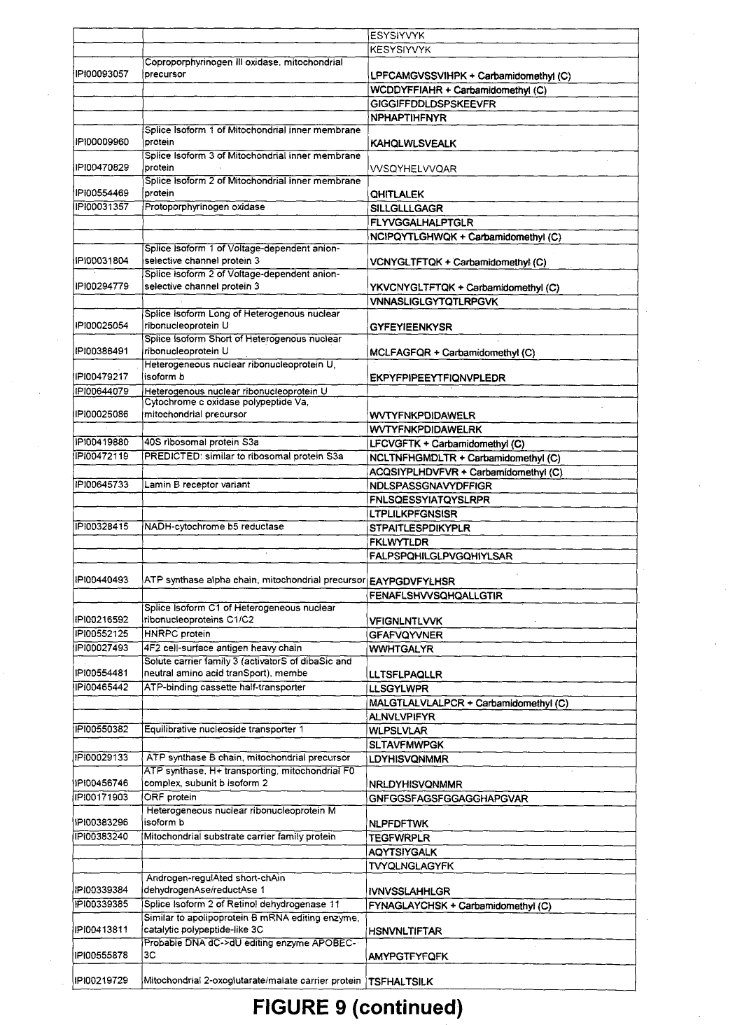

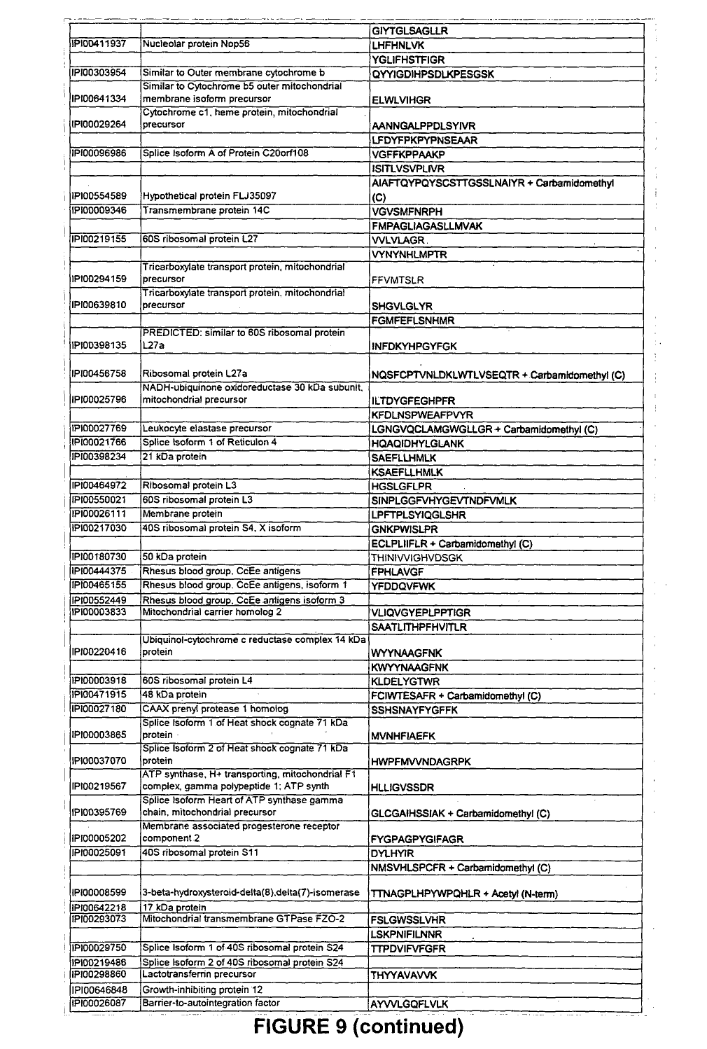

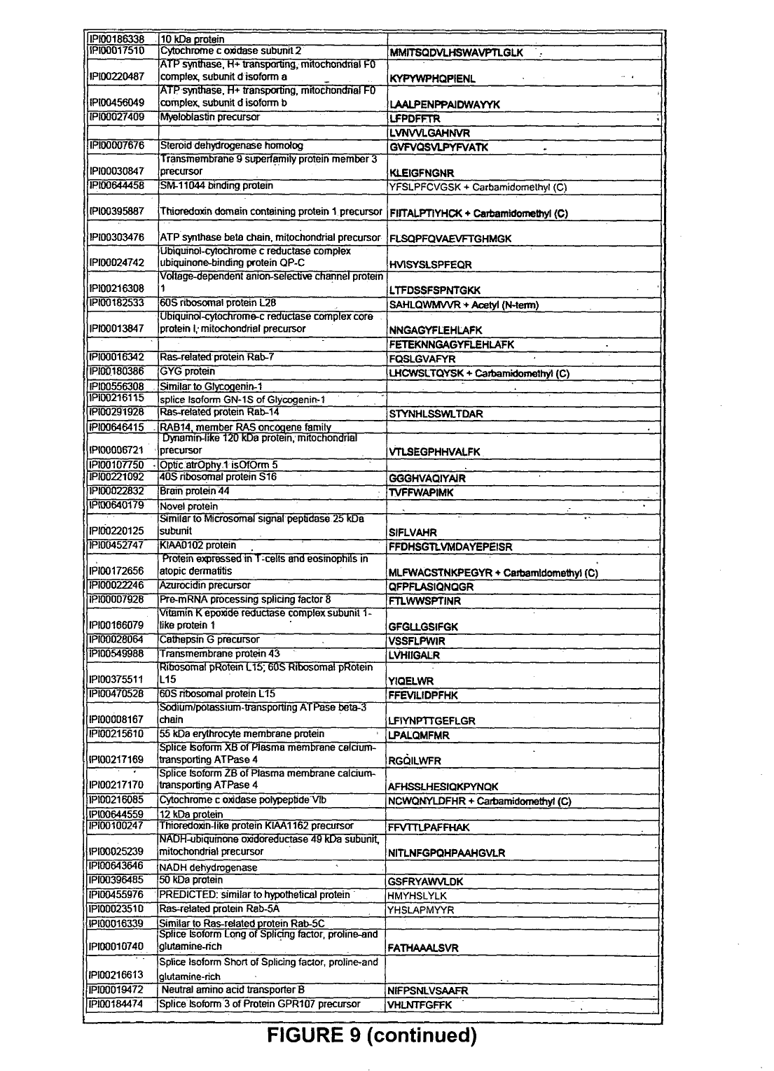

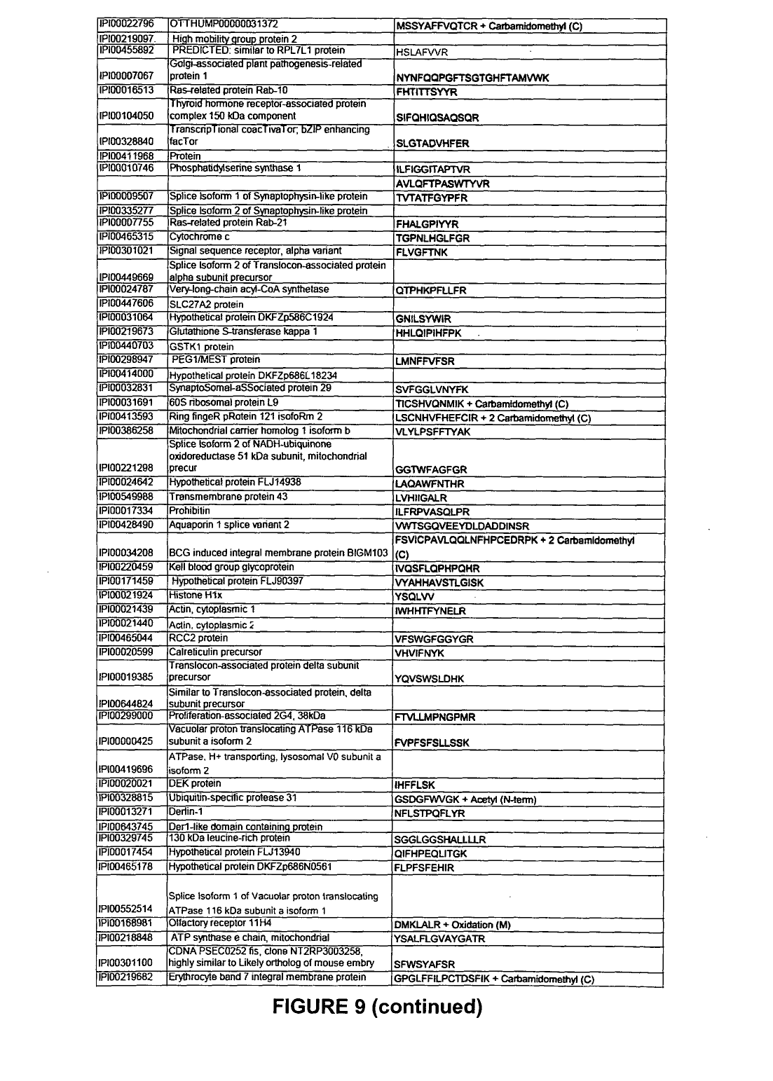

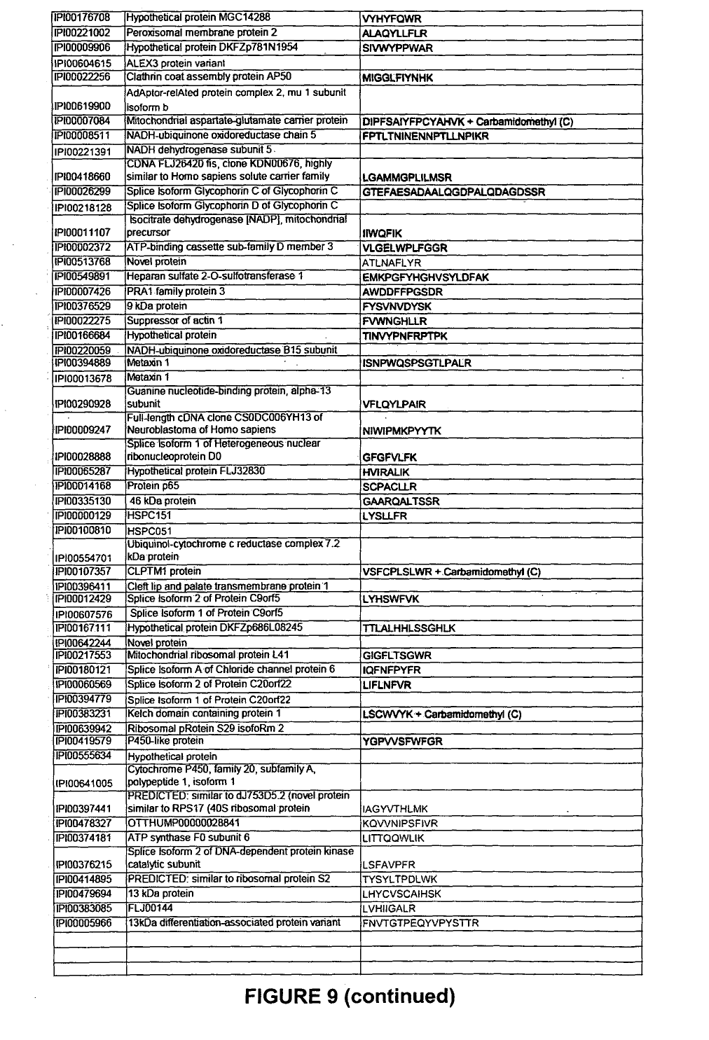

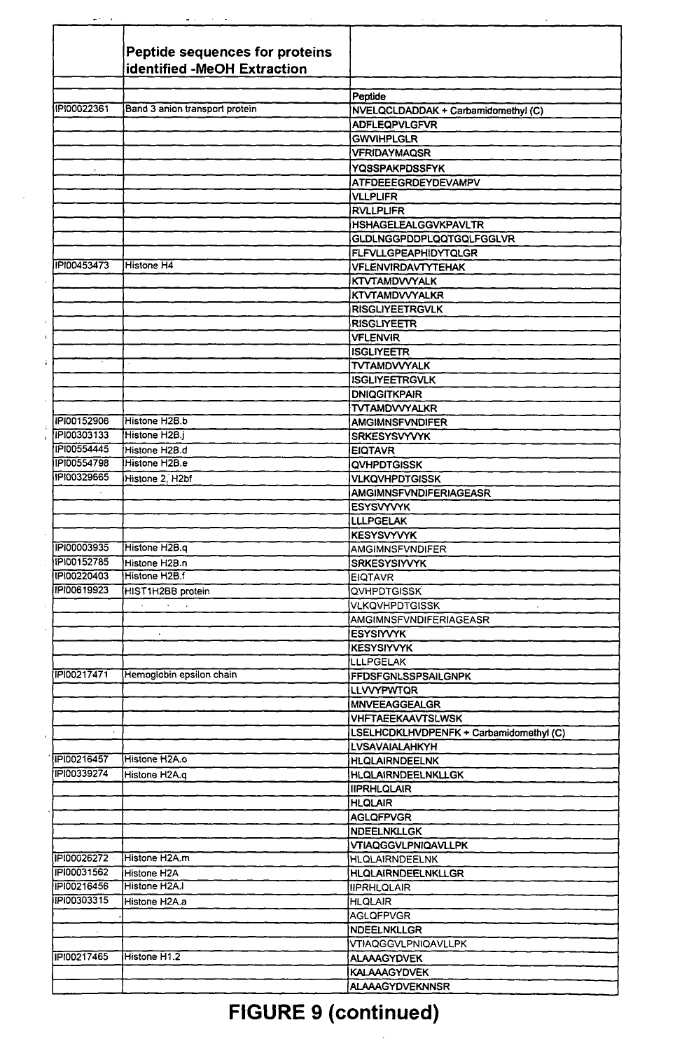

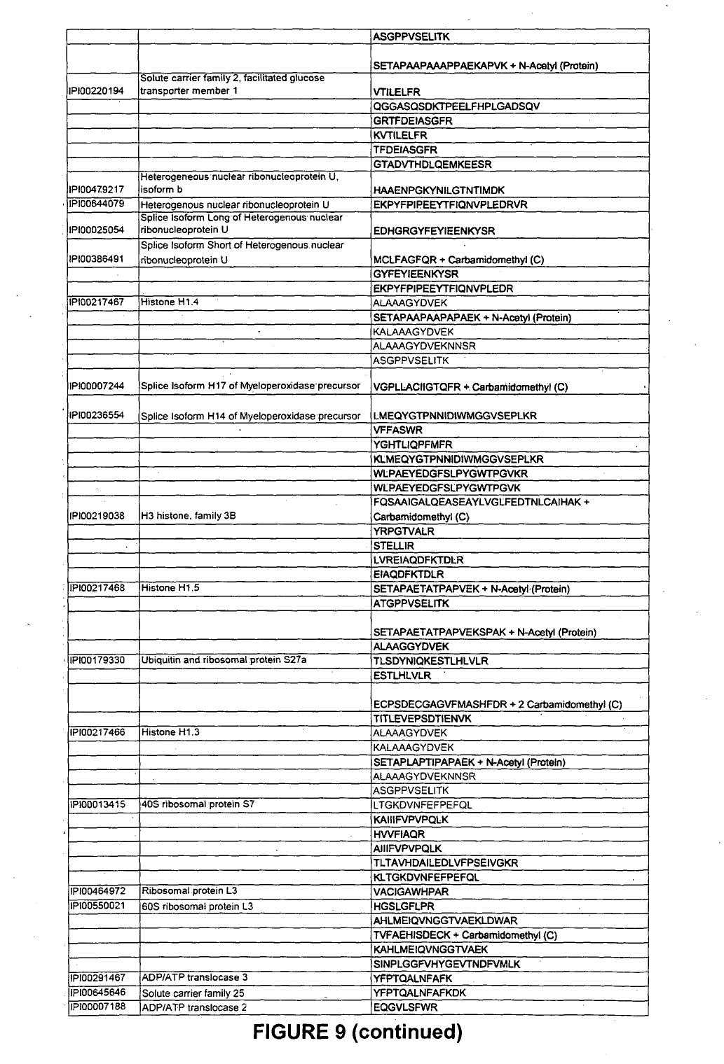

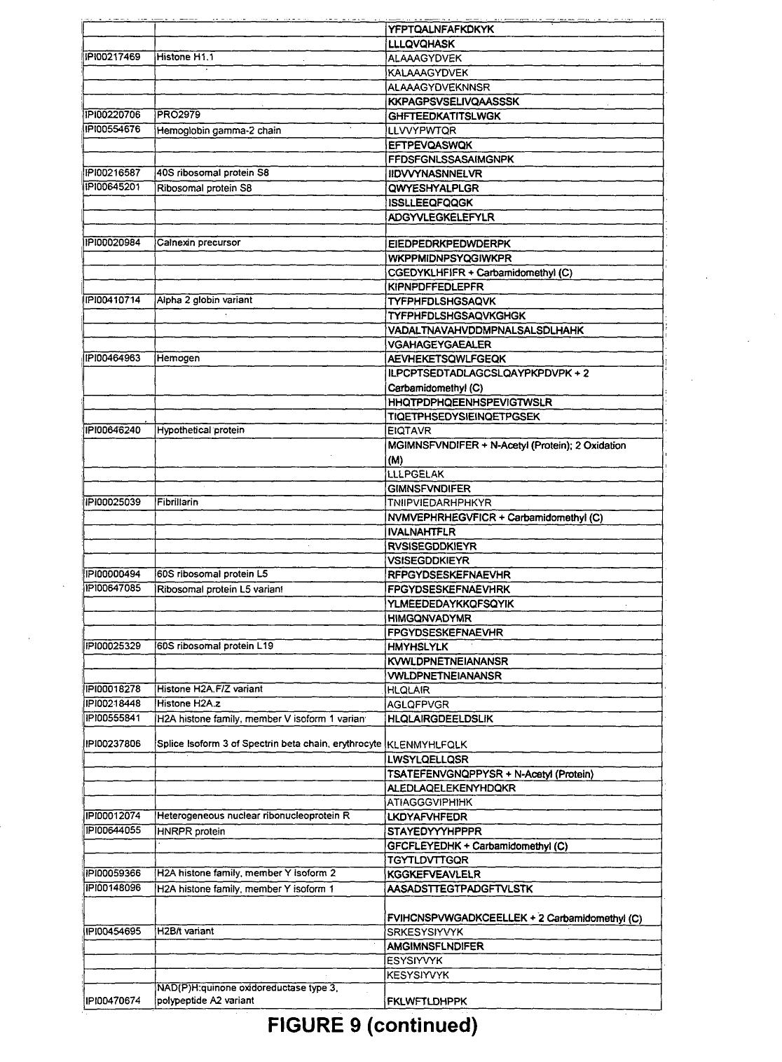

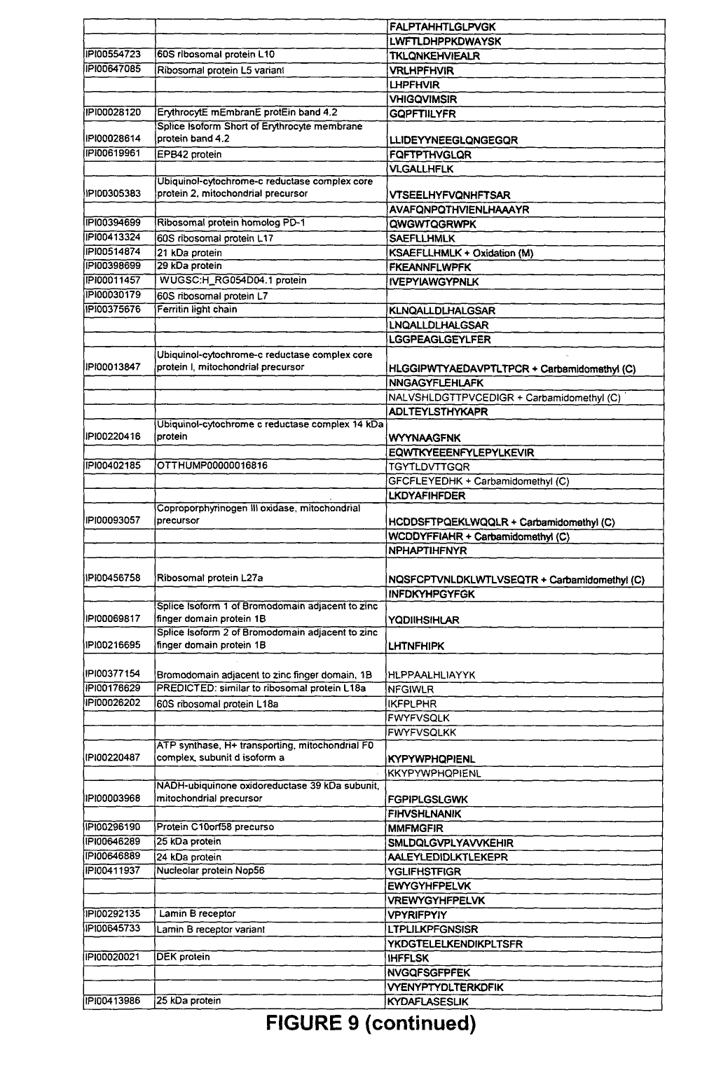

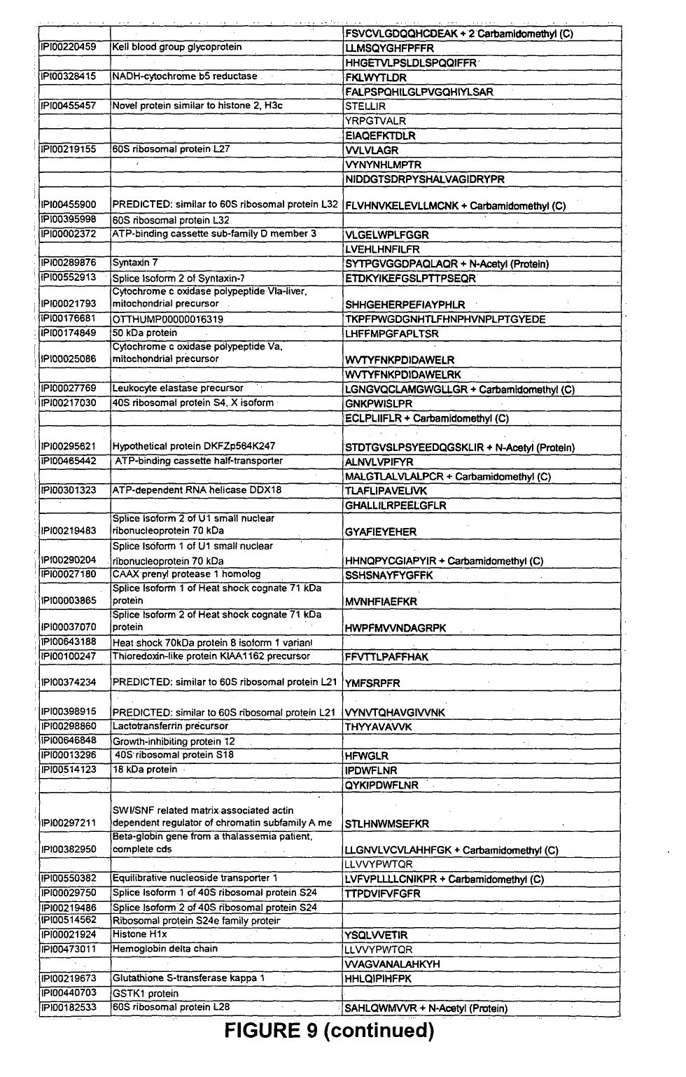

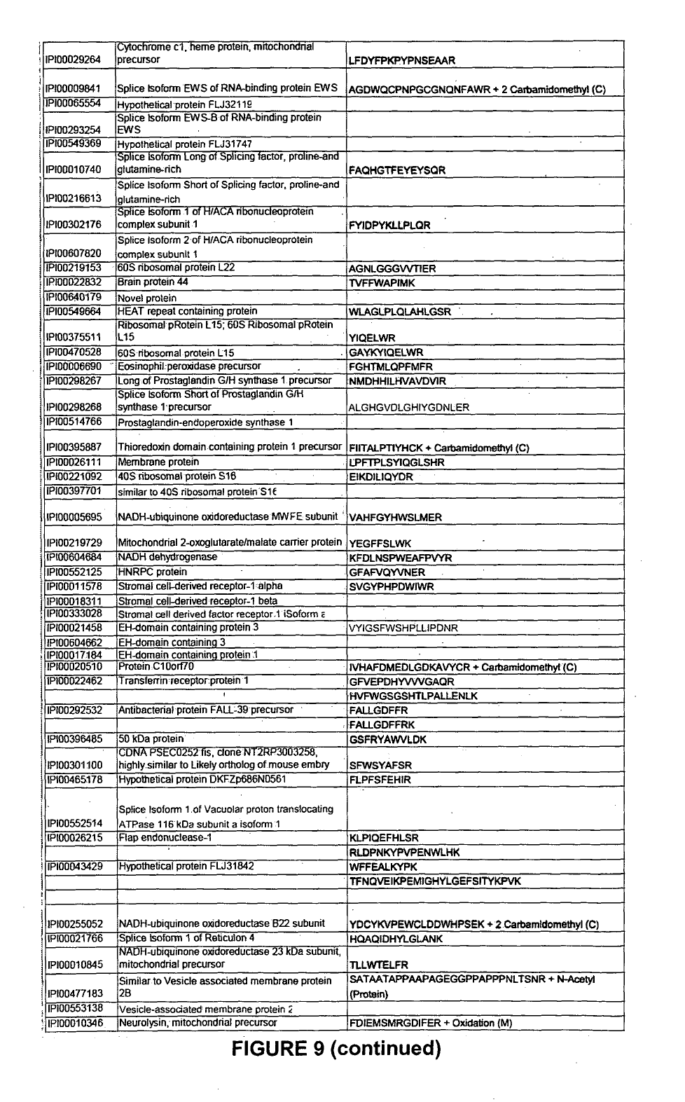

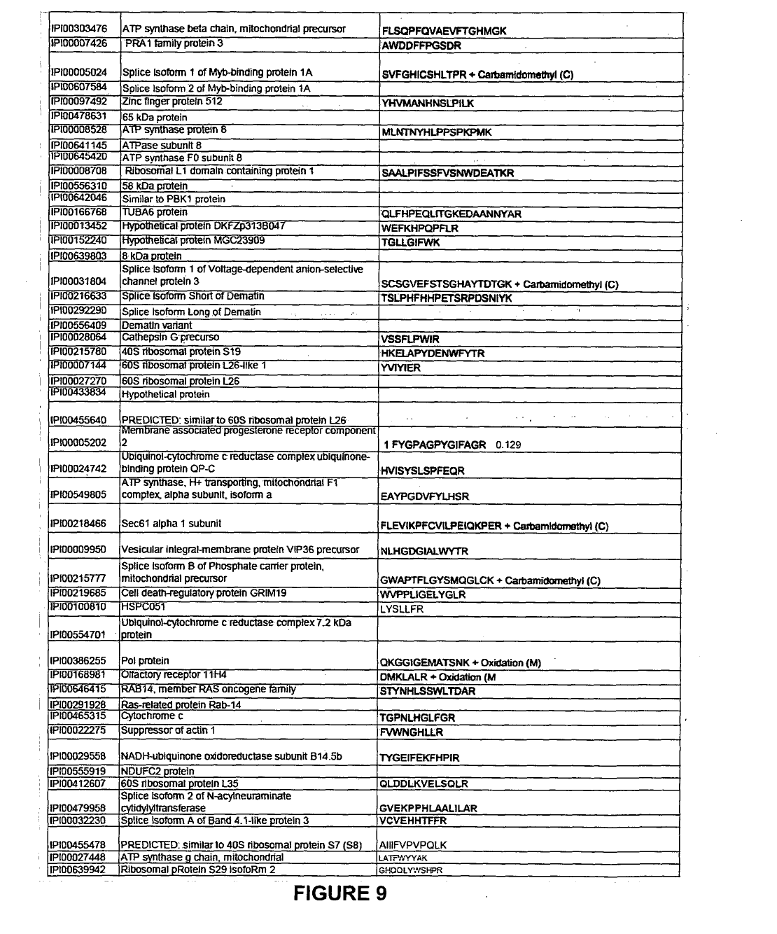

Colour coding of proteins based on the number of peptides for their identification shown in Table 2 indicated that only 23 of 273 total proteins were black coloured that were identified based on single peptide match which fall within the set threshold of 5% FDR, and the rest were red (≦2 peptides) or green coloured (by single peptide) where FDR was zero. Proteins identified based on single peptides from TFE and MeOH extractions, their peptide sequence and ion score are presented in FIGS. 7 and 8. Owing to the sample limitation of FPNRBCs, replicate mass-spectrometry analysis with more than the one pooled sample was not carried out.

Comparison of Plasma Membrane Proteins of FPNRBCs and AARBCs to Identify Unique Membrane Proteins

Mass spectrometry-based identification of membrane proteins of AARBCs have so far been reported by only a few studies including ours. From the published literature, a comprehensive list of all AARBC membrane proteins identified by mass spectrometry to date was curated. In the final list, only those candidates annotated as membrane proteins by gene ontology using GoFig. were included. Redundant entries were removed by manually comparing the sequences of all membrane proteins. A total of 299 non-redundant AARBC membrane proteins were finally short-listed (data not shown); Out of this, 202 were short-listed to include only membrane proteins with known- and potential surface domains (e.g. membrane-associated extracellular proteins and integral membrane proteins) (Table 8). Membrane proteins of FPNRBCs were compared manually with this final list of AARBC membrane proteins to identify both common and unique membrane proteins.

TABLE 6
Plasma membrane proteins of FPNRBCs

| No | Protein description | IPI Accession # | TMD | Sub-cellular location |
|---|---|---|---|---|
| 1 | Splice Isoform 1 of Protein C9orf5 | IPI00607576 | 14 | Plasma membrane |
| 2 | Solute carrier family 2, facilitated glucose transporter member 1 | IPI00220194 | 12 | Plasma membrane |
| 3 | Rhesus blood group, CcEe antigens, isoform 1 | IPI00465155 | 12 | Plasma membrane |
| 4 | Equilibrative nucleoside transporter 1 | IPI00550382 | 11 | Plasma membrane |
| 5 | Band 3 anion transport protein | IPI00022361 | 11 | Plasma membrane |
| 6 | ATP-binding cassette half-transporter | IPI00465442 | 9 | Plasma membrane |
| 7 | Neutral amino acid transporter B | IPI00019472 | 9 | Plasma membrane |
| 8 | Splice Isoform XB of Plasma membrane calcium-transporting | IPI00217169 | 8 | Plasma membrane |
| | ATPase 4 | | | |
| 9 | Olfactory receptor 11H4 | IPI00168981 | 7 | Plasma membrane |
| 10 | Splice Isoform 3 of Protein GPR107 precursor | IPI00184474 | 7 | Plasma membrane |
| 11 | BCG induced integral membrane protein BIGM103 | IPI00034208 | 7 | Plasma membrane |
| 12 | Sodium/potassium-transporting ATPase beta-3 chain | IPI00008167 | 1 | Plasma membrane |
| 13 | Hypothetical protein FLJ31842 | IPI00043429 | 6 | Plasma membrane |
| 14 | Cleft lip and palate transmembrane protein 1 | IPI00396411 | 5 | Plasma membrane |
| 15 | Splice Isoform A of Chloride channel protein 6 | IPI00180121 | 3 | Plasma membrane |
| 16 | Leukocyte elastase precursor | IPI00027769 | 1 | Plasma membrane |
| 17 | Solute carrier family 3 (activators of dibasic and neutral amino | IPI00554481 | 1 | Plasma membrane |
| | acid transport), member 2 | | | |
| 18 | Thioredoxin-like protein KIAA1162 precursor | IPI00100247 | 1 | Plasma membrane |
| 19 | Aquaporin 1 splice variant 2 | IPI00428490 | 1 | Plasma membrane |
| 20 | Kell blood group glycoprotein | IPI00220459 | 1 | Plasma membrane |
| 21 | Erythrocyte band 7 integral membrane protein | IPI00219682 | 1 | Plasma membrane |
| 22 | Splice Isoform Glycophorin D of Glycophorin C | IPI00218128 | 1 | Plasma membrane |
| 23 | Stromal cell-derived receptor-1 beta | IPI00018311 | 1 | Plasma membrane |
| 24 | Transferrin receptor protein 1 | IPI00022462 | 1 | Plasma membrane |
| 25 | Antibacterial protein FALL-39 precursor | IPI00292532 | 1 | Plasma membrane |

TABLE 7
Plasma membrane proteins of FPNRBCs Known to be present on other membranes

| No | Protein description | IPI Accession # | TMD | Sub-cellular location |
|---|---|---|---|---|
| 1 | CDNA PSEC0252 fis, clone NT2RP3003258, highly | IPI00301100 | 11 | Plasma membrane/ER Membrane |
| | similar to Likely ortholog of mouse embryo | | | |
| 2 | Splice isoform 1 of Vacuolar proton translocating | IPI00552514 | 7 | Plasma membrane/Vesicle membrane |
| | ATPase 116 kDa subunit a isoform 1 | | | |
| 3 | CAAX prenyl protease 1 homolog | IPI00027180 | 7 | Plasma membrane/ER/Golgi membrane |
| 4 | Splice Isoform 2 of Synaptophysin-like protein | IPI00335277 | 3 | Plasma membrane/Vesicle membrane |
| 5 | Microsomal glutathione S-transferase 3 | IPI00639812 | 3 | Plasma membrane/ER/Microsome |
| | | | | membrane |
| 6 | PRA1 family protein 3 | IPI00007426 | 3 | Plasma membrane/ER Membrane |
| 7 | Thioredoxin domain containing protein 1 precursor | IPI00395887 | 3 | Plasma membrane/ER Membrane |
| 8 | 17 kDa protein | IPI00642218 | 3 | Plasma membrane/ER Membrane |
| 9 | Splice isoform 1 of Reticulon 4 | IPI00021766 | 1 | Plasma membrane/ER Membrane |
| 10 | Suppressor of actin 1 | IPI00022275 | 2 | Plasma membrane/ER/Golgi membrane |
| 11 | Vesicle-associated membrane protein 2 | IPI00553138 | 1 | Plasma membrane/Vesicle |
| | | | | membrane/Synapse |
| 12 | Membrane associated progesterone receptor | IPI00005202 | 1 | Plasma membrane/Microsome membrane |
| | component 2 | | | |
| 13 | Vesicular integral-membrane protein VIP36 precursor | IPI00009950 | 1 | Plasma membrane/ER/Golgi membrane |
| 14 | Calnexin precursor | IPI00020984 | 1 | Plasma membrane/ER Membrane |

TABLE 8
Comprehensive AARRBC membrane proteins with potential surface domain(s)
(Adjacent proteins in colour were identified from same peptide but in different studies,
and were counted as one protein)

| No. | Accession # | Protein description | TMD | Subcellular localisation |
|---|---|---|---|---|
| 1 | IPI00514990 | 101 kDa protein | 8 | Integral membrane protein |
| 2 | IPI00642218 | 17 kDa protein | 3 | Integral membrane protein |
| 3 | IPI00478755 | 22 kDa protein | 0 | Plasma membrane |
| 4 | IPI00641837 | 27 kDa protein | 3 | Integral membrane protein |
| 5 | IPI00069985 | 28 kDa protein | 1 | Integral membrane protein |
| 6 | IPI00293895 | ABC transporter ABCA7 | 11 | Integral membrane protein |
| 7 | IPI00220026 | Acetylcholinesterase precursor/ACHE protein | 0 | Membrane; extracellular |
| 8 | IPI00026103 | Splice Isoform 1 of P22303 Acetylcholinesterase | 0 | Integral membrane protein |
| | | precursor | | |
| 9 | IPI00296333 | Acyl-Coa synthetase long-chain family member 6 | 1 | Membrane |
| | | isoform A | | |
| 10 | IPI00031131 | Adipocyte plasma membrane-associated protein, | 1 | Membrane associated protein |
| | | Low molecular weight phosphotyrosine protein | | |
| | | phosphatase | | |
| 11 | IPI00395006 | Splice Isoform 2 of ADP-ribosyl cyclase 1 | 1 | Integral membrane protein |
| 12 | IPI00006608 | Splice Isoform APP770 of P05067 Amyloid beta A4 | 1 | Type I membrane protein |
| | | protein precursor | | |
| 13 | IPI00001856 | Annexin A11 protein | 1 | Membrane associated protein |
| 14 | IPI00021842 | Apolipoprotein E precursor | 0 | Extracellular binding RBC |
| 15 | IPI00024689 | Aquaporin 1 | 6 | Integral membrane protein |
| 16 | IPI00428490 | Aquaporin 1 splice variant 2 | 1 | Inteqral MP |
| 17 | IPI00465442 | ATP-bindinq cassette half-transporter | 9 | Integral membrane protein; Inner |
| | | | | mitochondrial |
| | IPI00014555 | ATP-binding cassette, sub-family B, member 6, | 9 | Mitochondrial membrane |
| | | mitochondrial precursor | | |
| 18 | Gi6715561 | ATP-bindinq cassette, subfamily C, member 6 | 12 | Integral membrane protein |
| 19 | IPI00008463 | ATP-binding cassette, sub-family C, member 1 | 15 | Integral membrane prolein |
| | | isoform 6 | | |
| 20 | IPI00298214 | ATP-binding cassette, sub-family G, member 2 | 6 | Integral membrane protein |
| 21 | IPI00022361 | Band 3 anion transport protein | 11 | Integral membrane prolein |
| 22 | IPI00019906 | Splice isoform 2 or 1 of P35613 Basigin precursor | 2 | Type I membrane protein |
| 23 | IPI00418163 | C4B1 | 0 | Extracellular |
| 24 | IPI00020984 | Calnexin precursor | 1 | Type I membrane protein |
| 25 | IPI00020599 | Calreticulin precursor | 0 | ER: Extracellular, Cytosolic protein |
| 26 | IPI00032038 | Camitine O-palmitoyltransferase I, mitochondrial liver | 2 | Mitochondrial outer membrane |
| | | isoform | | |
| 27 | IPI00028064 | Cathepsin G precursor | 0 | Extracellular binding RBC |
| 28 | IPI00297160 | CD44 antigen | 1 | Type I membrane protein |
| | Gi7512338 | Cell surface glycoprotein CD44 | 1 | Integral membrane protein |
| | IPI00305064 | Splice Isoform CD44 of CD44 antigen precursor | 1 | Integral membrane protein |
| 29 | IPI00011302 | CD59 glycoprotein precursor | 0 | Membrane associated protein |
| 30 | Gi1314306 | Channel-like integral membrane protein | 3 | integral membrane protein |
| 31 | IPI00221393 | Splice isoform 1 of Choline transporter-like protein 1 | 9 | Integral membrane prolein |
| 32 | IPI00549521 | Splice Isoform 1 of Choline transporter-like protein 2 | 11 | Integral membrane protein |
| 33 | IPI00219677 | CGI-26 protein | 0 | Extracellular |
| 34 | IPI00291262 | Clusterin precursor | 0 | Extracellular binding RBC |
| 35 | IPI00216550 | Splice Isoform 1 ot P08174 Complement decay- | 0 | Membrane associated protein |
| | | accelerating factor precursor | | |
| | IPI00292069 | Splice Isoform 2 of Complement decay-accelerating | 0 | Membrane associated protein |
| | | factor precursor | | |
| 36 | IPI00164623 | Complement C3b | 0 | Extracellular binding RBC |
| 37 | IPI00640083 | Complement component (3b/4b) receptor 1, incl. | 1 | Integral membrane protein |
| | | Knops blood group | | |
| | IPI00412546 | Complement receptor 1 | 1 | Integral membrane protein |
| | IPI00018287 | Complement receptor type I precursor | 1 | Type I membrane protein |
| 38 | IPI00016608 | Cop-coated vesicle membrane protein p24 precursor | 2 | Integral membrane protein |
| 39 | IPI00028610 | Splice Isoform 4 of Q04656 Copper-transporting | 7 | Golgi, Plasma membrane |
| | | ATPase 1 | | |
| 40 | IPI00023780 | Splice Isoform 2 or 1 of Q9H3Z4 DNAJ homolog | 1 | Membrane associated protein |
| | | subfamily C member 5 | | |
| 41 | IPI00165394 | DC-TM4F2 protein | 4 | Integral membrane protein |
| 42 | IPI00550523 | DKFZP564J0863 protein | 2 | Unclassified |
| 43 | IPI00154755 | Down syndrome cell adhesion molecule 2 | 1 | Type 1 membrane protein |
| 44 | IPI00215964 | Splice Isoform 1 of Duffy antigen/chemokine receptor | 7 | Integral membrane protein |
| | IPI00002940 | Splice Isoform 2 of Q16570 Duffy antigen/chemokine | 7 | Integral membrane protein |
| | | receptor | | |
| 45 | IPI00432050 | Duodenal cytochrome b sequence coverage: 10% | 1 | Integral membrane protein |
| | Gi13376257 | Duodenal cytochrome b | 6 | Integral membrane protein |
| 46 | IPI00004065 | Ecto-ADP-ribosyltransferase 4 precursor | 0 | Integral membrane protein |
| 47 | IPI00010341 | Eosinophil granule major basic protein precursor | 0 | Extracellular |
| 48 | IPI00550382 | Equilibrative nucleoside transporter 1 | 11 | Integral membrane protein |
| 49 | IPI00647116 | Erythroblast membrane-associated protein | 1 | Integral membrane protein |
| 50 | IPI00044556 | Erythroid membrane-associated protein | 2 | Membrane associated protein |
| 51 | IPI00302538 | EVIN2 | 8 | Integral membrane protein |
| 52 | IPI00216890 | Similar to expressed sequence AA536743 | 2 | Integral membrane protein |
| 53 | IPI00022418 | Splice Isoform 1 Of Fibronectin precursor | 0 | Integral membrane protein; Extracellular |
| 54 | IPI00221205 | Galactosylgalactosylxylosylprotein 3-beta- | 1 | Integral membrane protein; Golgi |
| | | glucuronosyltransferase 2 | | |
| 55 | IPI00465431 | Galectin-3 | 0 | Extracellular binding RBC |
| 56 | IPI00010477 | Splice Isoform Long of O00182 Galectin-9 | 0 | Extracellular |
| 57 | IPI00306419 | gene rich cluster, C3f gene | 7 | Unclassified |
| 58 | IPI00298800 | Glycophorin A precursor | 2 | Type I membrane protein |
| | Gi13529077 | Similar to Glycophorin A | 2 | Type I membrane protein |
| 59 | Gi106140 | Glycophorin A | 2 | Type I membrane protein |
| 60 | IPI00384414 | Glycophorin Erik I-IV precursor | 1 | Integral membrane protein |
| 61 | Gi4504229 | Glycophorin C, isoform 1 | 1 | Integral membrane protein |
| | IPI00026299 | Splice Isoform Glycophorin C of P04921 Glycophorin | 1 | Integral membrane protein |
| | | C | | |
| | IPI00218128 | Splice Isoform Glycophorin D of Glycophorin C | 1 | Integral membrane protein |
| 62 | IPI00023542 | Gp25L2 protein | 2 | Integral membrane protein |
| 63 | Gi9295192 | HGTD-P | 1 | Integral membrane protein |
| 64 | gi18552304 | Hypothetical protein XP 092517 | 1 | Integral membrane protein |
| 65 | IPI00029002 | Hypothetical protein | 4 | Integral membrane protein |
| 66 | IPI00031697 | Hypothetical protein | 5 | Unclassified |
| 67 | IPI00032825 | Hypothetical protein CGI-109 precursor | 1 | Integral membrane protein |
| 68 | IPI00383828 | Hypothetical protein DKFZp564J0863 | 2 | Unclassified |
| 69 | IPI00178934 | Hypothetical protein 327024.1 | 1 | Integral membrane protein |
| 70 | IPI00030236 | Hypothetical protein DKFZp564D0478 | 3 | Integral membrane protein |
| 71 | IPI00100199 | Hypothetical protein DKFZp564E227 | 6 | Integral membrane protein |
| 72 | IPI00032013 | Hypothetical protein DKFZp762A227 | 11 | Integral membrane protein |
| 73 | IPI00022300 | Hypothetical protein FLJ14347 | 1 | Unclassified |
| 74 | IPI00442030 | Hypothetical protein FLJ16766 | 7 | Integral membrane protein |
| 75 | IPI00043429 | Hypothetical protein FLJ31842 | 6 | Integral membrane protein |
| 76 | IPI00167359 | Hypothetical protein FLJ40269 | 2 | Inteqral membrane protein |
| 77 | IPI00171004 | Hypothetical protein MGC34680 | 12 | Integral membrane protein |
| 78 | IPI00003441 | Hypothetical protein ORF9 precursor | 1 | Integral membrane protein |
| 79 | IPI00171421 | Hypothetical protein PSEC0098 | 1 | Unclassified |
| 80 | IPI00332161 | Ig gamma-1 chain C region | 0 | Extracellular binding RBC |
| 81 | IPI00385058 | Ig kappa chain C region | 0 | Extracellular binding RBC |
| 82 | Gi87863 | Ig heavy chain V-V region | 0 | Extracellular |
| 83 | P05107 | Integrin beta-2 precursor | 1 | Integral membrane protein |
| 84 | IPI00000118 | Splice Isoform Long of Intercellular adhesion | | |
| | | molecule-4 precursor | 1 | integral membrane protein |
| | IPI00396335 | Splice Isoform Short of Intercellular adhesion | 0 | Membrane |
| | | molecule-4 precursor | | |
| 85 | Gi2134798 | B-CAM protein | 1 | Integral membrane protein |
| 86 | IPI00032466 | Intermediate conductance calcium-activated | 5 | Integral membrane protein |
| | | potassium channel protein 4 | | |
| 87 | IPI00415077 | Ion transporter protein | 9 | Integral membrane protein |
| 88 | IPI00001754 | Junctional adhesion molecule 1 precursor | 2 | Type I membrane protein |
| 89 | IPI00007426 | JWA protein regulates intracellular concentrations of | 3 | Integral membrane protein |
| | | taurine and glutamate | | |
| 90 | IPI00220459 | Kell blood group glycoprotein | 1 | Integral membrane protein |
| 91 | IPI00001952 | KIAA0830 protein | 3 | Integral membrane protein |
| 92 | IPI00022275 | KIAA0851 protein | 2 | Integral membrane protein |
| 93 | IPI00002230 | KIAA1363 protein | 1 | Integral membrane protein |
| 94 | IPI00298860 | Lactotransferrin precursor | 0 | Extracellular binding RBC |
| 95 | P42702 | Leukemia inhibitory factor receptor | 1 | Integral membrane protein |
| 96 | IPI00027769 | Leukocyte elastase precursor | 1 | Extracellular |
| 97 | IPI00216514 | Splice Isoform OA3-293 of Leukocyte surface antigen | 0 | Integral membrane protein |
| | | CD47 precursor | | |
| | IPI00374740 | Splice Isoform OA3-323 of Leukocyte surface antigen | 6 | Intearal membrane protein |
| | | CD47 precursor | | |
| 98 | IPI00000059 | LFA-3 | 2 | Integral membrane protein |
| 99 | IPI00031397 | Lono-chom-fatty-acid--CoA ligase 3 | 1 | Type III membrane protein |
| 100 | IPI00218718 | Splice Isoform Short of Q9UKU0 Long-chain-fatty- | 0 | Type III membrane protein |
| | | acid--CoA ligase 6 | | |
| 101 | IPI00023858 | Low affinity immunoglobulin gamma Fc region | 1 | Integral membrane protein |
| | | receptor III-B precursor | | |
| 102 | IPI00002406 | Lutheran blood group glycoprotein precursor | 1 | Type I membrane protein |
| | IPI00328869 | Lutheran blood group | 1 | Integral membrane protein |
| | IPI00554618 | Lutheran blood group glycoprotein isoform 2 | 1 | Integral membrane protein |
| | | precursor | | |
| 103 | Gi18589892 | Similar to Lutheran blood group | 0 | Intearal membrane protein |
| 104 | IPI00219549 | Splice Isoform Short of Lymphocyte function- | 2 | Integral membrane protein |
| | | associated antigen 3 precursor | | |
| 105 | IPI00019038 | Lysozyme C precursor | 0 | Extracellular |
| 106 | IPI00005202 | Membrane associated progesterone receptor | 1 | Integral membrane protein |
| | | component 2 | | |
| 107 | IPI00026111 | Membrane protein | 2 | Unclassified |
| 108 | IPI00020896 | Membrane transport protein XK | 9 | Intearal membrane protein |
| 109 | IPI00010292 | Mesenchymal stem cell protein DSCD75 | 1 | Unclassified |
| 110 | IPI00639812 | Microsomal glutathione S-transferase 3 | 3 | Intearal membrane protein; Microsome |
| 111 | IPI00024650 | Monocarboxylate transporter 1 | 11 | Integral membrane protein |
| 112 | IPI00008338 | Splice Isoform Delexon-17 of P33527 Multidrug | 16 | Integral membrane protein |
| | | resistance-associated protein 1 | | |
| 113 | IPI00006675 | Multidrug resistance-associated protein 4 | 11 | Intearal membrane protein |
| 114 | IPI00385383 | Multidrug resistance-associated protein 5 | 11 | Integral membrane protein |
| 115 | IPI00027409 | Myeloblastin precursor | 0 | Extracellular |
| 116 | IPI00021983 | Splice isoform 1 of Q92542 Nicastrin precursor | 0 | Type I membrane protein |
| 117 | IPI00217600 | Neuropathy target esterase | 1 | Unclassified |
| 118 | IPI00011454 | Splice Isoform 2 of Q14697 Neutral alpha- | 1 | ER; Golgi |
| | | glucosidase AB precursor | | |
| 119 | IPI00479732 | Splice Isoform 2 of Large neutral amino acids | 11 | Integral membrane protein |
| | | transporter small subunit 3 | | |
| 120 | IPI00513701 | Novel protein | 3 | Integral membrane protein |
| 121 | IPI00009507 | Splice isoform 1 of Q16563 Pantophysin | 3 | Integral membrane protein |
| 122 | IPI00021075 | PB39 | 12 | Integral membrane protein |
| 123 | IPI00020124 | Phosphatidylinositol 4-kinase type II | 0 | Intearal membrane protein |
| 124 | IPI00009688 | Phosphatidylinositol-4-phosphate 5-kinase type II | 0 | Integral membrane protein |
| | | alpha | | |
| | Gi1730569 | Phosphatidylinositol-4-phosphate 5 kinase, type III | 0 | Integral membrane protein |
| 125 | IPI00005181 | Phospholipid scramblase 1 | 0 | Type II membrane protein |
| 126 | IPI00016776 | Phospholipid scramblase 4 | 0 | Type II membrane protein |
| 127 | IPI00021695 | Splice Isoform B of P20020 Plasma membrane | 7 | Integral membrane protein |
| | | calcium-transporting ATPase 1 | | |
| 128 | IPI00217169 | Splice Isoform XB of Plasma membrane calcium- | 8 | Integral membrane protein |
| | | transporting ATPase 4 | | |
| | IPI00012490 | Splice Isoform XD of P23634 Plasma membrane | 8 | Intearal membrane protein |
| | | calcium-transporting ATPase 4 | | |
| 129 | IPI00003648 | Splice Isoform Delta of Poliovirus receptor related | 2 | Integral membrane protein |
| | | protein 1 precursor | | |
| 130 | IPI00024670 | Polyposis locus protein 1 | 2 | Integral membrane protein |
| 131 | IPI00029507 | Potassium channel subfamily K member 5 | 6 | Integral membrane protein |
| 132 | Gi6409316 | Presenilin-associated protein | 2 | Integral membrane protein |
| 133 | IPI00022974 | Prolactin | 0 | Extracellular binding RBC |
| 134 | IPI00033075 | Protein BAT5 | 2 | Integral membrane protein |
| 135 | IPI00010796 | Protein disulfide-isomerase precursor | 0 | ER lumen, extracellular region |
| 136 | IPI00006093 | Protein FAM38A | 25 | Unclassified |
| 137 | IPI00006072 | Protein transport protein SEC61 gamma subunit | 1 | Integral membrane protein |
| 138 | IPI00290452 | RECS1 protein homolog | 7 | Integral membrane protein |
| 139 | IPI00028946 | Reticulon protein 3 | 3 | Integral membrane protein |
| | IPI00555783 | Reticulon 3 isoform a variant | 3 | Membrane: Extracellular |
| | IPI00398795 | RTN3-A1 | 3 | Integral membrane protein |
| | IPI00177423 | PREDICTED: similar to Reticulon protein 3 | 1 | Integral membrane protein |
| 140 | IPI00298289 | Splice Isoform 2 Of Reticulon 4 | 1 | Integral membrane protein |
| 141 | IPI00039665 | Rh blood CE group antigen polypeptide | 12 | Integral membrane protein |
| 142 | IPI00329565 | RhD protein | 10 | Integral membrane protein |
| | IPI00478119 | Rhesus blood group D antigen | 10 | Integral membrane protein |
| | Gi10800054 | Rh blood D group antigen polypeptide | 10 | Integral membrane protein |
| 143 | Gi2765839 | Rhesus D category VI type III protein | 12 | Integral membrane protein |
| 144 | IPI00024094 | Rhesus blood group-associated glycoprotein | 11 | Integral membrane protein |
| 145 | IPI00465155 | Rhesus blood group, CcEe antigens, isoform 1 | 12 | Integral membrane protein |
| 146 | IPI00221017 | Splice Isoform RHVIII of P18577 Blood group | 10 | Integral membrane protein |
| | | Rh(CE) polypeptide | | |
| 147 | IPI00444375 | Hypothetical protein FLJ45640 (Rhesus blood group, | 10 | Integral membrane protein |
| | | CcEe antigens) | | |
| 148 | IPI00166865 | Similar to RIKEN cDNA 1500009M05 gene | 1 | Unclassified |
| 149 | IPI00373867 | PREDICTED: similar to RIKEN cDNA C730027E14 | 1 | Integral membrane protein |
| 150 | IPI00056310 | Secretory carrier-associated membrane protein 4 | 4 | Integral membrane protein |
| 151 | IPI00025257 | Semaphorin 7A precursor | 0 | Integral membrane protein |
| 152 | IPI00022434 | Serum albumin precursor | 0 | Extracellular |
| 153 | IPI00219755 | Signal peptidase complex subunit 1 | 2 | Integral membrane protein |
| 154 | Q64689 | Alpha-2,8-sialyltransferase 8C | 1 | Inner cell membrane |
| 155 | IPI00216029 | Splice Isoform 2 of Sodium channel protein type I | 19 | Integral membrane protein |
| | | alpha subunit | | |
| 156 | IPI00006482 | Splice Isoform of Sodium/potassium-transporting | 10 | Integral membrane protein |
| | | ATPase alpha-1 chain | | |
| 157 | IPI00003021 | Sodium/potassium-transporting ATPase alpha-2 | 8 | Integral membrane protein |
| | | chain precursor | | |
| 158 | IPI00414005 | Splice Isoform Short of Sodium/potassium- | 4 | Integral membrane protein |
| | | transporting ATPase alpha-1 chain precursor | | |
| 159 | IPI00100081 | Solute carrier family 1 (glutamate transporter), | 7 | Integra membrane protein |
| | | member 7 | | |
| 160 | IPI00301180 | Solute carrier family 12 member 5 | 12 | Integral membrane protein |
| 161 | IPI00299186 | solute carrier family 19 member 1 isoform b | 9 | Integral membrane protein |
| 162 | IPI00003909 | Solute carrier family 2, facilitated glucose transporter, | 10 | Integral membrane protein |
| | | member 3, or 14 | | |
| 163 | IPI00027281 | Solute carrier family 2, facilitated glucose transporter, | 12 | Integral membrane protein |
| | | member 4 | | |
| 164 | IPI00220194 | Solute carrier family 2, facilitated glucose transporter, | 12 | intearal membrane protein |
| | | member 1 | | |
| | GiP11166 | Glucose transporter type I | 12 | Integral membrane protein |
| | Gi3387905 | Glucose transporter glycoprotein | 8 | Integral membrane protein |
| 165 | IPI00008616 | Splice Isoform 1 of Q9Y666 Solute carrier family 12 | 11 | Integral membrane protein |
| | | member 7 | | |
| 166 | IPI00021089 | Solute carrier family 27 (fatty acid transporter), | 2 | Intearal membrane protein |
| | | member 4 | | |
| 167 | IPI00412547 | Solute carrier family 29 (nucleoside transporters) | 11 | Integral membrane protein |
| | | member 1 | | |
| 168 | IPI00005547 | Solute carrier family 40, member 1 | 10 | Integral membrane protein |
| 169 | IPI00301100 | Solute carrier family 43, member 3 | 11 | Integral membrane protein |
| 170 | IPI00377081 | Stomatin | 1 | Cytoskeleton |
| | IPI00219682 | Stomatin isoform a | 1 | Integral membrane protein |
| | | Erythrocyte band 7 integral membrane protein | | Integral membrane protein |
| 171 | P27105 | (stomatin) (protein 7.2B) | | |
| 172 | IPI00011578 | Stromal cell-derived receptor-1 alpha | 1 | Extracellular; Integral membrane protein |
| | IPI00018311 | Stromal cell-derived receptor-1 beta | 1 | Extracellular: Integral membrane protein |
| 173 | IPI00399142 | Surfeit 4 | 2 | Integral membrane protein. ER membrane |
| 174 | IPI00029730 | Syntaxin 4 | 1 | Type IV membrane protein |
| 175 | IPI00289876 | Syntaxin 7 | 1 | Type IV membrane protein |
| | IPI00552913 | Splice Isoform 2 of Syntaxin-7 | 0 | Membrane; Cytoplasmic |
| 176 | IPI00253036 | Splice Isoform I of P14209 T-cell surface | 2 | Integral membrane protein |
| | | glycoprotein E2 precursor | | |
| 177 | P36897 | TGF-beta receptor type I precursor | 2 | Integral membrane protein |
| 178 | IPI00395887 | Thioredoxin domain containing protein 1 precursor | 3 | ER lumen |
| | | Protein disulfide-isomerase A6 precursor | | |
| 179 | IPI00100247 | Thioredoxin-like protein KIAA1162 precursor | 1 | Type I membrane protein |
| 180 | IPI00296099 | Thrombospondin 1 precursor glycoprotein IV, also in | 0 | Extracellular region |
| | | mature RBCs | | |
| 181 | IPI00028642 | Splice Isoform 1 Of Thyrotropin receptor precursor | 0 | Integral membrane protein |
| 182 | IPI00007052 | TPR repeat containing protein | 1 | Integral membrane protein |
| 183 | IPI00394781 | Transmembrane protein 24 | 1 | Integral membrane protein |
| 184 | IPI00028055 | Transmembrane protein Tmp21 precursor | 2 | Type I membrane protein |
| 185 | IPI00332278 | Splice Isoform 2 of Transmembrane protein 55B | 2 | Integral, membrane protein |
| 186 | IPI00220272 | Triadin | 1 | Integral membrane protein |
| 187 | IPI00024466 | UDP-glucose:glycoprotein glucosyltransferase | 1 | Integral membrane protein |
| | | 1 precursor | | |
| 188 | IPI00020515 | Uncharacterised hematopoietic stem/progenitor cells | 1 | Type II membrane protein |
| | | protein MDS032 | | |
| 189 | IPI00007061 | UPF0198 protein CGI-141 | 3 | Integral membrane protein |
| 190 | IPI00298337 | Urea transporter, erythrocyte | 8 | Integral membrane protein |
| 191 | IPI00018855 | Vacuolar ATP synthase 16 kDa proteolipid subunit | 4 | Integral membrane protein |
| 192 | IPI00552514 | Splice Isoform 1 of Vacuolar proton translocating | 7 | Integral membrane protein |
| | | ATPase 116 kDa subunit a isoform 1 | | |
| 193 | IPI00006865 | Vesicle trafficking protein SEC22b | 1 | Type IV membrane protein |
| 194 | IPI00170692 | Vesicle-associated membrane protein-associated | 1 | Type IV membrane protein |
| | | protein A isoform 2 | | |
| 195 | IPI00374657 | Vesicle-associated membrane protein-associated | 1 | Membrane: Cytoskeleton |
| | | protein A isoform 1 | | |
| 196 | IPI00006211 | Splice Isoform 1 of O95292 Vesicle-associated | 1 | Type IV membrane protein |
| | | membrane protein associated protein B/C | | |
| 197 | Gi7657675 | Vesicle-associated membrane protein 2 | 1 | Type IV membrane protein |
| 198 | IPI00009950 | Vesicular integral-membrane protein VIP36 precursor | 1 | Type I membrane protein |
| 199 | P56703 | WNT-3 proto-oncogene protein [precursor] | 1 | Integral membrane protein |
| 200 | IPI00216069 | Splice Isoform 2 of Zinc finger DHHC domain | 4 | Integral membrane protein |
| | | containing protein 3 | | |
| 201 | IPI00002483 | Zinc transporter 1 | 6 | Integral membrane protein |
| 202 | Gi5902116 | Zona pellucida binding protein | 1 | Integral membrane protein |

Membrane Proteins Common to Both AARBCs and FPNRBCs

31 proteins were common to both cell types. These included: structural proteins such as the erythrocyte band 7 integral-membrane protein, ankyrin, spectrin, dematin, Protein 4.1; proteins with transport function such as band 3, aquaporin, calcium-transporting ATPase, sodium/potassium-transporting ATPase, solute carrier family 2, facilitated glucose transporter, member 1; and plasma membrane binding proteins like Kell blood group glycoprotein (CD238).

Plasma Membrane Proteins Unique to FPNRBCs

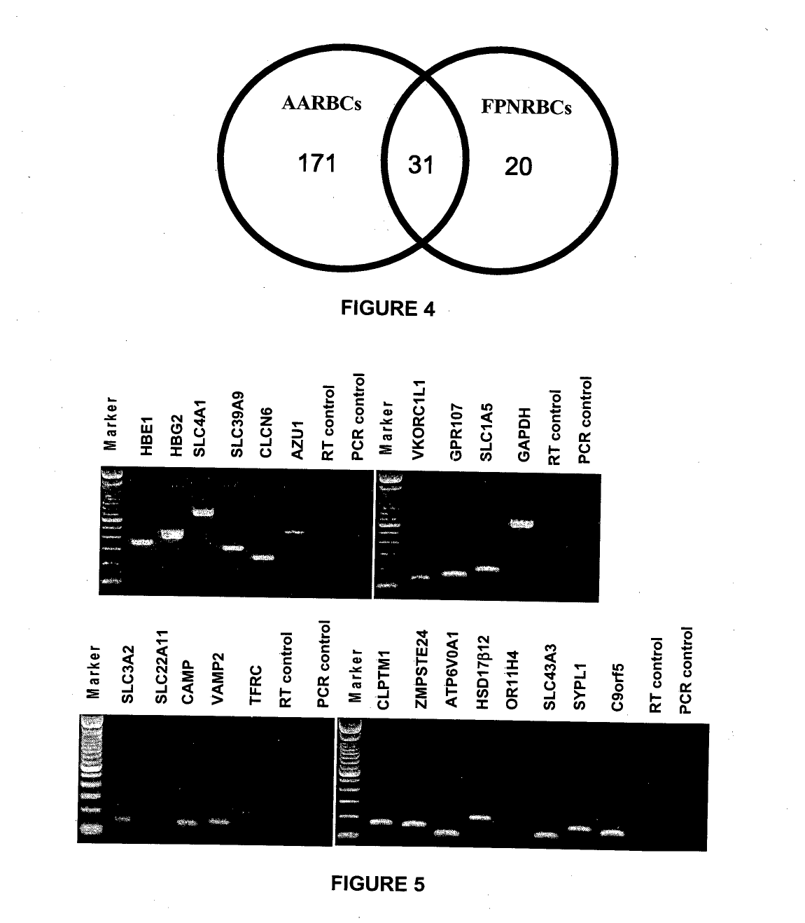

A comparison of membrane proteins with potential surface domains (as annotated) indicated that only 31 proteins were common membrane proteins to AARBCs and FPNRBCs. It was further revealed that 20 proteins were unique to FPNRBCs, and 171 unique to AARBCs, respectively (FIG. 4). Among membrane proteins unique to FPNRBCs, 9 proteins were annotated as being present only on plasma membranes, and 3 others were noted to be present on plasma membranes as well as on ER/Golgi/vesicle membranes (Table 9); but, for 8 other membrane proteins found unique to FPNRBCs, the exact sub-cellular localization was not available (Table 10).

TABLE 9
Unique membrane of proteins FPNRBCs with transmembrane domain

| No | Protein description | IPI Accession # | TMD | Sub-cellular location | Molecular function |
|---|---|---|---|---|---|
| 1 | Neutral amino acid transporter B (SLC1A5) | IPI00019472 | 9 | Plasma membrane | Transporter-Amino acid |
| 2 | Solute carrier family 3 (activators of dibasic and | IPI00554481 | 1 | Plasma membrane | Transporter-Amino acid |
| | neutral amino acid transport), member 2, isoform A | | | | |
| | (SLC3A2) | | | | |
| 3 | Splice Isoform A of Chloride channel protein 6 | IPI00180121 | 3 | Plasma membrane | Transporter-Chloride ion |
| 4 | Transferrin receptor protein 1 | IPI00022462 | 1 | Plasma membrane | Binding and transport-Iron |
| 5 | Splice Isoform 3 of Protein GPR107 precursor | IPI00184474 | 7 | Plasma membrane | Binding receptor-Hormone |
| | | | | | and neurotransmitter |
| 6 | Olfactory receptor 11H4 | IPI00168981 | 7 | Plasma membrane | Binding receptor-Odor |
| 7 | Splice Isoform 1 of Protein C9orf5 | IPI00607576 | 14 | Plasma membrane | Signaling pathways |
| 8 | Cleft lip and palate transmembrane protein 1 | IPI00396411 | 5 | Plasma membrane | Unknown |
| 9 | BCG induced integral membrane protein BIGM103 | IPI00034208 | 7 | Plasma membrane | Antimicrobial |
| 10 | Antibacterial protein FALL-39 precursor | IPI00292532 | 1 | Plasma membrane/ | Antibacterial |
| | | | | Extracellular | |
| 11 | CAAX prenyl protease 1 homolog | IPI0002718O | 7 | Plasma/ER/Golgi | Catalytic |
| | | | | membrane | |
| 12 | Splice Isoform 2 of Synaptophysin-like protein | IPI00335277 | 3 | Plasma/Vesicle | Vesicle recycling |
| | | | | membrane | |

TABLE 10
Unique membrane proteins of FPNRBCs with transmembrane domain but location unknown

| No | Protein description | IPI Accession # | TMD | Sub-cellular location | Molecular function |
|---|---|---|---|---|---|
| 1 | Vitamin K epoxide reductase complex subunit 1- | IPI00166079 | 2 | Unclassified Membrane | Catalytic |
| | like protein 1 | | | (potential) | |
| 2 | Splice Isoform 1 of Protein C20orf22 | IPI00394779 | 1 | Unclassified Membrane | Catalytic |
| | | | | (by similarity) | |
| 3 | Hypothetical protein DKFZp564K247 (Hypoxia | IPI00295621 | 2 | Unclassified Membrane | Unclassified |
| | induced gene 1 protein) | | | (potential) | |
| 4 | Hypothetical protein DKFZp586C1924 | IPI00031064 | 2 | Unclassified Membrane | Unclassified |
| | | | | (potential) | |
| 5 | ALEX3 protein variant | IPI00604615 | 1 | Unclassified Single pass | unclassified |
| | | | | membrane (potential) | |
| 6 | Hypothetical protein MGC14288 | IPI00176708 | 1 | Unclassified Membrane | Unclassified |
| | | | | (potential) | |
| 7 | 8 kDa protein | IPI00639803 | 2 | Unclassified | Unclassified |
| 8 | 25 kDa protein | IPI00646289 | 1 | Unclassified | Unclassified |

Membrane proteins unique to FPNRBCs fall mainly under broad functional groups such as (a) transporter proteins: neutral amino acid transporter B, solute carrier family 3 (activators of dibasic and neutral amino acid transport), splice isoform A of chloride channel protein 6 (chloride ion transport); (b) binding proteins: transferrin receptor protein, splice isoform 3 of Protein GPR107 precursor, olfactory receptor 11H4; and (c) catalytic proteins: CAAX prenyl protease 1 homolog, Vitamin K epoxide reductase complex subunit 1-like protein 1 (VKORC1 L1), Splice Isoform 1 of Protein C20orf22 (ABHD12).

Reverse Transcriptase PCR (RT-PCR) to Confirm Expression of Unique Membrane Proteins within FPNRBCs

FPNRBCs from trophoblastic villi were obtained and all were used to perform the mass spectrometry experiments. To determine if the proteins identified as unique to FPNRBCs were indeed expressed within FPNRBCs, extracted total RNA from FPNRBCs was used to perform an RT-PCR.

mRNA expression of unique proteins of FPNRBCs using total RNA extracted from FPNRBCs and by RT-PCR using primers specific for genes tested (Table 1). The mRNA expression of 23 proteins including 13 proteins unique to FPNRBCs was evaluated (FIG. 5). In FIG. 5, the RT control sample contains no RT enzyme. In the PCR control sample, water was added in place of template. The top panel of FIG. 5 showed the expression of haemoglobin epsilon chain (HBE1), haemoglobin gamma-2 chain (HBG2), solute carrier family 4 member 1 (SLC4A1); solute carrier family 39 member 8 (SLC39A8); chloride channel protein 6 (CLCN6); Azurocidin precursor (AZU1); vitamin K epoxide reductase complex subunit 1-like protein 1 (VKORC1L1); protein GPR107 precursor (GPR107); neutral amino acid transporter B (SLC1A5); Glyceraldehyde-3-phosphate dehydrogenase (GAPDH). The bottom panel of FIG. 5 showed the expression of solute carrier family 3 member 2 (SLC3A2), isoform A; solute carrier family 22 member 11 (SLC22A11), isoform 2; antibacterial protein FALL-39 precursor (CAMP); vesicle-associated membrane protein 2 (VAMP2); transferrin receptor protein 1 (TFRC); cleft lip and palate transmembrane protein 1 (CLPTM1); CAAX prenyl protease 1 homolog; ATP6VOA1 (ZMPSTE24), vacuolar proton translocating ATPase 116 kDa subunit a isoform 1 (ATP6V0A1); steroid dehydrogenase homolog (HSD17β12); solute carrier family 43 member 3 (SLC43A3); synaptophysin-like protein (SYPL1); and protein C9orf5 (C9orf5).

mRNA expression of all the unique proteins on FPNRBCs tested, except olfactory receptor 11H4 (OR11H4), was detected. The absence of amplification of olfactory receptor could probably be due to the low levels of mRNA accumulated as suggested by Feingold and his colleagues.

Immunocytochemical Localization of Unique FPNRBC Proteins

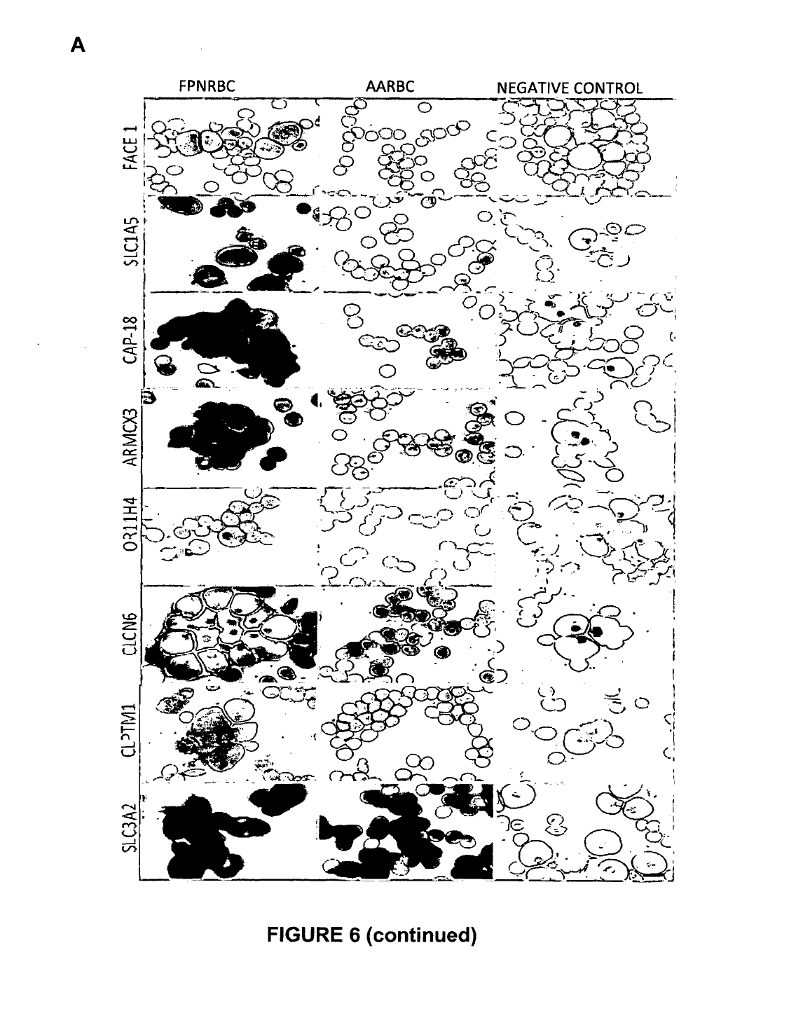

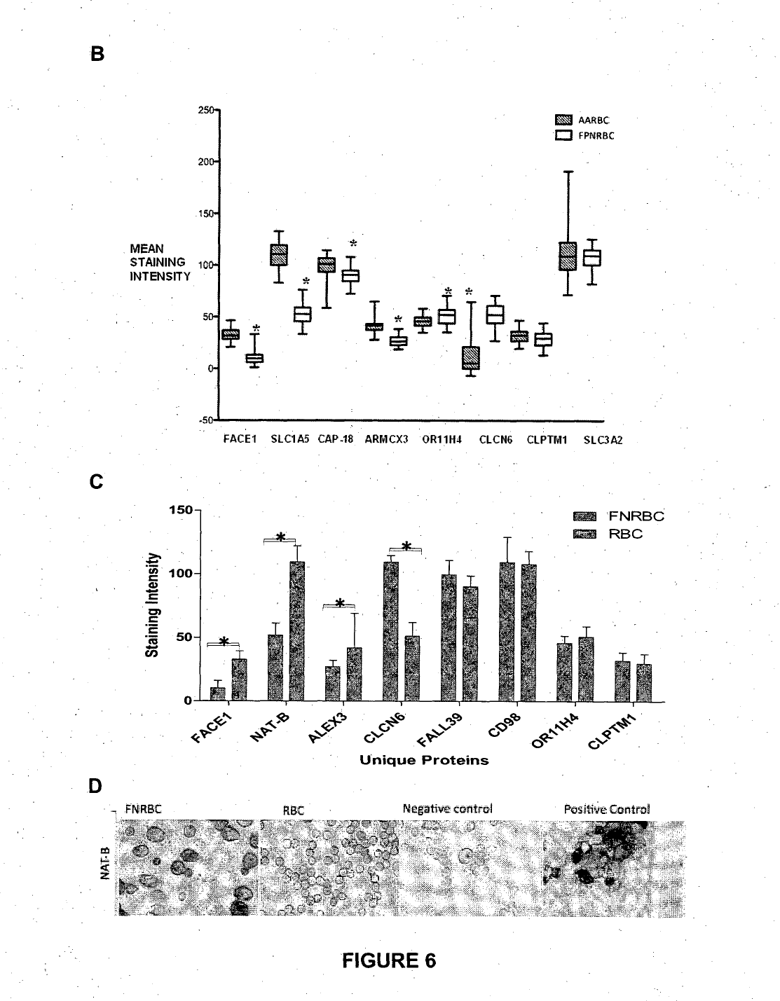

In situ localization of the putative unique FPNRBC proteins was thought to be more informative than western blotting because the location of plasma membrane, cytoplasmic and nuclear proteins could be readily visualized. These were compared to AARBCs. Intensities of immunostaining for the five antibodies tested, FACE-1, SLC1A5, CAP-18, ARMCX3 and OR11H4 were significantly higher (≦0.001) on FPNRBCs than on AARBCs; in contrast, anti-CLCN6 antibody stained AARBCs much more intensely than FPNRBCs (<0.001). There was no significant difference in the staining between FPNRBCs and AARBCs for CLPTM1 and SLC3A2 (FIGS. 6A-B). The Bar represents 10 μm. Bright field images were captured using 20×/0.7 UPlan APO objective lens of BX61 Olympus microscope with Evolution™ MP colour Media Cybernetics CCD camera linked to Image-Pro Discovery software. In FIG. 6B, Mean pixel intensities calculated from the luminosity histogram function on Adobe photoshop CS4 software (Adobe Systems, Mountain View, Calif.) were compared for statistical significance. Mean staining intensity values and intensity of immunoreaction are inversely related.

Intensities of immunostaining of four out of eight antibodies tested were significantly higher (p<0.05; FIGS. 6C and D). Antibodies which are significantly more intense are antibodies towards markers FACE-1, SLC1A5 (NAT-B), ALEX3 (ARMCX3) and CLCN6. In FIG. 6C, mean pixel intensities calculated from the luminosity histogram function on Adobe photoshop CS4 software (Adobe Systems, Mountain View, Calif.) were compared for statistical significance. Mean staining intensity values and intensity of immunoreaction are inversely related. Test used was Mann-Whitney Test, p<0.05 is considered significant.

In FIG. 6D, FPNRBCs extracted from placental villi are relatively larger and identified by the presence of nuclei stained red by nuclear fast stain. FPNRBCs and AARBCs are shown in first and second panels respectively; negative control was carried out by omitting the primary antibody and positive control were run in all experiments.

FPNRBCs Recovery with Anti-NAT-B Antibody

To test the possibility of sorting FPNRBCs using any of the markers found in the present disclosure, adult blood sample was spiked with FPNRBCs. Spike recovery of FPNRBCs using NAT-B (SLC1A5) marker was about 62.5% (FIG. 7B). Sort results are further validated immunohistochemically (FIG. 7A). Immunohistochemical study showed successful recovery of FPNRBCs using NAT-B marker.

Identification of 133 membrane proteins from various sub-cellular locations with different functions would help to explore the importance of FPNRBC in medicine. 132 non-membrane proteins including a few known cytoplasmic proteins (for example, haemoglobin chains ε,γ,δ) are also provided.

Proteomic analyses of FPNRBCs had not been attempted previously owing to the difficulty to obtain sufficient number of cells. Access to placental villi from patients undergoing termination of pregnancy enabled to pool cells for 2D-LCMS/MS analysis. In addition, the extraction of membrane proteins is yet another challenge in proteomics; recovery of more membrane proteins (48.7% of total) from a limited sample (5×10⁷ cells) than those from AARBCs using similar protocol is encouraging, which also explains the structural complexity of these nucleated cells.

Sub-cellular localization and molecular functions annotated for most of the proteins of FPNRBCs are novel for this cell type, which may be useful for protein/developmental/structural biologists, pathologists, haematologists and others. Identified FPNRBC membrane proteins show diverse physiological functions varying from transport, catalytic, binding to structural, while about 32% were transport and/or catalytic. Among the membrane proteins, most were identified from mitochondria (48 proteins) and plasma membrane (37 proteins).

Unique membrane proteins of FPNRBCs were identified to be potential candidates as surface antigens for future separation of this cell type by antibody based techniques. A list of human AARBC membrane proteins prepared based on publications was used for comparison of membrane proteins of FPNRBCs with that of AARBCs: 12 membrane proteins annotated to be in plasma membranes and eight without known sub-cellular locations were found to be unique to FPNRBCs. Proteins with transmembrane domains without known sub-cellular location and molecular function may contain novel antigens of biological significance. This comparison also revealed that 171 proteins are unique to AARBCs which are not found in the data set of FPNRBCs.

A few proteins were found to be common in both the cell types, which included major structural and transport proteins of plasma membrane such as band 3, erythrocyte band 7, facilitated glucose transporter (SLCA2A1), Kell blood group glycoprotein (CD238), aquaporin, ATP-binding cassette half-transporter 1 and glycophorin C, suggesting similar functions for these proteins in FPNRBCs as of their adult counterpart.

In the present disclosure, plasma membrane proteins which are developmental-stage specific to immature red cells but not to AARBCs, such as transferrin receptor and ferritin heavy chain were identified unique to FPNRBCs; similarly, absence of leukocyte specific antigen in the data set also confirms the purity of the samples used.

Indirect validation of unique proteins of FPNRBCs by mRNA expression analysis using RT-PCR revealed the presence of all candidates tested except the olfactory receptor (OR11H4); and the reason for the failure of this protein may probably be due to the low level of the template present in the sample. RT-PCR results for unique proteins confirm their identifications by mass spectrometry. Such validation is not possible for AARBCs as they are mature cells without nuclei or RNA.

Proteomic identification followed by confirmation of their expression in tissues and cells by immunological techniques has been an useful tool in areas such as biomarker discovery, drug discovery and disease biology for example, tumour heterogeneity studies in bladder cancer. Stronger expression levels of unique proteins of FPNRBCs as identified by immunostaining for four of eight antibodies (FACE-1, SLC1A5, CAP-18 and OR11H4) on these cells compared to AARBCs, do support their mass spectrometric identifications. However, expression of chloride channel protein (CLCN6) was found to be opposite (stronger in AARBCs) and two other proteins (SLC3A2 and CLPTM1) did not reveal any difference in their immunostaining in the present study, and such observations may probably be due to the specificity and reactivity of the antibodies used or due to the expression levels and the isoforms of proteins identified. As mentioned earlier, FACE-1 and CAP-18 are also annotated to be present in other locations in addition to their presence in the plasma membrane.

Potential surface antigens for separation of FPNRBCs from maternal blood for non-invasive prenatal diagnosis were identified: these cells in maternal blood, can be separated easily from WBCs using leukocyte specific anti-CD 45 antibody, whereas, it is still challenging to select FPNRBCs from overwhelming AARBCs due to the absence of specific surface antigen present only in any one of these cell types. Identification of unique membrane proteins with transmembrane domains such as FACE-1, SLC1A5, CAP-18 and OR11H4 by mass spectrometry and their intense expressions in FNRBCs, as shown by immunocytochemistry have been done. These potential candidates may be used for separation of this cell type from AARBCs by positive selection by means of immuno-cell sorting techniques such as magnetic activated cell sorting (MACS) or fluorescence activated cell sorting (FACS). Similarly, the absence of immunoreaction of the chloride channel protein in FPNRBCs may also be useful for depletion from AARBCs by such strategies.

Biological Significance of the Unique Plasma Membrane Proteins of FPNRBCs

FIG. 8 shows the locations, and physiological roles (including those related to human foetal development), and diseases related to their mutations of the unique plasma membrane proteins of FPNRBCs.

Briefly, 20 unique membrane proteins could be categorized under seven functional sub-groups: Transportes/Channel molecules: two amino acid transporting Solute Carrier (SLC) proteins, neutral amino acid transporter B0 (NAT-B; SLC1A5, ATB (0), ASCT2), SLC3A2; and an anion transporter, splice isoform A of chloride channel protein 6. Binding proteins: Transferrin receptor protein 1, Splice isoform 3 of protein GPR107 precursor and olfactory receptor 11H4. Catalytic: CAAX prenyl endopeptidase also known as farnesylated protein-converting enzyme (FACE), Vitamin K epoxide reductase complex subunit 1 like protein (VKORC1L1), Splice isoform 1 of protein C20orf22 (ABHD12); Signaling pathway: Splice isoform 1 of Protein C9ORF5; vesicle recycling: Pantopysin; Anti-microbial proteins: BCG induced integral membrane protein BIGM 103 (BCG induced gene in monocyte, clone 103), FALL39; Proteins with no known function: Cleft lip and palate transmembrane protein 1.

Proteins of unknown location and function—reports on protein expression or functional identity of five of the identified proteins of FPNRBCs (with at least one transmembrane domain) are not available in any other cell/tissue; they are, Hypothetical protein DKFZp586C1924, Splice isoform 1 of protein C20orf22 (ABHD12), Hypothetical protein MGC14288, 8 KDa protein and 25 KDa protein. Protein databases searches (UniProtKB/Swiss-Prot) did not reveal much information for these proteins.

These studies on human foetal primitive erythroblasts enables the understanding of the biology of these cells, including haemoglobin switching and regulation of their expression, and, to some extent, on the enrichment of these ideal cells from maternal blood for non-invasive prenatal diagnosis. The proteomic information on the membrane proteins of these cells would help to understand the biology and develop technology for enrichment of these cells from maternal blood for non-invasive prenatal diagnosis.

### Example 2

### Enrichment of FPNRBCs from Post-Termination of Pregnancy (TOP) Maternal Blood Using Anti-ASCT2 Antibody Following Three-Step Enrichment Protocol

10 mls of post-TOP maternal blood was collected from two patients. Blood samples were processed using three-step enrichment protocol of our laboratory. Briefly, diluted blood sample was layered over Percoll 1118 density medium and centrifuged. The interface was collected and white blood cells were depleted by magnetic activated cell sorting (MACS) using anti-CD45 magnetic beads. Cells from negative fraction were incubated with anti-ASCT2 antibody for 30 minutes and washed and again incubated with anti-rabbit IgG-magnetic beads for indirect MACS (positive) selection of FPNRBCs. 20 FPNRBCs could be recovered from each sample (Table 11).

TABLE 11
Enrichment of FPNRBCs from post-TOP maternal blood
using anti-ASCT2 antibody

| Post-TOP Maternal | Gestational age of | Volume | |
|---|---|---|---|
| Blood | foetus | (ml) | FPNRBCs recovered |
| MB1 | 8 + 4 weeks | 10 | 20 |
| MB2 | 8 + 5 weeks | 10 | 20 |

Recovery of Fetal Nucleated Erythroblasts from Model Mixture Experiments Using Antibodies Against ABHD12, GPR107, ORH114 and ALEX3

Fetal nucleated red blood cells were extracted from placental villi and stored in IMDM medium overnight. FPNRBCs and AARBCs in the sample were counted using haemocytometer. Fresh AARBCs were obtained by Ficoll-Plague centrifugation of diluted whole blood at 3,000 rpm for 20 minutes. The pelleted RBCs were collected and washed with 1×PBS and also stored in IMDM medium. AARBCs were spiked into the FPNRBCs-containing tubes such that the concentration of FPNRBCs was maintained at 1-9%. Either 0.5×10⁵ or 1×10⁵ FPNRBCs (depending on the availability of FPNRBCs extracted) were used in the mixtures. Each experiment was carried out in duplicates or triplicates depending on the availability of FPNRBCs extracted.

The cell mixture was pelleted by centrifuging at 2,200 rpm for 5 minutes. Supernatant was removed and appropriate volume of MACS buffer added. The concentration of antibodies for incubation with cell mixture was 1:50 for GPR107; OR11H4 and ABHD12, and 1:100 for ALEX3. After incubation at 4° C. for 30 minutes, cells were washed once at 2,200 rpm for 5 minutes and the buffer supernatant was discarded. 60 μl of MACS buffer and 40 μl of anti-rabbit IgG or anti-mouse IgG beads (Miltenyi) as appropriate were added and incubated at 4° C. for 30 minutes. After washing, the cells were separated using Miltenyi MS columns. The recovery of FPNRBCs from model mixture using anto-GPR107 appeared to be higher (29.4%) than that of OR11H4 and ABHD12, or ALEX3.

TABLE 12
Summary of separation of FPNRBCs from model mixtures
containing adult anudeated RBCs using antibodies against
4 unique surface markers of FNRBCs

| | Positive fraction | | | |
|---|---|---|---|---|
| | FPNRBC | | Negative fraction | |
| Antibody | recovery | AARBC | FPNRBC | AARBC |
| against | (%) | contamination (%) | Lost (%) | depletion (%) |
| ABHD12 | 16.3 | 1.8 | 11.3 | 99.0 |
| GPR107 | 29.4 | 6.0 | 57.2 | 100.0 |
| OR11H4 | 12.6 | 0.9 | 56.6 | 71.3 |
| ALEX3 | 14.0 | 0.2 | 25.0 | 89.7 |

### REFERENCES

1. http://udgenome.ags.udel.edu/gofigure/index.html
2. Lehr H A, Mankoff D A, Corwin D, Santeusanio G, Gown A M. Application of photoshop-based image analysis to quantification of hormone receptor expression in breast cancer. J Histochem Cytochem. 1997; 45:1559-65.
3. Blonder J, Goshe M B, Moore R J, Pasa-Tolic L, Masselon C D, Lipton M S, et al. Enrichment of integral membrane proteins for proteomic analysis using liquid chromatography-tandem mass spectrometry. J Proteome Res. 2002; 1:351-60.
4. Zhang H, Lin Q, Ponnusamy 5, Kothandaraman N, Lim T K, Zhao C, et al. Differential recovery of membrane proteins after extraction by aqueous methanol and trifluoroethanol. Proteomics. 2007; 7:1654-63.

5. Choolani M et al. Simultaneous fetal cell identification and diagnosis by epsilon-globin chain immunophenotyping and chromosomal fluorescence in situ hybridization. Blood 2001, 98:554-557.

1. A method for identifying presence of at least one foetal erythroblast in a sample comprising cells from a subject, comprising:
detecting expression of at least one foetal erythroblast specific marker selected from the group consisting of neutral amino acid transporter B (SLC1A5), solute carrier family 3 (activators of dibasic and neutral amino acid transport) member 2 isoform A (SLC3A2), splice isoform A of chloride channel protein 6, transferrin receptor protein 1, splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, antibacterial protein FALL-39 precursor, CAAX prenyl protease 1 homolog, splice isoform 2 of synaptophysin-like protein, vitamin K epoxide reductase complex subunit 1-like protein 1, splice isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, hypothetical protein MGC14288 (IPI Accession No. IPI00176708), protein with IPI Accession No. IPI00639803, and protein with IPI Accession No. IPI00646289;
wherein detection of the expression of the marker indicates the presence of the foetal erythroblast.

2. The method according to claim 1, wherein the detecting comprises detecting the expression of at least one foetal erythroblast specific marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, antibacterial protein FALL-39 precursor, CAAX prenyl protease 1 homolog, splice isoform 2 of synaptophysin-like protein, and splice isoform 1 of Protein C20orf22 (ABHD12).

3. The method according to claim 1, wherein the detecting comprises detecting the expression of at least one foetal erythroblast specific marker selected from the group consisting of splice isoform 1 of Protein C20orf22 (ABHD12), Splice Isoform 3 of Protein GPR107 precursor, Olfactory receptor 11H4, and ALEX3 protein variant.

4. The method according to claim 1, wherein the foetal erythroblast is of mammalian origin.

5. The method according to claim 4, wherein the foetal erythroblast is of human origin.

6. The method according to claim 1, wherein the marker is detected by an antibody or antigen binding fragment thereof.

7. A method of isolating at least one foetal erythroblast from a sample, the method comprising,
(a) contacting the sample with at least one antibody or antigen binding fragment thereof that is capable of binding to at least one marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289; and
(b) isolating the foetal erythroblast that binds to the antibody or antigen binding fragment thereof from the sample.

8. A method according to claim 7, wherein isolating comprises isolating the foetal erythroblast using a means capable of isolating the foetal erythroblast individually.

9. The method according to claim 8, wherein the means is at least one micromanipulator.

10. The method according to claim 7, wherein the antibody is a polyclonal antibody, a monoclonal antibody, a chimeric antibody, a humanized antibody or a combination thereof.

11. The method according to claim 7, wherein the foetal erythroblast that binds to the antibody is isolated from the sample using immunomagnetic separation, flow cytometry or a combination thereof.

12. An isolated foetal erythroblast obtained according to the method of claim 7.

13. A method of diagnosing at least one prenatal disorder in an individual subject, the method comprising:
(a) identifying at least one foetal erythroblast in a sample from the subject according to claim 1;
(b) isolating the foetal erythroblast; and
(c) determining at least one genetic marker associated with the prenatal disorder in the foetal erythroblast.

14. The method according to claim 13 wherein the prenatal disorder is selected from the group consisting of Down Syndrome, Edwards Syndrome, Patau Syndrome, a neural tube defect, spina bifida, cleft palate, Tay Sachs disease, sickle-cell anemia, thalassemia, cystic fibrosis, fragile X syndrome, spinal muscular atrophy, myotonic dystrophy, Huntington's disease, Charcot-Marie-Tooth disease, haemophilia, Duchenne Muscular Dystrophy, mitochondrial disorder, hereditary multiple exostoses and osteogenesis imperfecta disorder.

15. The method according to either claim 13, wherein the sample is selected from the group consisting of maternal tissue, maternal blood, cord blood, amniocytes, chorionic villus sample, foetal blood, and foetal tissue.

16. The method according to claim 13, wherein the method is in vitro.

17. A marker for identifying at least one foetal erythroblast selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289.

18. An antibody or antigen binding fragment thereof that is capable of binding at least one marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289, wherein the marker is for identifying at least one foetal erythroblast.

19. A kit for identifying and/or isolating at least one foetal erythroblast in a sample, the kit comprising at least one antibody and/or antigen binding fragment thereof that is capable of binding at least one FPNRBC marker selected from the group consisting of neutral amino acid transporter B (SLC1A5), solute carrier family 3 (activators of dibasic and neutral amino acid transport) member 2 isoform A (SLC3A2), Splice Isoform A of Chloride channel protein 6, Transferrin receptor protein 1, Splice Isoform 3 of Protein GPR107 precursor, Olfactory receptor 11H4, Splice Isoform 1 of Protein C9orf5, Cleft lip and palate transmembrane protein 1, BCG induced integral membrane protein BIGM103, Antibacterial protein FALL-39 precursor, CAAX prenyl protease 1 homolog, Splice Isoform 2 of Synaptophysin-like protein, Vitamin K epoxide reductase complex subunit 1-like protein 1, Splice Isoform 1 of Protein C20orf22 (ABHD12), Hypothetical protein DKFZp564K247 (Hypoxia induced gene 1 protein) (IPI Accession No. IPI00295621), Hypothetical protein DKFZp586C1924 (IPI Accession No. IPI00031064), ALEX3 protein variant, Hypothetical protein MGC14288 (IPI Accession No. IPI00176708), protein with IPI Accession No. IPI00639803 and protein with IPI Accession No. IPI00646289.

20. The method according to either claim 7, or 13, wherein the sample is selected from the group consisting of maternal tissue, maternal blood, cord blood, amniocytes, chorionic villus sample, foetal blood, and foetal tissue.


SEQUENCE LISTING (46 sequences)

SEQ ID NO: 1 - DNA, 20 bp, Artificial Sequence
  SLC1A5 forward primer
tggctgctggagtacatgtg

SEQ ID NO: 2 - DNA, 20 bp, Artificial Sequence
  SLC1A5 reverse primer
cccagtgggggctagaattc

SEQ ID NO: 3 - DNA, 20 bp, Artificial Sequence
  SLC3A2 forward primer
atggacccactacccttctc

SEQ ID NO: 4 - DNA, 20 bp, Artificial Sequence
  SLC3A2 reverse primer
catgcaggggtgacttttat

SEQ ID NO: 5 - DNA, 19 bp, Artificial Sequence
  CLCN6 forward primer
gggaccttgtgctgaggga

SEQ ID NO: 6 - DNA, 18 bp, Artificial Sequence
  CLCN6 reverse primer
agctgcgactgcggcaat

SEQ ID NO: 7 - DNA, 23 bp, Artificial Sequence
  TFRC forward primer
taggcagcagcttttaatacagg

SEQ ID NO: 8 - DNA, 24 bp, Artificial Sequence
  TFRC reverse primer
aaagtaagcgaaccacttacaacc

SEQ ID NO: 9 - DNA, 21 bp, Artificial Sequence
  GPR107 forward primer
tcagaacatggttgttctccc

SEQ ID NO: 10 - DNA, 20 bp, Artificial Sequence
  GPR107 reverse primer
gcttgctcttcctccacatc

SEQ ID NO: 11 - DNA, 21 bp, Artificial Sequence
  OR11H4 forward primer
aacaactgaatgtctctttct

SEQ ID NO: 12 - DNA, 21 bp, Artificial Sequence
  OR11H4 reverse primer
ggagtcgttactgaatatacc

SEQ ID NO: 13 - DNA, 20 bp, Artificial Sequence
  C9orf5 forward primer
tagccctgaccttgcagtct

SEQ ID NO: 14 - DNA, 22 bp, Artificial Sequence
  C9orf5 reverse primer
gcatttggaagtaatgctagcc

SEQ ID NO: 15 - DNA, 18 bp, Artificial Sequence
  CLPTM1 forward primer
aggttcccacagcagcag

SEQ ID NO: 16 - DNA, 18 bp, Artificial Sequence
  CLPTM1 reverse primer
cctctgctggctttggag

SEQ ID NO: 17 - DNA, 22 bp, Artificial Sequence
  SLC39A8 forward primer
gtctgagatgcctggtatatag

SEQ ID NO: 18 - DNA, 23 bp, Artificial Sequence
  SLC39A8 reverse primer
tctttggctccttaaagacttgg

SEQ ID NO: 19 - DNA, 22 bp, Artificial Sequence
  CAMP forward primer
gataacaagagatttgccctgc

SEQ ID NO: 20 - DNA, 20 bp, Artificial Sequence
  CAMP reverse primer
gggtagggcacacactagga

SEQ ID NO: 21 - DNA, 21 bp, Artificial Sequence
  ZAMPSTE24 forward primer
cctaaggctaaagaggagcag

SEQ ID NO: 22 - DNA, 20 bp, Artificial Sequence
  ZMPSTE24 reverse primer
gcgttggcaatgtttaatgt

SEQ ID NO: 23 - DNA, 23 bp, Artificial Sequence
  SYPL1 forward primer
tgcatcataaaggaacctaagtg

SEQ ID NO: 24 - DNA, 25 bp, Artificial Sequence
  SYPL1 reverse primer
tgtaagaataagaaacctgaatccc

SEQ ID NO: 25 - DNA, 20 bp, Artificial Sequence
  VKORC1L1 forward primer
agacacctcaggcagcactt

SEQ ID NO: 26 - DNA, 20 bp, Artificial Sequence
  VKORC1L1 reverse primer
tatttcaccttttctgggcg

SEQ ID NO: 27 - DNA, 19 bp, Artificial Sequence
  VAMP2 forward primer
agtcccttaacctgccacg

SEQ ID NO: 28 - DNA, 20 bp, Artificial Sequence
  VAMP2 reverse primer
ctgggataatatggggggtc

SEQ ID NO: 29 - DNA, 25 bp, Artificial Sequence
  HBE1 forward primer
ttttactgctgaggagaaggctgcc

SEQ ID NO: 30 - DNA, 26 bp, Artificial Sequence
  HBE1 reverse primer
cttgccaaagtgagtagccagaataa

SEQ ID NO: 31 - DNA, 22 bp, Artificial Sequence
  HBG2 forward primer
acgccatgggtcatttcacaga

SEQ ID NO: 32 - DNA, 22 bp, Artificial Sequence
  HBG2 reverse primer
gagctcagtggtatctggagga

SEQ ID NO: 33 - DNA, 20 bp, Artificial Sequence
  SLC4A1 forward primer
acacagctcttcgtggagca

SEQ ID NO: 34 - DNA, 20 bp, Artificial Sequence
  SLC4A1 reverse primer
tccgacactcccatctggtt

SEQ ID NO: 35 - DNA, 24 bp, Artificial Sequence
  GAPDH forward primer
aaggactcatgaccacagtccatg

SEQ ID NO: 36 - DNA, 24 bp, Artificial Sequence
  GAPDH reverse primer
ttgatggtacatgacaaggtgcgg

SEQ ID NO: 37 - DNA, 18 bp, Artificial Sequence
  ATP6V0A1 forward primer
acctgacccgaccttgtg

SEQ ID NO: 38 - DNA, 21 bp, Artificial Sequence
  ATP6V0A1 reverse primer
ctgaactctgcttcaaacccc

SEQ ID NO: 39 - DNA, 20 bp, Artificial Sequence
  SLC34A3 forward primer
atgtcctagaaggttttagg

SEQ ID NO: 40 - DNA, 20 bp, Artificial Sequence
  SLC34A3 reverse primer
caaagatagtctgtcagaaa

SEQ ID NO: 41 - DNA, 24 bp, Artificial Sequence
  HSD16B12 forward primer
tgaaatatgcagcaagaagattgg

SEQ ID NO: 42 - DNA, 23 bp, Artificial Sequence
  HSD17B12 reverse primer
aatgatgctgatagcagatggct

SEQ ID NO: 43 - DNA, 24 bp, Artificial Sequence
  AZU1 forward primer
gtgctgggtgcctatgacctgagg

SEQ ID NO: 44 - DNA, 23 bp, Artificial Sequence
  AZU1 reverse primer
aagagcgccactcgggtgaagaa

SEQ ID NO: 45 - DNA, 19 bp, Artificial Sequence
  SLC22A11 forward primer
ctgggttccaatctcaccc

SEQ ID NO: 46 - DNA, 20 bp, Artificial Sequence
  SLC22A11 reverse primer
tttttctggcagctctctca